(19) United States
(12) Patent Application Publication
(10) Pub. No.: US 2023/0233772 A1
(43) Pub. Date: Jul. 27, 2023

(54) METHODS, SYSTEMS AND DEVICES FOR ADMINISTERING MEDICATION

(71) Applicant: Ethan Pedde, Glasgow, MT (US)

(72) Inventor: Ethan Pedde, Glasgow, MT (US)

(21) Appl. No.: 18/126,347

(22) Filed: Mar. 24, 2023

Related U.S. Application Data
(63) Continuation-in-part of application No. 17/349,902, filed on Jun. 16, 2021.
Application No. 17/349,902 is a division of application No. 15/706,662, filed on Sep. 15, 2017, now Pat. No. 11,083,851.
(60) Provisional application No. 62/396,101, filed on Sep. 17, 2016.

(51) Int. Cl. A61M 5/315; A61M 5/24; A61M 5/31; A61M 5/50; A61M 5/00; A61M 5/32; A61J 1/20; A61M 5/178

(52) U.S. Cl. CPC A61M 5/31545; A61M 5/24; A61M 5/3129; A61M 5/3148; A61M 5/31535; A61M 5/502; A61M 5/002; A61M 5/3213; A61J 1/2055; A61M 5/1782; A61M 5/31536; A61J 1/201; A61J 1/2096; A61M 2210/04; A61M 2005/5033

(57) ABSTRACT

Systems, methods and devices for administering medication are disclosed. One or more embodiments of a medication delivery device may include a syringe, a needle, a guiding element, and a volume limiter for limiting the amount of medication that can be drawn from a vial. At least one method for administering medication using a medication delivery device, may include: inserting a vial into a guiding element, drawing back on a plunger, removing the guiding element and the vial from a needle to expose the needle, inserting the needle into a patient, depressing the plunger to inject the medication into the patient, and withdrawing the medication delivery device from the patient.

### CROSS-REFERENCES TO RELATED APPLICATIONS

This application is a continuation-in-part application of and claims the benefit of and priority to U.S. patent application Ser. No. 17/349,902, which is entitled “METHODS, SYSTEMS AND DEVICES FOR ADMINISTERING MEDICATION,” (herein “Patent App. 902”) and which was filed on Jun. 16, 2021.

Patent App. 902 was published as U.S. Patent Application Publication No. US 2021/0308383 A1 (herein “Pub. 383”) on Oct. 7, 2021, and is entitled “METHODS, SYSTEMS AND DEVICES FOR ADMINISTERING MEDICATION.” Patent App. 902 is a divisional application of and claims the benefit of and priority to U.S. patent application Ser. No. 15/706,662, which is entitled “METHODS, SYSTEMS AND DEVICES FOR ADMINISTERING MEDICATION,” (herein “Patent App. 662”) and which was filed on Sep. 15, 2017.

Patent App. 662 was published as U.S. Patent Application Publication No. US 2018/0078710 A1 (herein “Pub. 710”) on Mar. 22, 2018, and is entitled “METHODS, SYSTEMS AND DEVICES FOR ADMINISTERING MEDICATION.” Patent App. 662 was later issued as U.S. Pat. No. 11,083,851 B2 (herein “Patent 851”) on Aug. 10, 2021, and is entitled “METHODS, SYSTEMS AND DEVICES FOR ADMINISTERING MEDICATION.” Patent App. 662 claims the benefit of and priority to U.S. Provisional Patent Application No. 62/396,101, which is entitled “ENHANCED SYSTEM FOR ADMINISTRATION OF INJECTABLE MEDICATIONS” (hereafter “Provisional App. 101”), and which was filed on Sep. 17, 2016.

The entire contents of Patent App. 902, Pub. 383, Patent App. 662, Pub. 710, Patent 851, and Provisional App. 101 are hereby incorporated herein by reference as if set forth in full and made apart of this application.

### BACKGROUND

### Technical Field

The disclosure relates at least to methods, systems, and devices for administering medication.

### Discussion of Related Field

Many people have medical conditions that require themselves or others to administer medication to them regularly, infrequently or in emergency or other situations.

In general terms, adverse working conditions may make administering injectable medication difficult. Adverse working conditions may include low lighting, bad weather, wearing winter gloves and other clothing or equipment, poor eyesight, unfamiliarity, ambulatory motion, stress, high-volume vaccinations, and other conditions that may make it difficult to handle, read (such as reading the graduation, if any, on a syringe) or otherwise administer medication. There is at least a need for improved medication delivery devices, methods and systems that allow a user to more easily administer medication under adverse working conditions.

Administering too much or too little medication may be harmful to patients. Current approaches to administering medication may require fine measurements and good eyesight to ensure accurate dosage (such as drawing a plunger to a specific graduation). Adverse working conditions, such as those indicated above, may make it difficult to administer medication accurately. As such, there is at least a need for improved medication delivery devices, methods and systems that may ensure that the correct dosage is administered, that prevent overdosing and that reduce the need for a user to exercise fine motor skills and have good eyesight to ensure accurate dosage.

Current approaches to administering medication may require a user to be trained in order to properly use current approaches to administering medication. As such, there is at least a need for improved medication delivery devices, methods and systems that may be used by individuals with limited training (such as diabetics giving themselves insulin injections or anemia patients giving themselves routine B12 injections).

Current approaches to administering medication may allow individuals to use the same needle to access and extract medication from the vial and inject it. However, such approaches may use unshielded needles which run the risk of being damaged (such as being broken or bent), causing damage to others (such as sticking individuals administering the medication) or being contaminated. Damaged and contaminated needles and inadvertent needle-sticks necessitate changing the needle before injecting. Not only does such waste resources but it may cost valuable time, especially in adverse working conditions (such as emergency situations). Furthermore, if one uses rubber-septum vials, under current approaches one may need to use an unshielded needle to access the medication while taking care not to blunt or bend the needle through mishandling. There is at least a need for improved medication delivery devices, methods and systems that allow a user to use a shielded needle when administering medication in order to reduce or eliminate the risk of causing damage to or contamination of the needle and to prevent needle sticks.

Current approaches to administering medication may require the needle to be carefully held in the vial below the medication level. Fine motor skills and other skills may be required to perform such approaches, which requirement may be hampered by adverse working conditions, especially in crisis or emergency situations. There is at least a need for improved medication delivery devices, methods and systems that reduce the level of fine motor skills a user must exercise when securing the needle in the vial below the medication level, including under adverse working conditions.

With regards to drawing a dose with a standard syringe, current approaches may require a user to properly center a needle in three dimensions and push all the way through the septum of the vial while taking care not to blunt the needle on the bottom or sides of the vial. The user may be required to push air into the vial to relieve the vacuum and draw back while watching the graduations. As such, there is at least a need for improved medication delivery devices, methods and systems that center the needle in the vial, set the tip at the proper depth and stop retraction at the appropriate volume to prevent overdosing.

Current approaches to administering medication may use a vial access or guiding device to access a vial and a needle to inject the medication. However, in order to use currently available vial access or guiding devices for non-IV (or non-port) injections, the vial access or guiding devices must be removed from the syringe and a separate needle must then be attached to the syringe before injecting a patient. Having to both remove the vial access or guiding device and then attach the needle takes additional time, requires an additional step and may increase the possibility of error, damage and contamination. As such, there is at least a need for improved medication delivery devices, methods and systems that configure the needle and the vial access or guiding device in such a way that eliminates the need to attach the needle to the syringe after the vial access or guiding device has been used and before injecting the patient.

Current auto-injectors may include instructions to press the auto-injector firmly against the injection site. Such instructions may often be misinterpreted by end-users who overenthusiastically hit or otherwise engage the auto-injector against the site, resulting in additional pain and possible contusions to the patient. As such, there is at least a need for improved medication delivery devices, methods and systems that use a manual injection process wherein end-users may be less likely to make such mistakes.

Current auto-injectors and cartridge-based systems may be expensive and complex mechanisms. Auto-injections or cartridge-based systems may pose a risk of being misused. As such, there is at least a need for improved medication delivery devices, methods and systems that may be simple to use and manufacture, less expensive and safer.

Current prefilled syringes, auto-injectors and cartridge-based systems may exist for one-dose injections and may be limited in flexibility to only what is available from specific manufacturers. As such, there is at least a need for improved medication delivery devices, methods and systems that may use inserts, flexible guiding elements, adaptable components or other configurations to allow use with any vial.

In light of the above, there is a need for improved medication delivery devices, methods and systems that at least provide a syringe, a needle, a vial access or guiding device and a medication volume limiting device that ensure the correct placement of the needle in the vial, secure the operable connection of the needle and vial while medication is being extracted from the vial, protect the needle from being damaged or causing damage, limit the amount of medication that may be administered and ensure that patients are given the correct dosage without having to rely on graduations on the syringe (if any), and that may be used by individuals with limited training or under adverse working conditions, and that otherwise overcome at least some of the disadvantages and needs stated and apparent above.

### SUMMARY

In one aspect of the disclosure, a medication delivery device may include: a syringe, including: a flange; a syringe barrel for housing medication; a plunger slideably disposed in the syringe barrel for drawing medication from a vial into the syringe barrel and for injecting the medication from the syringe barrel into a patient, wherein at least a portion of the plunger may be configured for limiting the distance the plunger can travel within the syringe barrel; and a syringe tip; a needle; a guiding element comprising a guiding element shaft for housing the needle; and a volume limiter for limiting the amount of medication that can be drawn from the vial.

Implementations may include one or more of the following features. The medication delivery device may include a safety sleeve for covering the needle, wherein the safety sleeve may be operably connectable to the syringe and the needle. The guiding element may operably connect to the needle and include a guiding element barrel for securing the vial and for guiding the needle into the vial. The volume limiter may operably connect to the syringe. The volume limiter may include a top surface; a bottom surface; an opening through which the plunger may be inserted; a first bottom ledge and a second bottom ledge arranged to allow the flange to slide in between and be secured by the first bottom ledge and the second bottom ledge; and a first flange housing element for engaging the flange. The plunger may include an engagement section for limiting the distance the plunger can travel within the syringe barrel; and the volume limiter may include a top surface; a bottom surface; and an opening through which the plunger may be inserted, wherein the opening may include a first surface that assumes the contour of the engagement section of the plunger; and a second surface that assumes the contour of a surface of the plunger alternate to the engagement section. The plunger may include an engagement section for limiting the distance the plunger can travel within the syringe barrel, wherein the engagement section may include a second end that engages the volume limiter to limit the amount of medication that can be drawn from the vial. The plunger may be configured to disengage the second end from the volume limiter, thereby allowing plunger to travel without being restricted by the volume limiter. The plunger may include a channel and the volume limiter may include a pin, wherein the pin may interact with the channel to limit the distance the plunger can travel within the syringe barrel and to limit the amount of medication that can be drawn from the vial. The volume limiter may include a retention member for resiliently securing an operable connection between the volume limiter and the syringe and for automatically engaging the volume limiter to limit the amount of medication that can be drawn from the vial. The volume limiter may be permanently connected to the syringe and include a first flange housing element for engaging the flange; and a second flange housing element for engaging the flange. The volume limiter may include a swivelable plate which engages the engagement section of the plunger to limit the amount of medication that can be drawn from the vial. The volume limiter may be configured into the syringe barrel as at least one protrusion which may engage a second end of the plunger to limit the distance the plunger may travel within the syringe barrel and to limit the amount of medication that can be drawn from the vial. The guiding element shaft may be configured as a semi-sharp cannula and include a neck designed to be sharp enough to puncture a rubber septum of the vial but not sharp enough to penetrate skin; and at least one duct wherein medication may flow from the vial to the needle. The medication delivery device may include a venting needle for allowing air into the vial as medication may be withdrawn from the vial in order to prevent the formation of a vacuum that may inhibit the flow of medication into the syringe. The guiding element may include at least one flexible member for engaging and securing the vial when the vial is inserted into the guiding element barrel. The medication delivery device may include a means for guiding the needle into substantially the center of the vial; and a means for automatically engaging the volume limiter for limiting the amount of medication that can be drawn from the vial. The medication delivery device may include a means for engaging vials of more than one size.

In one aspect a method for administering medication using a medication delivery device which may include: a syringe which may include a flange; a syringe barrel for housing medication; a plunger slideably disposed in the syringe barrel for drawing medication from a vial into the syringe barrel and for injecting the medication from the syringe barrel into a patient, wherein at least a portion of the plunger is configured for limiting the distance the plunger can travel within the syringe barrel; and a syringe tip; a needle; a guiding element which may include a guiding element shaft for housing the needle; and a guiding element barrel for securing the vial and for guiding the needle into the vial; a volume limiter for limiting the amount of medication that can be drawn from the vial; wherein the method for administering medication using the medication delivery device may include: inserting the vial into the guiding element barrel such that the needle accesses the vial below the medication level; drawing back on the plunger to fill the syringe with medication until the volume limiter prevents further travel of the plunger; removing the guiding element and the vial from the needle to expose the needle; inserting the needle into the patient; depressing the plunger to inject the medication into the patient; and withdrawing the medication delivery device from the patient.

In another aspect a method for administering medication using a medication delivery device which may include: a syringe which may include a flange; a syringe barrel for housing medication; a plunger slideably disposed in the syringe barrel for drawing medication from a vial into the syringe barrel and for injecting the medication from the syringe barrel into a patient, wherein at least a portion of the plunger is configured for limiting the distance the plunger can travel within the syringe barrel; and a syringe tip; a needle; a guiding element which may include a guiding element shaft for housing the needle; and a guiding element barrel for securing the vial and for guiding the needle into the vial; a volume limiter for limiting the amount of medication that can be drawn from the vial; wherein the method for administering medication using the medication delivery device may include inserting the vial into the guiding element barrel such that the needle accesses the vial below the medication level; depressing the plunger to force air into the vial; allowing the plunger to automatically draw back to fill the syringe with medication until the volume limiter prevents further travel of the plunger; removing the guiding element and the vial from the needle to expose the needle; inserting the needle into the patient; depressing the plunger to inject the medication into the patient; and withdrawing the medication delivery device from the patient.

These general and specific aspects may be implemented by using systems, apparatuses, devices, means, methods and structures and/or any combination thereof.

Certain implementations may provide one or more of the following advantages. Embodiments may not achieve any or all of the listed advantages. Further, this is not an exhaustive list of all possible advantages of the disclosure. One or more embodiments of the disclosure may be configured to be and/or provide users the following.

In one or more embodiments, the disclosure may prevent accidental overdosing. In one or more embodiments, the disclosure provides a volume limiter or a physical barrier to overdosing, thereby providing a safe process that ensures the correct dosage is administered. In one or more embodiments, the disclosure may include a syringe with a pre-attached needle and a fixed volume limiter to prevent accidental overdosing.

In one or more embodiments, the disclosure may provide for a manual delivery system for medications to be administered by persons with limited training.

In one or more embodiments, the disclosure, when compared to conventional ways of administering medication, may do one, some, none or a combination of the following: eliminate steps when administering medication, eliminate fine measurement steps when administering medication in order to ensure accurate dosage, and reduce costs by eliminating the engineering and materials involved in providing accurate doses.

In one or more embodiments, the disclosure may provide for a safe, convenient, simple, and cost effective means for administering medication. In one or more embodiments, the disclosure may be simple and low cost to manufacture and may be sold at an affordable price and inexpensive to replace. In one or more embodiments, the disclosure may be designed to make the process safe for field use.

In one or more embodiments, the expiration date of the disclosure may not be dependent upon the expiration date of the medication that is to be administered to patients. In one or more embodiments, a patient may be able to replace outdated medication without having to replace the disclosure, thereby allowing users to keep quality medication on hand without having to incur the additional expense of replacing the disclosure. In one or more embodiments, the disclosure may be provided or sold independent of the medication being administered to minimize costs, eliminate short expiration dates and allow the disclosure to be used for administering different medications.

In one or more embodiments, the disclosure may be designed to be easy to use and safe to the individual administering the medication. In one or more embodiments, the disclosure may be designed such that a user is not likely to misuse it (such as using it backwards as is the case with EpiPen injectors) or spend unrecoverable dosage.

In one or more embodiments, the disclosure may be light weight. For example, in one or more embodiments, for about the same weight and general size as a twin-pack of EpiPen injectors, a patient may carry enough of the disclosures and a 1 mL vial of epinephrine to provide up to three doses for adults or six doses for pediatric patients.

In one or more embodiments, the disclosure may be produced in an array of sizes (such as designed to carry a variety of dosages). In one or more embodiments, the disclosure may be able to be used for virtually any type of medication. In one or more embodiments, the disclosure may be designed to be used with multi-dose vials.

In one or more embodiments, the disclosure may be designed to replace regular needle/syringe sets used for IM or SC injections.

Other aspects and advantages may be apparent from the following detailed description, the accompanying drawings, and the claims.

### BRIEF DESCRIPTION OF THE DRAWINGS

Various embodiments of the disclosure will now be discussed with reference to the appended drawings. It is appreciated that these drawings depict only typical embodiments of the disclosure and are not to be considered limiting of its scope.

FIG. 1 shows a side view of one embodiment of a medication delivery device;

FIG. 2 shows a side view of one embodiment of a medication delivery device including a safety shield;

FIG. 3 shows an exploded perspective view of one embodiment of various components of a medication delivery device;

FIG. 4 shows a perspective view of one embodiment of a needle operably connected to one embodiment of a safety shield;

FIG. 5A shows a perspective view of one embodiment of a guiding element and one embodiment of a safety shield;

FIG. 5B shows a perspective view of one embodiment of a guiding element;

FIG. 6 shows a bottom view of one embodiment of a guiding element;

FIG. 7 shows a top view of the embodiment of the guiding element illustrated in FIG. 6;

FIG. 8A shows a cross sectional view of one embodiment of a guiding element and a side view of one embodiment of a vial, wherein the vial is approaching the guiding element;

FIG. 8B shows a cross sectional view of the embodiment of the guiding element illustrated in FIG. 8A, albeit the vial has been inserted into the guiding element barrel;

FIG. 9 shows a cross sectional view of one embodiment of aspects of a guiding element shaft, along with a side view of one embodiment of a needle hub;

FIG. 10A shows a top perspective view of one embodiment of a volume limiter;

FIG. 10B shows a bottom perspective view of the embodiment of the volume limiter illustrated in FIG. 10A;

FIG. 10C shows a side view of the embodiment of the volume limiter illustrated in FIG. 10A, as well as an approaching syringe;

FIG. 10D shows a bottom perspective view of the embodiment of the volume limiter illustrated in FIG. 10A, wherein the flange of the syringe has been inserted into the volume limiter;

FIG. 10E shows a back view of the embodiment of the volume limiter illustrated in FIG. 10A;

FIG. 10F shows a front view of the embodiment of the volume limiter illustrated in FIG. 10A;

FIG. 11A shows a side view of one embodiment of a plunger;

FIG. 11B shows a front view of the embodiment of the plunger illustrated in FIG. 11A;

FIG. 12A shows a perspective view of the embodiment of the plunger illustrated in FIG. 11A approaching the embodiment of the volume limiter illustrated in FIG. 10A and a syringe operably connected to the volume limiter;

FIG. 12B shows a perspective view of the embodiment of the plunger illustrated in FIG. 11A inserted through an opening of the embodiment of the volume limiter illustrated in FIG. 10A and a syringe operably connected to the volume limiter;

FIG. 12C shows a cross sectional view along lines 1-1 of the embodiments of the plunger and volume limiter illustrated in FIG. 12B with a syringe operably connected to the volume limiter, albeit the plunger has been depressed and moved to engage the opening of the volume limiter;

FIG. 12D shows cross sectional views along lines 1-1 of the embodiments of the plunger, volume limiter and syringe illustrated in FIG. 12B, albeit that the plunger has been raised;

FIG. 12E shows a top view of the embodiment of the volume limiter illustrated in FIG. 10A and a horizontal cross sectional view at the engagement section of the embodiment of the plunger illustrated in FIG. 11A, which plunger has been inserted through the opening of the volume limiter;

FIG. 12F shows a perspective view of the embodiment of the plunger illustrated in FIG. 11A inserted through an opening of the embodiment of the volume limiter illustrated in FIG. 10A and twisted;

FIG. 12G shows a top view of the embodiment of the volume limiter illustrated in FIG. 10A and a horizontal cross sectional view at the engagement section of the embodiment of the plunger illustrated in FIG. 11A, which plunger has been inserted through the opening of the volume limiter and twisted;

FIG. 12H shows a cross sectional view along lines 1-1 of the embodiments of the plunger, volume limiter and syringe illustrated in FIG. 12B, albeit the plunger has been twisted and raised;

FIG. 13A shows a bottom perspective view of the embodiment of the volume limiter illustrated in FIG. 10D, albeit the volume limiter shown in FIG. 13A includes a retention member;

FIG. 13B shows a top perspective view of the embodiment of the volume limiter illustrated in FIG. 13A;

FIG. 14 shows a cross sectional view of another embodiment of a volume limiter;

FIG. 15 shows a cross sectional view of another embodiment of a volume limiter;

FIG. 16A shows a cross sectional view of one embodiment of a volume limiter configured as a pin and one embodiment of a syringe including a flange;

FIG. 16B shows a top view of the embodiments of the flange and pin illustrated in FIG. 16A;

FIG. 17A shows a cross sectional view of another embodiment of a volume limiter;

FIG. 17B shows a perspective view of the embodiment of the volume limiter illustrated in FIG. 17A;

FIG. 18A shows a top view of one embodiment of a volume limiter including a swivelable plate;

FIG. 18B shows a cross sectional view along lines 2-2 of the embodiment of the volume limiter including a swivelable plate as illustrated in FIG. 18A;

FIG. 19A shows a cross sectional view of another embodiment of a volume limiter;

FIG. 19B shows a top perspective view of the embodiment of the volume limiter illustrated in FIG. 19A;

FIG. 20 shows a cross sectional view of one embodiment of a syringe including one embodiment of a volume limiter built into a syringe barrel;

FIG. 21A shows a cross sectional view of another embodiment of a syringe including one embodiment of a volume limiter built into a syringe barrel;

FIG. 21B shows a horizontal cross sectional view at the syringe barrel's protrusions of the embodiments of the syringe and plunger illustrated in FIG. 21A;

FIG. 21C shows a horizontal cross sectional view at the syringe barrel's protrusions of the embodiments of the syringe and plunger illustrated in FIG. 21A, wherein the plunger has been rotated;

FIG. 22A shows a cross sectional view of one embodiment of a plunger including one embodiment of volume limiter built into the plunger;

FIG. 22B shows a horizontal cross sectional view at the first wall and second wall sections of the embodiment of the syringe illustrated in FIG. 22A;

FIG. 22C shows a horizontal cross sectional view at the first wall and second wall sections of the embodiment of the syringe illustrated in FIG. 22A, wherein the plunger has been rotated;

FIG. 23 shows a cross sectional view of one embodiment of a syringe including one embodiment of a volume limiter built into a syringe barrel;

FIG. 24A shows a cross sectional view of one embodiment of a volume limiter configured as a single collar;

FIG. 24B shows a perspective view of the embodiment of the volume limiter illustrated in FIG. 24A;

FIG. 24C shows a perspective view of the embodiment of the syringe barrel illustrated in FIG. 24A including at least one flexible section and a pocket;

FIG. 25A shows a cross sectional view of one embodiment of a volume limiter configured as an articulable door;

FIG. 25B shows a cross sectional view of the embodiment of the volume limiter illustrated in FIG. 25A, albeit the articulable door has been disengaged from a flange;

FIG. 26A shows a top view of one embodiment of a volume limiter configured as a detachable collar;

FIG. 26B shows a side view of the embodiment of the volume limiter illustrated in FIG. 26A;

FIG. 26C shows a top view of the embodiment of the volume limiter as illustrated in FIG. 26A, albeit a plunger assumes a cruciform configuration;

FIG. 26D shows a top view of one embodiment of a volume limiter similar to the embodiment of the volume limiter illustrated in FIG. 26A, albeit a wing member is smaller, a handle is larger and a first arm member and second arm member assume an alternative design;

FIG. 26E shows a top view of one embodiment of a volume limiter similar to the embodiment of the volume limiter illustrated in FIG. 26A, albeit a wing member is replaced with a protrusion;

FIG. 27A shows a side view of one embodiment of a needle and a cross sectional view of one embodiment of a guiding element which may include a transition section;

FIG. 27B shows a side view of the embodiment of the needle and a cross sectional view of the embodiment of the guiding element illustrated in FIG. 27A, albeit the guiding element includes a venting needle;

FIG. 28A shows a cross sectional view of one embodiment of a guiding element including at least two bow members;

FIG. 28B shows a bottom view of the embodiment of the guiding element illustrated in FIG. 28A;

FIG. 29A shows a cross sectional view of one embodiment of a guiding element including at least three fins;

FIG. 29B shows a bottom view of the embodiment of the guiding element illustrated in FIG. 29A;

FIG. 30A shows a cross sectional view of one embodiment of a guiding element including a guiding element barrel with independently movable sections of its wall;

FIG. 30B shows a bottom view of the embodiment of the guiding element illustrated in FIG. 30A;

FIG. 31A shows a cross sectional view of one embodiment of a guiding element including a guiding element barrel, at least a portion of which may be designed with flexible or semi-flexible material;

FIG. 31B shows a bottom view of the embodiment of the guiding element illustrated in FIG. 31A;

FIG. 32A shows a cross sectional view of one embodiment of a guiding element including at least one flexible member;

FIG. 32B shows a bottom view of the embodiment of the guiding element illustrated in FIG. 32A;

FIG. 33 shows a cross sectional view of one embodiment of a guiding element with at least one insert;

FIG. 34A shows a cross sectional view of one embodiment of a guiding element including a guiding element shaft configured like a semi-sharp cannula that includes a venting channel;

FIG. 34B shows a perspective view of the embodiment of the guiding element illustrated in FIG. 34A;

FIG. 35A shows a cross sectional view of one embodiment of a guiding element with a neck assuming a cone configuration and a duct assuming a “Y” configuration;

FIG. 35B shows a perspective view of the embodiment of the guiding element illustrated in FIG. 35A;

FIG. 36 shows a perspective view of one embodiment of a medication delivery device in a blister package;

FIG. 37 shows a flow diagram that depicts one embodiment of a method for using a medication delivery device;

FIG. 38 shows a flow diagram that depicts one embodiment of another method for using a medication delivery device;

FIG. 39 shows a top perspective view of one embodiment of a guiding element;

FIG. 40 shows a bottom perspective view of the embodiment of the guiding element illustrated in FIG. 39;

FIG. 41 shows a top view of the embodiment of the guiding element illustrated in FIG. 39;

FIG. 42 shows a bottom view of the embodiment of the guiding element illustrated in FIG. 39;

FIG. 43 shows a right side view of the embodiment of the guiding element illustrated in FIG. 39;

FIG. 44 shows a left side view of the embodiment of the guiding element illustrated in FIG. 39;

FIG. 45 shows a front view of the embodiment of the guiding element illustrated in FIG. 39;

FIG. 46 shows a rear view of the embodiment of the guiding element illustrated in FIG. 39;

FIG. 47 shows a perspective view of the embodiment of the guiding element illustrated in FIG. 39 shown in an environment of use;

FIG. 48 shows a top perspective view of one embodiment of a guiding element;

FIG. 49 shows a bottom perspective view of the embodiment of the guiding element illustrated in FIG. 48;

FIG. 50 shows a top view of the embodiment of the guiding element illustrated in FIG. 48;

FIG. 51 shows a bottom view of the embodiment of the guiding element illustrated in FIG. 48;

FIG. 52 shows a right side view of the embodiment of the guiding element illustrated in FIG. 48;

FIG. 53 shows a left side view of the embodiment of the guiding element illustrated in FIG. 48;

FIG. 54 shows a front view of the embodiment of the guiding element illustrated in FIG. 48;

FIG. 55 shows a rear view of the embodiment of the guiding element illustrated in FIG. 48;

FIG. 56 shows a perspective view of the embodiment of the guiding element illustrated in FIG. 48 shown in an environment of use;

FIG. 57 shows a bottom view of the embodiment of the guiding element illustrated in FIG. 42, albeit without the shading;

FIG. 58 shows a bottom view of a portion of the embodiment of the guiding element illustrated in FIG. 42 with a vial inserted into it;

FIG. 59 shows a side view of the embodiment of the guiding element illustrated in FIG. 57;

FIG. 60 shows a side view of the embodiment of the guiding element illustrated in FIG. 58 with the vial inserted into it;

FIG. 61 shows a top perspective view of one embodiment of a guiding element;

FIG. 62 shows a bottom perspective view of the embodiment of the guiding element illustrated in FIG. 61;

FIG. 63 shows a top view of the embodiment of the guiding element illustrated in FIG. 61;

FIG. 64 shows a bottom view of the embodiment of the guiding element illustrated in FIG. 61;

FIG. 65 shows a right side view of the embodiment of the guiding element illustrated in FIG. 61;

FIG. 66 shows a left side view of the embodiment of the guiding element illustrated in FIG. 61;

FIG. 67 shows a front view of the embodiment of the guiding element illustrated in FIG. 61;

FIG. 68 shows a rear view of the embodiment of the guiding element illustrated in FIG. 61;

FIG. 69 shows a perspective view of the embodiment of the guiding element illustrated in FIG. 61 shown in an environment of use;

FIG. 70 shows a top perspective view of one embodiment of a guiding element;

FIG. 71 shows a bottom perspective view of the embodiment of the guiding element illustrated in FIG. 70;

FIG. 72 shows a top view of the embodiment of the guiding element illustrated in FIG. 70;

FIG. 73 shows a bottom view of the embodiment of the guiding element illustrated in FIG. 70;

FIG. 74 shows a right side view of the embodiment of the guiding element illustrated in FIG. 70;

FIG. 75 shows a left side view of the embodiment of the guiding element illustrated in FIG. 70;

FIG. 76 shows a front view of the embodiment of the guiding element illustrated in FIG. 70;

FIG. 77 shows a rear view of the embodiment of the guiding element illustrated in FIG. 70;

FIG. 78 shows a perspective view of the embodiment of the guiding element illustrated in FIG. 70 shown in an environment of use;

FIG. 79 shows a bottom view of the embodiment of the guiding element illustrated in FIG. 64, albeit without the shading;

FIG. 80 shows a cross sectional view along lines 3-3 of the embodiment of the guiding element illustrated in FIG. 79;

FIG. 81 shows a cross sectional view along lines 3-3 of the embodiment of the guiding element illustrated in FIG. 79 with the guiding element engaging a vial;

FIG. 82 shows a cross sectional view along lines 3-3 of the embodiment of the guiding element illustrated in FIG. 79 with the guiding element engaging an alternative vial;

FIG. 83 shows a perspective view of one embodiment of a receiving boot operably connected to one embodiment of a guiding element;

FIG. 84 shows an alternative view of the embodiments of the receiving boot and guiding element illustrated in FIG. 83, albeit the slits assume an alternative configuration;

FIG. 85 shows an alternative perspective view of the embodiments of the receiving boot and guiding element illustrated in FIG. 83;

FIG. 86 shows a needle with one embodiment of a sliding safety shield operably connected to it approaching the receiving boot illustrated in FIG. 83;

FIG. 87 shows the embodiment of the sliding safety shield illustrated in FIG. 86 inserted into the receiving boot;

FIG. 88 shows a perspective view of an alternative embodiment of a receiving boot operably connected to the shaft of one embodiment of a guiding element;

FIG. 89 shows a bottom perspective view of the embodiment of the receiving boot illustrated in FIG. 88;

FIG. 90 shows a front view of the embodiment of the receiving boot illustrated in FIG. 88 albeit with rails;

FIG. 91 shows a perspective view of the embodiment of the receiving boot illustrated in FIG. 88 and a needle configured with one embodiment of a sliding safety shield;

FIG. 92 shows the embodiment of the sliding safety shield illustrated in FIG. 91 inserted into the receiving boot;

FIG. 93 shows one embodiment of a syringe with an alternative embodiment of a sliding safety shield that is integrally configured to the syringe, approaching one embodiment of a guiding element;

FIG. 94 shows the embodiments of the syringe and sliding safety shield illustrated in FIG. 93, albeit the sliding safety shield has been retracted to expose the needle;

FIG. 95 shows the embodiment of the needle of the syringe illustrated in FIG. 93 inserted into the guiding element;

FIG. 96 shows a top perspective view of one embodiment of a guiding element;

FIG. 97 shows a bottom perspective view of the embodiment of the guiding element illustrated in FIG. 96;

FIG. 98 shows a right side view, a left side view, a front view, and a rear view of the embodiment of the guiding element illustrated in FIG. 96;

FIG. 99 shows a bottom view of the embodiment of the guiding element illustrated in FIG. 96;

FIG. 100 shows a top view of the embodiment of the guiding element illustrated in FIG. 96;

FIG. 101 shows a top perspective view of an alternative embodiment of a guiding element;

FIG. 102 shows a bottom perspective view of the embodiment of the guiding element illustrated in FIG. 101;

FIG. 103 shows a right side view, a left side view, a front view, and a rear view of the embodiment of the guiding element illustrated in FIG. 101;

FIG. 104 shows a bottom view of the embodiment of the guiding element illustrated in FIG. 101;

FIG. 105 shows a top view of the embodiment of the guiding element illustrated in FIG. 101;

FIG. 106 shows a top perspective view of an alternative embodiment of a guiding element;

FIG. 107 shows a bottom perspective view of the embodiment of the guiding element illustrated in FIG. 106;

FIG. 108 shows a right side view, a left side view, a front view, and a rear view of the embodiment of the guiding element illustrated in FIG. 106;

FIG. 109 shows a bottom view of the embodiment of the guiding element illustrated in FIG. 106;

FIG. 110 shows a top view of the embodiment of the guiding element illustrated in FIG. 106;

FIG. 111 shows a top perspective view of an alternative embodiment of a guiding element;

FIG. 112 shows a bottom perspective view of the embodiment of the guiding element illustrated in FIG. 111;

FIG. 113 shows a right side view, a left side view, a front view, and a rear view of the embodiment of the guiding element illustrated in FIG. 111;

FIG. 114 shows a bottom view of the embodiment of the guiding element illustrated in FIG. 111;

FIG. 115 shows a top view of the embodiment of the guiding element illustrated in FIG. 111;

FIG. 116 shows a top perspective view of one embodiment of a volume limiter;

FIG. 117 shows a bottom perspective view of the embodiment of the volume limiter illustrated in FIG. 116;

FIG. 118 shows a front view of the embodiment of the volume limiter illustrated in FIG. 116;

FIG. 119 shows a rear view of the embodiment of the volume limiter illustrated in FIG. 116;

FIG. 120 shows a right side view of the embodiment of the volume limiter illustrated in FIG. 116;

FIG. 121 shows a left side view of the embodiment of the volume limiter illustrated in FIG. 116;

FIG. 122 shows a top view of the embodiment of the volume limiter illustrated in FIG. 116;

FIG. 123 shows a bottom view of the embodiment of the volume limiter illustrated in FIG. 116;

FIG. 124A shows a bottom perspective view of one embodiment of a volume limiter with a syringe's flange inserted into an opening of the volume limiter;

FIG. 124B shows an alternative bottom perspective view of the embodiment of the volume limiter illustrated in FIG. 124A with the syringe's flange fully inserted into a first flange housing element, with the syringe being resiliently engaged by a retention member;

FIG. 125 shows cross sectional views of embodiments of a volume limiter, a plunger inserted through an opening in the volume limiter, and a syringe fully inserted into a first flange housing element of the volume limiter, with the syringe being engaged by a retention member;

FIG. 126 shows a perspective view of one embodiment of a cruciform plunger;

FIG. 127A shows a top view of one embodiment of a volume limiter and a horizontal cross sectional view of one embodiment of a cruciform plunger inserted through an opening in the volume limiter;

FIG. 127B shows a cross sectional view along lines 4-4 of the embodiments of the volume limiter and the cruciform plunger illustrated in FIG. 127A, albeit FIG. 127B also shows a syringe fully inserted into the volume limiter and the cruciform plunger having been repositioned;

FIG. 128 shows a top perspective view of one embodiment of a volume limiter;

FIG. 129 shows a bottom perspective view of the embodiment of the volume limiter illustrated in FIG. 128;

FIG. 130 shows a front view of the embodiment of the volume limiter illustrated in FIG. 128;

FIG. 131 shows a rear view of the embodiment of the volume limiter illustrated in FIG. 128;

FIG. 132 shows a right side view of the embodiment of the volume limiter illustrated in FIG. 128;

FIG. 133 shows a left side view of the embodiment of the volume limiter illustrated in FIG. 128;

FIG. 134 shows a top view of the embodiment of the volume limiter illustrated in FIG. 128;

FIG. 135 shows a bottom view of the embodiment of the volume limiter illustrated in FIG. 128;

FIG. 136 shows a top perspective view of one embodiment of a volume limiter;

FIG. 137 shows a bottom perspective view of the embodiment of the volume limiter illustrated in FIG. 136;

FIG. 138 shows a front view of the embodiment of the volume limiter illustrated in FIG. 136;

FIG. 139 shows a rear view of the embodiment of the volume limiter illustrated in FIG. 136;

FIG. 140 shows a right side view of the embodiment of the volume limiter illustrated in FIG. 136;

FIG. 141 shows a left side view of the embodiment of the volume limiter illustrated in FIG. 136;

FIG. 142 shows a top view of the embodiment of the volume limiter illustrated in FIG. 136;

FIG. 143 shows a bottom view of the embodiment of the volume limiter illustrated in FIG. 136;

FIG. 144 shows a top perspective view of one embodiment of a volume limiter;

FIG. 145 shows a bottom perspective view of the embodiment of the volume limiter illustrated in FIG. 144;

FIG. 146 shows a front view of the embodiment of the volume limiter illustrated in FIG. 144;

FIG. 147 shows a rear view of the embodiment of the volume limiter illustrated in FIG. 144;

FIG. 148 shows a right side view of the embodiment of the volume limiter illustrated in FIG. 144;

FIG. 149 shows a left side view of the embodiment of the volume limiter illustrated in FIG. 144;

FIG. 150 shows a top view of the embodiment of the volume limiter illustrated in FIG. 144;

FIG. 151 shows a bottom view of the embodiment of the volume limiter illustrated in FIG. 144;

FIG. 152 shows a top perspective view of one embodiment of a volume limiter;

FIG. 153 shows a bottom perspective view of the embodiment of the volume limiter illustrated in FIG. 152;

FIG. 154 shows a front view of the embodiment of the volume limiter illustrated in FIG. 152;

FIG. 155 shows a right view of the embodiment of the volume limiter illustrated in FIG. 152;

FIG. 156 shows a rear side view of the embodiment of the volume limiter illustrated in FIG. 152;

FIG. 157 shows a left side view of the embodiment of the volume limiter illustrated in FIG. 152;

FIG. 158 shows a top view of the embodiment of the volume limiter illustrated in FIG. 152;

FIG. 159 shows a bottom view of the embodiment of the volume limiter illustrated in FIG. 152;

FIG. 160 shows a bottom perspective view of one embodiment of a volume limiter;

FIG. 161 shows a top perspective view of the embodiment of the volume limiter illustrated in FIG. 160;

FIG. 162 shows a front view of the embodiment of the volume limiter illustrated in FIG. 160;

FIG. 163 shows a right view of the embodiment of the volume limiter illustrated in FIG. 160;

FIG. 164 shows a rear side view of the embodiment of the volume limiter illustrated in FIG. 160;

FIG. 165 shows a left side view of the embodiment of the volume limiter illustrated in FIG. 160;

FIG. 166 shows a top view of the embodiment of the volume limiter illustrated in FIG. 160;

FIG. 167 shows a bottom view of the embodiment of the volume limiter illustrated in FIG. 160;

FIG. 168 shows a top perspective view of one embodiment of a volume limiter;

FIG. 169 shows a bottom perspective view of the embodiment of the volume limiter illustrated in FIG. 168;

FIG. 170 shows a front view of the embodiment of the volume limiter illustrated in FIG. 168;

FIG. 171 shows a right view of the embodiment of the volume limiter illustrated in FIG. 168;

FIG. 172 shows a rear side view of the embodiment of the volume limiter illustrated in FIG. 168;

FIG. 173 shows a left side view of the embodiment of the volume limiter illustrated in FIG. 168;

FIG. 174 shows a top view of the embodiment of the volume limiter illustrated in FIG. 168;

FIG. 175 shows a bottom view of the embodiment of the volume limiter illustrated in FIG. 168;

FIG. 176 shows a bottom perspective view of one embodiment of a volume limiter;

FIG. 177 shows a top perspective view of the embodiment of the volume limiter illustrated in FIG. 176;

FIG. 178 shows a front view of the embodiment of the volume limiter illustrated in FIG. 176;

FIG. 179 shows a right view of the embodiment of the volume limiter illustrated in FIG. 176;

FIG. 180 shows a rear side view of the embodiment of the volume limiter illustrated in FIG. 176;

FIG. 181 shows a left side view of the embodiment of the volume limiter illustrated in FIG. 176;

FIG. 182 shows a top view of the embodiment of the volume limiter illustrated in FIG. 176; and

FIG. 183 shows a bottom view of the embodiment of the volume limiter illustrated in FIG. 176.

### DETAILED DESCRIPTION

The following description illustrates principles of the disclosure which may be applied in various ways to provide different embodiments. There may be many different forms of embodiments of the disclosure, and as such, embodiments should not be limited to those set forth herein and shown in the accompanying drawings. While exemplary embodiments of the disclosure may be shown and described herein, changes and modifications may be made without departing from its scope and concepts. That which is set forth herein and shown in the accompanying drawings is offered to illustrate the principles of the disclosure and not as limitations. Other variations of the disclosure may be included within the principles of the disclosure.

In some embodiments, the disclosure may be configurable, adaptable and customizable to meet the various needs of various users in various circumstances and to be compatible and used in conjunction with various systems, apparatuses, articles, devices, means, methods and structures. The disclosure may be used for various uses and for various purposes. For example, the disclosure may be used to administer medication to people, animals and things.

The disclosure may be configured in various ways, by various means and various methods, with various parts, to various dimensions (such as shapes, lengths, widths, heights, depths, and sizes) and with and from various materials, and any combinations thereof. The specific parts, materials, members, devices, systems and components of the disclosure may be configured together or separate and with other parts, materials, members, devices, systems and components and any combinations thereof. In one or more embodiments, one or more aspects of the disclosure may be configured in various ways including, for example, but not limited to being configured together or separate, releaseably or non-releaseably.

The drawings herein may but do not necessarily illustrate the disclosure to scale. The drawings herein may but do not necessarily depict the exact positions, shapes, sizes, layouts, designs, angles and other dimensions and configurations in which the disclosure may be implemented. In one or more embodiments, the components of the disclosures may be configured to various positions, shapes, sizes, layouts, designs, angles and other dimensions and configurations from various materials, for various reasons.

At least some components of the disclosure may be formed from various materials. For example, in one or more embodiments, the material used to make the disclosure may include metals (such as aluminum, silver, gold, europium, neptunium, cobalt, iron, cooper, nickel, lead, lithium, calcium, titanium, tin, etc.), non-metals (such as carbon, sulfur, chlorine, argon, etc.), metalloids (such as boron, tellurium, etc.), ceramics (such as alumina, silicon, tungsten, granite, limestone, marble, slate, quartzite, etc.), polymers and plastics (such as natural rubbers, synthetic rubbers, polyvinyl chloride (PVC), PC, high density polyethylene (HDPE), oriented or stretch blown polyethylene terephthalate (PET), polypropylene (PP), acrylonitrile butadiene styrene (ABS), polycarbonate, etc.), alloys (such as alloys of aluminum, alloys of bismuth, alloys of chromium, alloys of cobalt, alloys of copper, alloys of gallium, alloys of gold, alloys of indium, alloys of iron, alloys of lead, alloys of magnesium, alloys of mercury, alloys of nickel, alloys of plutonium, alloys of potassium, rare earth alloys, alloys of rhodium, alloys of scandium, alloys of silver, alloys of sodium, alloys of titanium, alloys of tin, alloys of uranium, alloys of zinc, alloys of zirconium, etc.), woods and natural products (such as hickory, aspen, maple, cedar, spruce, hemlock, pine, oak, walnut, elm, fir, mahogany, kunststoff, cotton, flax, wool, ramie, silk, yarn, denim, corduroy, leather, suede, down, fur, bamboo, jute, etc.), and the like and other materials may be used to make at least some components of the disclosure. In one or more embodiments, components of the disclosure may be made from non-degradable materials (such as glass, stainless steel, titanium, etc.) and degradable or self-dissolving materials (such as sugar and sugar derivatives). In one or more embodiments, components of the disclosure may be made from injection molds.

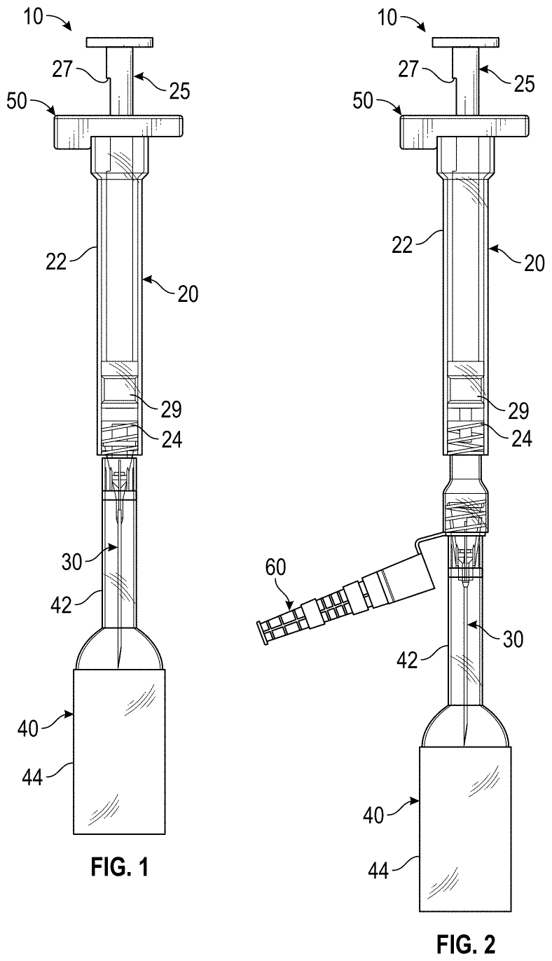

FIG. 1 shows a side view of one embodiment of a medication delivery device 10 which may be designed for the purpose of administering medication. In one or more embodiments, medication delivery device 10 may be configured to facilitate easy and quick access to multi-dose medication vials and a syringe with a fixable volume to ensure proper dosage, prevent overdosing and minimize fine measurements required to be performed by a user. In one or more embodiments, medication delivery device 10 may be designed to provide an inexpensive and flexible alternative to auto-injectors or cartridge-based or other systems while facilitating use and preventing injury to caregivers with limited training, such as family members or patients, as well as trained caregivers in adverse working conditions, such as emergency medical personnel in moving ambulances. In one or more embodiments, medication delivery device 10 may be configured to ensure the correct placement of a needle in a vial, secure the operable connection of the needle and vial while medication is being extracted from the vial, protect the needle from being damaged or causing damage, limit the amount of medication that may be administered, ensure that patients are given the current dosage without having to rely on graduations on the syringe (if any), and ensure that it may be used by individuals with limited training under adverse working conditions.

In one or more embodiments, medication delivery device 10 may be used by various individuals. For example, medication delivery device 10 may be used by a person administering medication to him or her-self or by a person administering medication to another, such as by family members, friends, caretakers and other non-medical professionals, as well as by medical professionals, such as nurses, doctors, dentists, veterinarians and other medical professionals.

In one or more embodiments, medication delivery device 10 may be used in various environments and under various conditions. For example, medication delivery device 10 may be used in emergency medical services, such as in the emergency room or ambulatory setting, the doctor's office, the surgery room, the veterinarian's clinic or on the farm, in the home health services, etc. In one or more embodiments, medication delivery device 10 may be used when the user is hampered by adverse working conditions, including crisis or emergency situations, ambulatory settings, low lighting, bad weather, poor eyesight, unfamiliarity, wearing winter gloves or other clothing or equipment, high-volume vaccination settings, and other conditions that may make it difficult to handle or read or otherwise administer medication.

In one or more embodiments, medication delivery device 10 may be simple and low cost to use and manufacture. In one or more embodiments, medication delivery device 10 may be manufactured for little more than a standard syringe and needle set. In one or more embodiments, medication delivery device 10 may lower the skill level needed to operate it properly, and thereby expand access to inexpensive injectable medications for various uses, such as home care settings. In one or more embodiments, medication delivery device 10 may be designed to remove much of the fine motor skills required under current approaches and thereby make high-volume use more efficient.

In one or more embodiments, medication delivery device 10 may be simple and safe to use and convenient. In one or more embodiments, medication delivery device 10 may be designed such that a user is unlikely to misuse it (such as using it backwards) or spend an unrecoverable dose. In one or more embodiments, medication delivery device 10 may be configured to an array of sizes capable of administering various dosages. In one or more embodiments, at least some components of medication delivery device 10 may be disposable or reusable, single-use or multi-use, or a combination thereof.

In one or more embodiments, medication delivery device 10 may be configured to inject medication into a patient in various locations on the patient's body. For example, medication delivery device 10 may be adapted to administer medication intradermally, intravenously (IV), subcutaneously (SC), intramuscularly (IM), intraperitoneally (IP), intracardially, intraarticularly, intrathecally, and in other locations. In one or more embodiments, medication delivery device 10 may be configured to removed fluid from patients and administer medication orally.

In one or more embodiments, medication delivery device 10 may be used to administer various types of medication. For example, in one or more embodiments, medication delivery device 10 may be used for patients needing injections for vitamin B12, insulin, epinephrine, vaccinations, adenosine or any other injectable medications.

As shown in FIG. 1, in one or more embodiments, medication delivery device 10 may include a syringe 20, a needle 30, a guiding element 40, and a volume limiter 50.

In one or more embodiments, syringe 20 may assume various configurations and be formed from various materials for various purposes, including for the purposes of holding and administering a variety of volumes of medication or other liquids or gases. In one or more embodiments, syringe 20 may be configured to hold microliters, milliliters or liters, or combinations thereof. For example, syringe 20 may hold between about 0.5 μL to about 2 L (such as 1 mL, 3 mL, 5 mL, 10 mL and 20 mL) of medication depending on the level of dosage needed or other desired outcomes. In one or more embodiments, syringe 20 may be designed to be transparent in order to visualize the level of medication. In one or more embodiments, syringe 20 may be designed to be non-transparent. In one or more embodiments, syringe 20 may include graduation markings to indicate the level of medication contained in syringe 20. In one or more embodiments, syringe 20 may be configured in the form of a hypodermic syringe, insulin syringe, microinjector, tuberculin (TB) syringe, LT syringe, TLL syringe, specialty syringes, etc. As shown in FIG. 1, in one or more embodiments, syringe 20 may include various components, such as a flange 21 (not shown in FIG. 1), a syringe barrel 22, a syringe tip 24 and a plunger 25.

In one or more embodiments, flange 21 may assume various configurations and be formed from various materials for various purposes. For example, flange 21 may be configured to include a projecting rim or collar around one end of syringe 20. In one or more embodiments, volume limiter 50 may be operably connected to flange 21 and a user may engage the operably connected volume limiter 50 to aid in drawing, handling and injecting medication into a patient. In one or more embodiments, syringe barrel 22 may assume various configurations and be formed from various materials for various purposes, including for the purposes of intaking, holding and expelling medication or other liquids or gases. For example, syringe barrel 22 may be a cylindrical tube which allows plunger 25 to be slideably pulled and pushed along the inside surface of syringe barrel 22 in order to intake and expel substances. In one or more embodiments, syringe barrel 22 may be made from glass, plastic and/or other materials. In one or more embodiments, syringe barrel 22 may be transparent or non-transparent and may include volume markers. In one or more embodiments, syringe tip 24 may assume various configurations and be formed from various materials for various purposes. In one or more embodiments, syringe tip 24 may be configured as a Luer-Lok tip wherein needle 30 may be twisted into syringe tip 24 for operably connecting syringe 20 to needle 30 or for operably connecting other components or devices (such as safety shield 60 as shown in FIG. 2). Although not shown in FIG. 1, in one or more embodiments, syringe tip 24 may be configured as a slip tip, an eccentric tip (which may be designed for administering medication close to the surface of the skin, such as artery injections or injections into veins), a catheter tip (which may be designed for irrigation purposes or to be used in conjunction with tubing) or other tip configurations. In one or more embodiments, syringe tip 24 may assume other configurations to be used in connection with various devices. In one or more embodiments, plunger 25 may assume various configurations and be formed from various materials for various purposes. For example, plunger 25 may be made from plastic, rubber or other materials or combinations thereof for the purposes of drawing and expelling medication or other substances into and out of syringe barrel 22. In one or more embodiments, plunger 25 may be rebated, notched, grooved, angled, protruded, channeled, serrated, flattened, rounded, cruciformed, modified, shaped or otherwise configured at different locations or depths in order to accommodate different volumes. In one or more embodiments, a color coding system on plunger 25 and other components of medication delivery device 10 may be used. In one or more embodiments, plunger 25 may include a plunger stopper 29 for forming a tight seal between plunger 25 and syringe barrel 22. In one or more embodiments, plunger stopper 29 may assume various configurations and be formed from various materials for various purposes. For example, plunger stopper 29 may be made from plastic, rubber or other materials or combinations thereof.

In one or more embodiments, needle 30 may assume various configurations and be formed from various materials for various purposes. For example, in one or more embodiments, needle 30 may be configured to various thicknesses, gauges and lengths dependent on anticipated injection sites, the amount and viscosity of medication to be administered, and other factors. In one or more embodiments, needle 30 may be about a 2 gauge to about a 36-gauge needle. In one or more embodiments, needle 30 may be about ¼ of an inch to about 8 inches in length. In one or more embodiments, needle 30 may be configured as a microneedle, arrays of needles, a specialty needle or other needle types. In one or more embodiments, needle 30 may be designed to reach nearly to the bottom of an upright vial 70 or to penetrate just the septum of an inverted vial 70. In one or more embodiments, a shield or other device or means may shield needle 30 to prevent injury or damage to needle 30 and to prevent needle sticks. More will be discussed about needle 30 below.

In one or more embodiments, guiding element 40 may assume various configurations and be formed from various materials for various purposes. For example, in one or more embodiments, guiding element 40 may include a guiding element shaft 42 for housing and protecting needle 30 and a guiding element barrel 44 for securing or otherwise engaging a vial 70 and guiding needle 30 into substantially the center of vial 70. In one or more embodiments, guiding element 40 may limit the depth to which needle 30 may be inserted into vial 70. In one or more embodiments, guiding element 40 may include a removable sleeve (such as guiding element shaft 42) to set the depth needle 30 may travel into vial 70. In one or more embodiments, guiding element shaft 42 may shield needle 30 in order to reduce or eliminate the risk of needle 30 being damaged or causing damage to users or others, or of being contaminated. In one or more embodiments, guiding element 40 may keep needle 30 from being blunted or inadvertently bent. In one or more embodiments, guiding element 40 may shield needle 30 to prevent accidental sticks by extending past the tip of needle 30. In one or more embodiments, guiding element 40 may assume a cup-like shape attached to the end of a cut-down needle shield. In one or more embodiments, guiding element 40 may replace a standard needle shield with a configuration that may be opened and expanded on one end to a diameter that allows vial 70 to be placed into it. In one or more embodiments, the expanded area of guiding element 40 may be minimized or reduced in order to stop vial 70 at a point that allows the bevel of needle 30 to pass through the septum such that it remains submerged in the medication and is kept from contacting the sides of vial 70. In one or more embodiments, guiding element 40 may be designed to shield the user from broken glass, such as when a user uses a glass vial 70 wherein special filtered needles may be required. In one or more embodiments, guiding element 40 may or may not contain a venting needle 200 or a venting channel 201 to prevent vacuum. In one or more embodiments, guiding element 40 may be removed after use in order to expose needle 30 and allow a user to inject a patient. In one or more embodiments, guiding element 40 may use the same needle 30 for injections as well as access vial 70 and therefore require no needle change before injections, thereby eliminating the need to manually attach needle 30 after using the guiding element 40 as needle 30 may be pre-attached. In one or more embodiments, guiding element 40 may use inserts, a sliding shield, springs, rollers, semi-flexible material, or other materials and configurations to accept varying sizes of vials 70. In one or more embodiments, guiding element 40 may be made from rubber, plastic, metal or other materials, or combinations thereof. More will be discussed about guiding element 40 below.

In one or more embodiments, volume limiter 50 may assume various configurations and be formed from various materials for various purposes, including for the purpose of limiting the amount of medication that syringe 20 can draw from vial 70. In one or more embodiments, volume limiter 50 may be a single piece or device. In one or more embodiments, volume limiter 50 may include multiple components. In one or more embodiments, volume limiter 50 may include at least one component that may be moveable relative to other volume limiter 50 components or other components of medication delivery device 10. In one or more embodiments, volume limiter 50 may be designed as a slideable clip. In one or more embodiments, volume limiter 50 may be a slideable clip attachable to flange 21 of syringe 20, which syringe 20 may include a plunger 25 that may be rebated, notched, grooved, angled, protruded, channeled, serrated, flattened, rounded, cruciformed, modified, shaped or otherwise configured, though other methods (such as pins, detachable clips, and stops that engage plunger 25) may be used. In one or more embodiments, volume limiter 50 may include a rectangular piece configured to slide across the top of syringe 20 (such as flange 21) and a flat sided or otherwise shaped hole (such as an oblong hole) to engage a rebated, notched, grooved, angled, protruded, channeled, serrated, flattened, rounded, cruciformed, modified, shaped or otherwise configured section of plunger 25 (such as engagement section 26). In one or more embodiments, volume limiter 50 may automatically engage and include a spring to slide volume limiter 50 once the engagement section 26 of plunger 25 travels a certain distance. In one or more embodiments, volume limiter 50 may be adjustable to allow various dosages to be administered. In one or more embodiments, volume limiter 50 may be formed as a single clip design used for all volumes. In one or more embodiments, volume limiter 50 may be fixed or disengageable to allow for draw-back to check for “flash” upon injection. More will be discussed about volume limiter 50 below.

Although not shown in FIG. 1, medication delivery device 10 may include other components. For example, needle 30 may include a needle cap, cover, shield, sleeve, stop or safety cap for the purpose of covering the exposed portion of needle 30. In one or more embodiments, the disclosure may be light weight and compact. In one or more embodiments, medication delivery device 10 may be about 10 grams to about 15 grams in weight and about 6 inches to about 8 inches in length when fully assembled. In one or more embodiments, syringe 20 may be about 4 grams to about 6 grams in weight and about 4 inches to about 5.5 inches in length. In one or more embodiments, guiding element 40 may be about 4 grams to about 6 grams in weight and about 1.5 inches to about 2.5 inches in length. In one or more embodiments, volume limiter 50 may be about 1 gram to about 3 grams in weight, about 1 inch to about 1.5 inches in length and about 0.375 inch to about 0.5 inch in width, and about 0.15 inch to about 0.3 inch in depth.

FIG. 2 shows a side view of one embodiment of medication delivery device 10 including syringe 20, needle 30, guiding element 40, volume limiter 50 and a safety shield 60. In one or more embodiments, needle 30 may be operably connected to safety shield 60 and safety shield 60 may be operably connected to syringe tip 24. In one or more embodiments, safety shield 60 may be configured to be articulatable relative to needle 30. In one or more embodiments, after administering medication, safety shield 60 may be designed to cover needle 30 in order to prevent further sticking, the spread of disease, and for other reasons. Although FIG. 2 shows safety shield 60 configured as a Protex Needle-Pro® device, in one or more embodiments, safety shield 60 may assume various configurations and be formed from various materials. In one or more embodiments, safety shield 60 may be sold or otherwise provided separately.

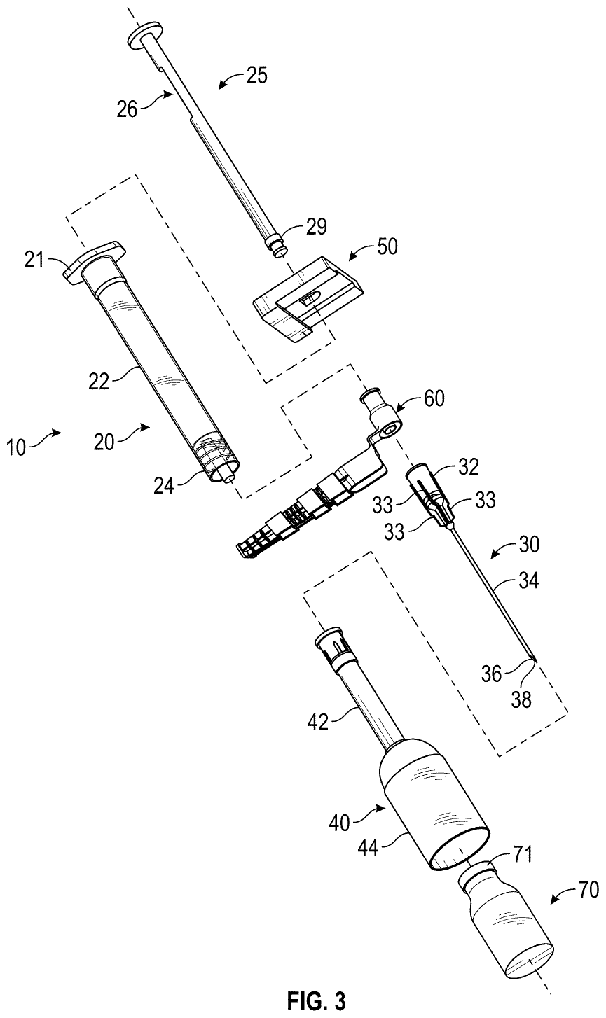

FIG. 3 shows an exploded perspective view of one embodiment of various components of medication delivery device 10 including syringe 20, needle 30, guiding element 40, volume limiter 50 and safety shield 60. In one or more embodiments, medication delivery device 10 may provide a means for guiding needle 30 into substantially the center of vial 70 and for securing or otherwise engaging vial 70 while a user draws medication from it. For example, as indicated above, in one or more embodiments, guiding element 40 may include guiding element shaft 42 for housing and protecting needle 30 and guiding element barrel 44 for securing or otherwise engaging vial 70 and guiding needle 30 into substantially the center of vial 70. In one or more embodiments, vial 70 may be inserted into guiding element 40 which is configured to secure or otherwise engage vial 70 while medication is extracted from vial 70 into syringe 20. In one or more embodiments, once the medication has been extracted, guiding element 40 may be removed from medication delivery device 10 and the medication may be administered to the patient. In one or more embodiments, vial 70 may include a cap 71 which may include a rubber septum or stopper or some other object or material through which needle 30 may be inserted to draw medication. In one or more embodiments, medication delivery device 10 may be compatible with standard multi-dose vials in widespread production. In one or more embodiments, medication delivery device 10 may be compatible with vials of various configurations and sizes and made from various materials (including glass, plastic and other materials). For example, vial 70 may be configured as a screw vial, lip vial, crimp vial, snap-top vial, hinged-capped vial or other configuration. As shown in FIG. 3, in one or more embodiments, needle 30 may include a hub 32, a shaft 34, a lumen 36 and a bevel 38. In one or more embodiments, the various components of medication delivery device 10 may be sold, manufactured or otherwise provided separately or operably connected together or a combination thereof. For example, syringe 20, needle 30 and safety shield 60 may be sold, manufactured or otherwise provided operably connected together, with guiding element 40 and volume limiter 50 provided unconnected. Alternatively and/or in addition, syringe 20, needle 30, guiding element 40, volume limiter 50 and safety shield 60 may be sold, manufactured or otherwise provided all operably connected together (as shown in FIG. 2). Alternatively and/or in addition, various other combinations of the connectivity of various components of medication delivery device 10, including other devices not shown herein, may be sold, manufactured or otherwise provided separately and/or operably connected together.

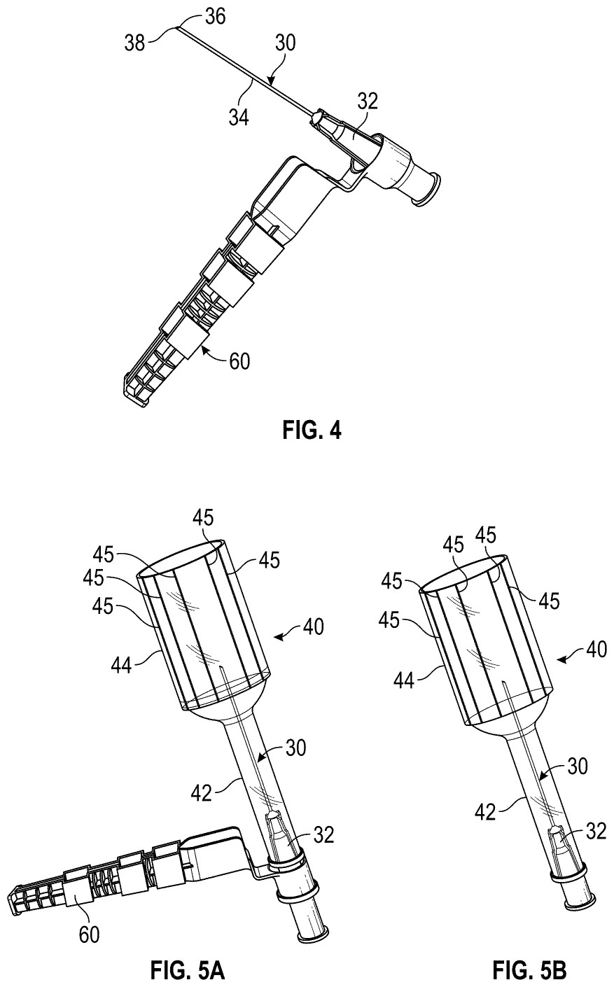

FIG. 4 shows a perspective view of one embodiment of needle 30 operably connected to one embodiment of safety shield 60. As shown in FIG. 4, and as previously indicated, needle 30 may include hub 32, shaft 34, lumen 36 and bevel 38. In one or more embodiments, hub 32 may assume various configurations and be formed from various materials for various purposes. For example, hub 32 may be configured as a Luer-Lok hub for engaging with a corresponding element of safety shield 60. In one or more embodiments, a Luer-Lok hub 32 configuration may provide an unacceptable volume of dead space leading to an about 0.1 mL shortage when a dose is drawn. To remedy such an issue, in one or more embodiments, needle 30 may be integral to syringe tip 24 to reduce the volume to a negligible level. In one or more embodiments, hub 32 may be configured to engage syringe tip 24 directly. For example, as shown in FIG. 1, hub 32 may be configured as a Luer-Lok hub for engaging with a corresponding Luer-Lok syringe tip 24. Although not shown in FIG. 4, in one or more embodiments, hub 32 may be configured as a slip hub, metal hub, Kel-F hub, or some other hub configurations. In one or more embodiments, hub 32 may include various materials (such as polypropylene) which may aid in performing hub's 32 desired function. In one or more embodiments, lumen 36 may define the diameter of the hole or gauge of needle 30 and bevel 38 may define that angle at which shaft 34 may be configured.

FIG. 5A shows a perspective view of one embodiment of guiding element 40 operably connected to and housing one embodiment of needle 30, which needle 30 is operably connected to one embodiment of safety shield 60. In one or more embodiments, once a user draws medication from vial 70, the user may remove guiding element 40, inject the medication into the patient, and then, optionally, articulate safety shield 60 and cover needle 30. Alternatively and/or in addition, in one or more embodiments, once a user draws medication from vial 70, the user may remove guiding element 40, inject the medication into the patient, and then, optionally, place guiding element 40 back over needle 30 for reuse, to cover needle 30 or for some other purpose.

FIG. 5B shows a perspective view of one embodiment of guiding element 40 operably connected to one embodiment of needle 30 without the presence of safety shield 60. In one or more embodiments, once a user draws medication from vial 70, the user may remove guiding element 40, inject the medication into the patient, and then, optionally, place guiding element 40 back over needle 30 for reuse, to cover needle 30 or for some other purpose. As shown in FIG. 5B and elsewhere, guiding element barrel 44 may be configured with ribs 45 which may stabilize and reinforce the strength of guiding element barrel 44 and aid in its ability to resiliently secure vial 70. Although not shown in FIG. 5B, in one or more embodiments, guiding element barrel 44 may be configured without ribs 45 (such as shown in FIGS. 1 and 2). In one or more embodiments, ribs 45 may be configured substantially horizontally (instead of or in addition to substantially vertically as shown in FIG. 5B) on guiding element barrel 44.

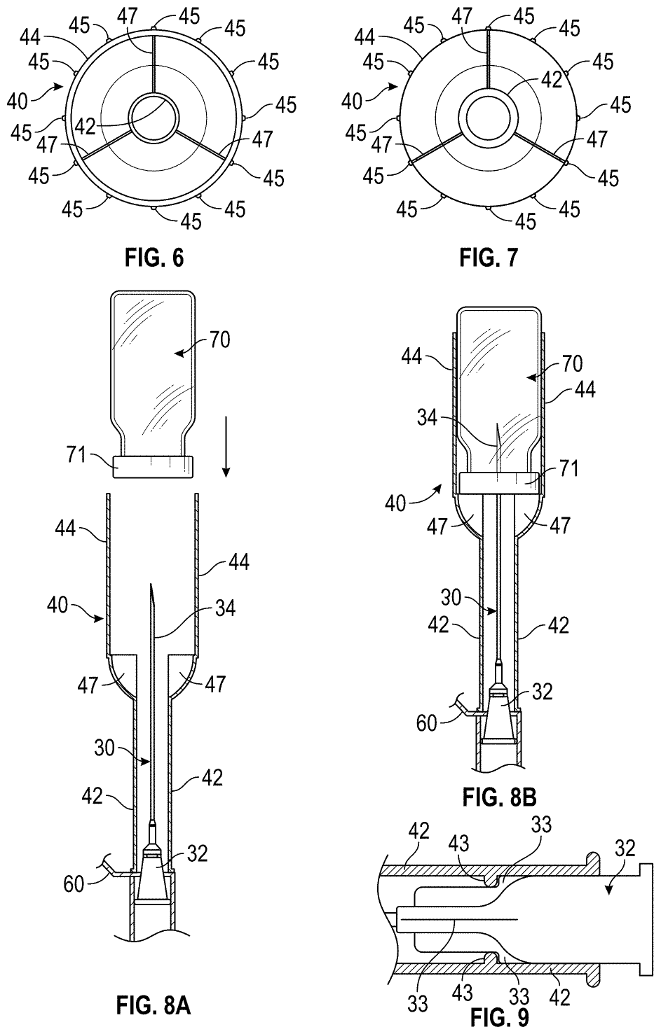

FIG. 6 shows a bottom view of one embodiment of guiding element 40. In one or more embodiments, the thickness of guiding element barrel 44 may vary and be adaptable for securing or otherwise engaging vial 70.

FIG. 7 shows a top view of the embodiment of guiding element 40 illustrated in FIG. 6.

FIG. 8A shows a cross sectional view of one embodiment of guiding element 40, along with a side view of one embodiment of needle 30 and one embodiment of vial 70, wherein vial 70 is approaching guiding element 40. As shown in FIG. 8A, guiding element 40 may include at least three wings 47 (only two of the at least three wings 47 are shown in FIG. 8A) which may be designed to engage cap 71 of vial 70 and control the depth at which needle 30 accesses vial 70.

FIG. 8B shows a cross sectional view of the embodiment of guiding element 40 illustrated in FIG. 8A, albeit vial 70 has been inserted into guiding element barrel 44 and needle 30 has punctured the rubber septum of cap 71 and is being secured or otherwise engaged by guiding element barrel 44 in order to allow a user to draw medication from vial 70. In one or more embodiments, such configuration may be used to secure vial 70 and needle 30 below the medication level and reduce or eliminate the need for fine motor skills or other skills while administering medication under various working conditions, including adverse working conditions. As shown in FIG. 8B, the at least three wings 47 may be configured to contact cap 71 of vial 70 and control the depth at which needle 30 may access vial 70. In one or more embodiments, the at least three wings 47 may aid in stabilizing the position of vial 70 while it is secured in the guiding element barrel 44. In one or more embodiments, guiding element barrel 44 may be configured such that it applies force on, grips, contracts, secures, or otherwise engages vial 70. In one or more embodiments, guiding element barrel 44 may be configured such that it positions or guides needle 30 into substantially the center of cap 71, thereby reducing the risk of bending, blunting, breaking or otherwise damaging needle 30. In one or more embodiments, once a user extracts medication from vial 70, the user may simply remove the guiding element 40 (which may be securing vial 70), expose needle 30 and inject the patient. Such configuration eliminates the step of requiring the end-user to attach needle 30 to syringe 20 after using guiding element 40 to extract the medication from vial 70 (because needle 30, in one or more embodiments, may already be attached to syringe 20 prior to accessing vial 70).

FIG. 9 shows a cross sectional view of one embodiment of aspects of guiding element shaft 42, along with a side view of one embodiment of hub 32 of needle 30. In one or more embodiments, needle 30 may be housed within guiding element 40 in a substantially temporary fixed position while vial 70 is inserted into guiding element 40. In one or more embodiments, such an arrangement may allow a user to quickly and accurately insert needle 30 in the center of the vial 70 when vial 70 is inserted into guiding element 40. In one or more embodiments, guiding element 40 and needle 30 may be configured in various ways to accomplish the desired quickness and accuracy. For example, as shown in FIG. 9, in one or more embodiments, hub 32 may include wings 33 which may aid guiding element 40 in being operably connected to needle 30. In one or more embodiments, wings 33 may engage the inner wall of some aspect of guiding element 40 (such as guiding element shaft 42) and apply force on or engage said inner wall, thereby securing needle 30 in a substantially temporary fixed position within guiding element 40. As shown in FIG. 9, in one or more embodiments, the inner wall of guiding element shaft 42 may include a protrusion 43 which may be positioned on or circumference the inner wall, which when engaged by wings 33, may limit the distance needle 30 may travel into guiding element shaft 42.

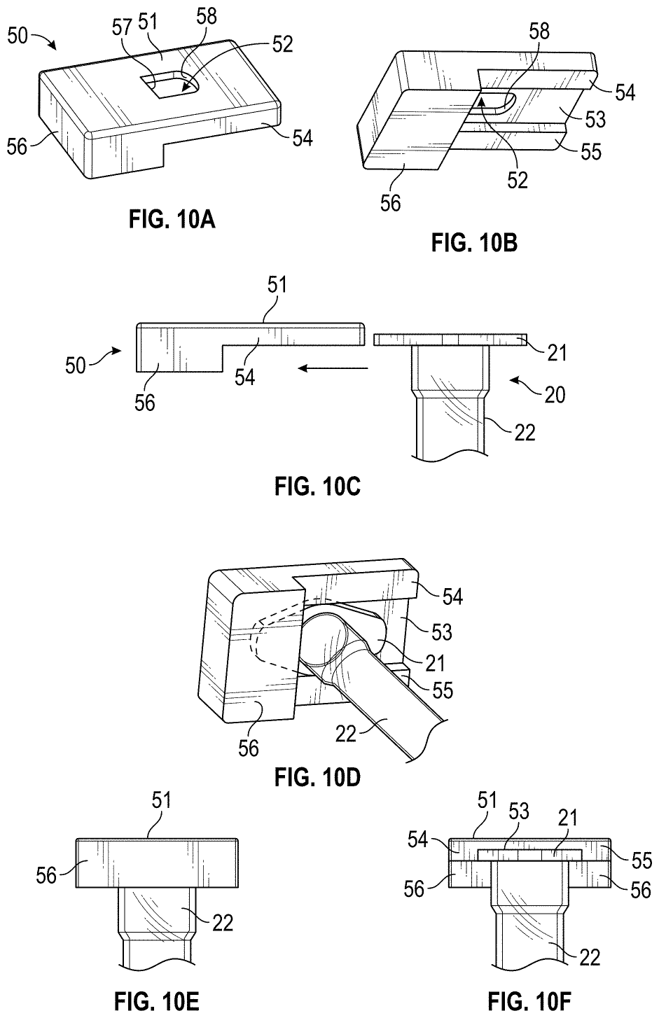

FIGS. 10A-10F show various views of one embodiment of volume limiter 50.

FIG. 10A shows a top perspective view of one embodiment of volume limiter 50. In one or more embodiments, medication delivery device 10 may provide a means for limiting the amount of medication that can be administered to a patient. For example, in one or more embodiments, volume limiter 50 may be configured to operably connect to syringe 20 and limit the amount of medication syringe 20 can draw from vial 70, thereby limiting the amount of medication that can be administered to a patient, preventing overdosing, and ensuring that the correct dosage is administered. In one or more embodiments, as shown in FIG. 10A, volume limiter 50 may include a top surface 51, an opening 52 through which plunger 25 may be inserted, a bottom surface 53 (not shown in FIG. 10A), a first bottom ledge 54, a second bottom ledge 55 (not shown in FIG. 10A), and a first flange housing element 56. In one or more embodiments, as shown in FIG. 10A, opening 52 may include a first surface 57 (which may assume the contour of the engagement section 26 of plunger 25) and a second surface 58 (which may assume an alternative contour). As shown in FIG. 10A, first surface 57 may be configured to be flat in order to conform to a notched or rebated engagement section 26. As shown in FIG. 10A, second surface 58 may be configured to be rounded.

FIG. 10B shows a bottom perspective view of the embodiment of volume limiter 50 illustrated in FIG. 10A.

FIG. 10C shows a side view of the embodiment of volume limiter 50 illustrated in FIG. 10A, as well as an approaching embodiment of syringe 20. In one or more embodiments, first bottom ledge 54 and second bottom ledge 55 may be configured to allow flange 21 of syringe 20 to slide in between the two ledges and be secured by the same. In one or more embodiments, first flange housing element 56 may be configured to allow flange 21 of syringe 20 to slide into and be secured by it. Although not shown in FIG. 10C, in one or more embodiments, volume limiter 50 may be configured with various protrusions, ridges, indentations, or other configurations or materials, or combinations thereof, which may aid in securing syringe 20 to volume limiter 50. In one or more embodiments, the securement of syringe 20 to volume limiter 50 may be temporary, permanent, releasable, adjustable, resilient or otherwise engaged or any combination thereof.

FIG. 10D shows a bottom perspective view of the embodiment of volume limiter 50 illustrated in FIG. 10A, wherein flange 21 of syringe 20 has been inserted in between first bottom ledge 54 and second bottom ledge 55 and positioned into first flange housing element 56.

FIG. 10E shows a back view of the embodiment of volume limiter 50 illustrated in FIG. 10A, wherein flange 21 of syringe 20 has been inserted in between first bottom ledge 54 and second bottom ledge 55 and positioned into first flange housing element 56.

FIG. 10F shows a front view of the embodiment of volume limiter 50 illustrated in FIG. 10A, wherein flange 21 of syringe 20 has been inserted in between first bottom ledge 54 and second bottom ledge 55 and positioned into first flange housing element 56.

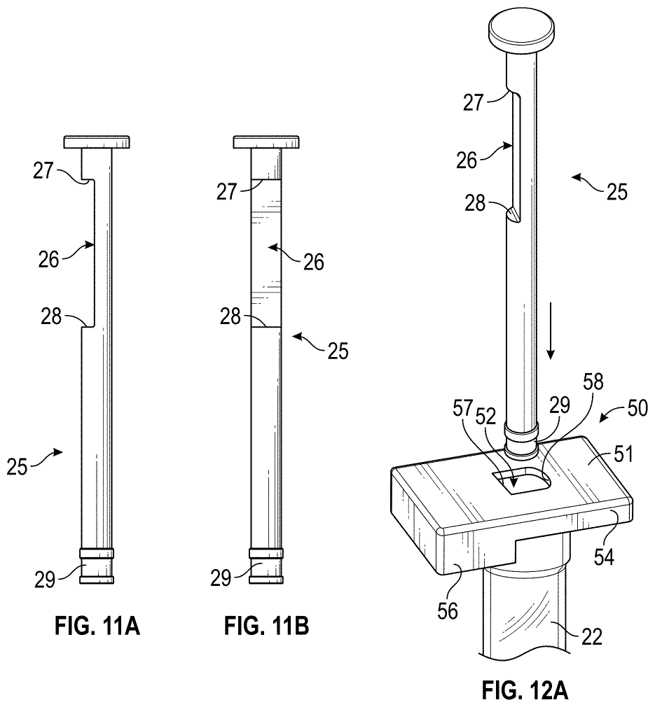

FIG. 11A shows a side view of one embodiment of plunger 25. As shown in FIG. 11A, plunger 25 may include an engagement section 26 for limiting the distance the plunger can travel within syringe barrel 22 to prevent overdosing and ensuring that the correct dosage is administered. As shown in FIG. 11A, engagement section 26 may be notched or rebated and include a first end 27 and a second end 28.

FIG. 11B shows a front view of the embodiment of plunger 25 illustrated in FIG. 11A.

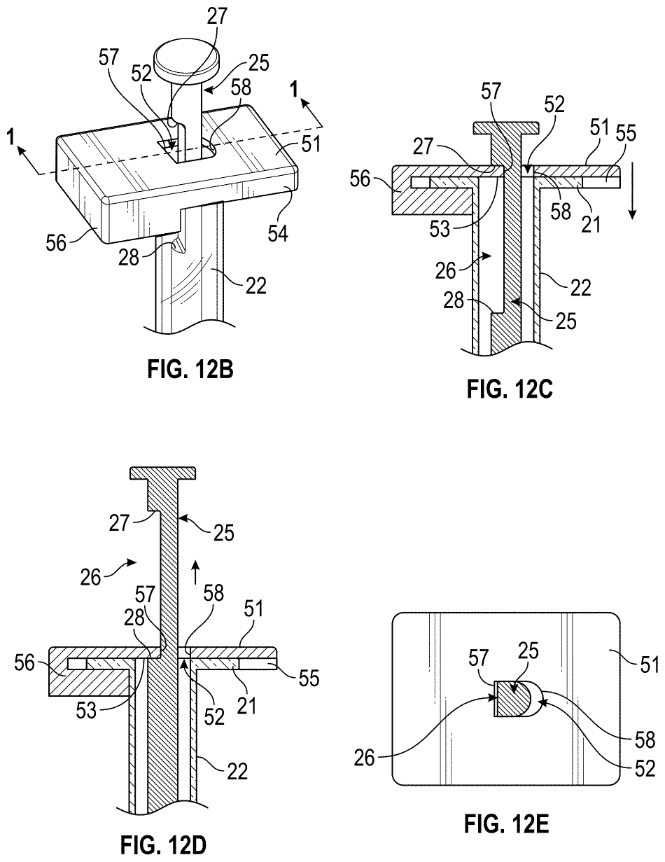

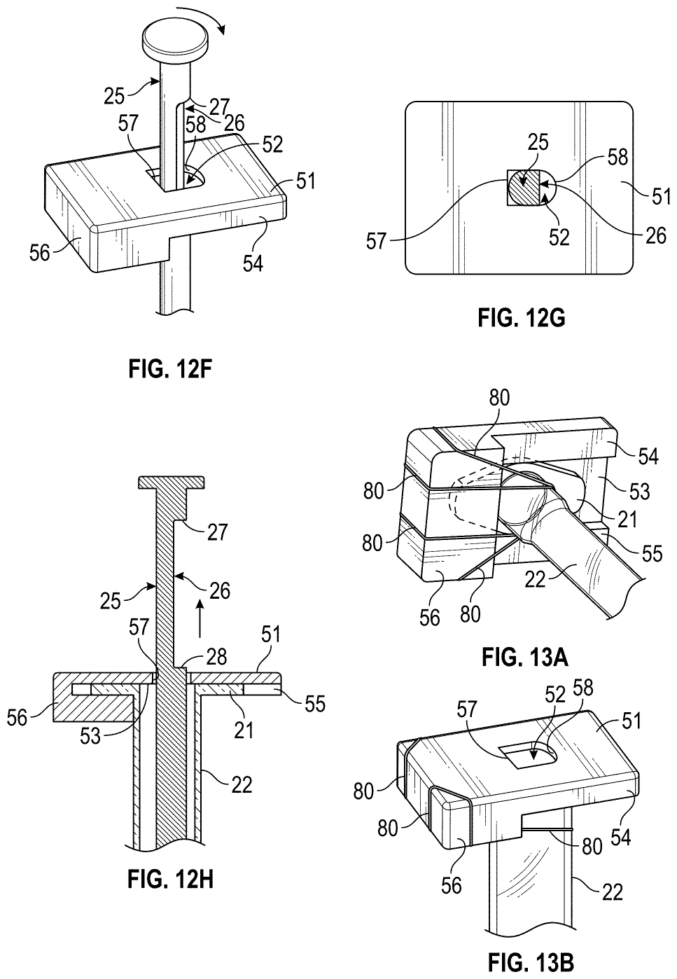

FIGS. 12A-12H show various views of the embodiment of plunger 25 illustrated in FIG. 11A being engaged with the embodiment of volume limiter 50 illustrated in FIG. 10A.

FIG. 12A shows a perspective view of the embodiment of plunger 25 illustrated in FIG. 11A approaching the embodiment of volume limiter 50 illustrated in FIG. 10A. As shown in FIG. 12A, one embodiment of syringe 20 may be operably connected to volume limiter 50 (such as the volume limiter 50 illustrated and described in relation to FIGS. 10A-10F).

FIG. 12B shows a perspective view of the embodiment of plunger 25 illustrated in FIG. 11A inserted through opening 52 of the embodiment of volume limiter 50 illustrated in FIG. 10A, with syringe 20 operably connected to volume limiter 50. As shown in FIG. 12B, plunger 25 may be positioned such that engagement section 26 is proximal to first surface 57 and that first end 27 is located on one side of volume limiter 50 and second end 28 is located on another side of volume limiter 50.

FIG. 12C shows a cross sectional view along lines 1-1 of the embodiments of plunger 25, volume limiter 50 and syringe 20 illustrated in FIG. 12B, albeit plunger 25 has been depressed and moved such that engagement section 26 has engaged first surface 57. As shown in FIG. 12C, in one or more embodiments, plunger 25 may be configured such that once plunger 25 is inserted through opening 52, plunger 25 may travel within syringe barrel 22 until first end 27 engages the top surface 51 of volume limiter 50 or until plunger stopper 29 hits the bottom of syringe barrel 22. In one or more embodiments, once first end 27 of plunger 25 engages the top surface 51 of volume limiter 50 or plunger stopper 29 hits the bottom of syringe barrel 22, a user may be prevented from expelling substances (including liquid and air) from syringe 20 in a particular depression. Such configuration may be at least one way medication delivery device 10 may provide for limiting the amount of medication that can be injected into a patient and for limiting the amount of air a user may force into vial 70.

FIG. 12D shows cross sectional views along lines 1-1 of the embodiments of plunger 25, volume limiter 50 and syringe 20 illustrated in FIG. 12B, albeit that plunger 20 has been raised. As shown in FIG. 12D, in one or more embodiments, if the engagement section 26 is aligned with the first surface 57 of opening 52, plunger 25 may travel within syringe barrel 22 until second end 28 engages the bottom surface 53 of volume limiter 50. Such configuration may be at least one way medication delivery device 10 may provide for limiting the amount of medication that can be drawn into syringe barrel 22 from vial 70 and thereby limit the amount of medication that can be injected into a patient, prevent overdosing, and ensure that the correct dosage is administered.

FIG. 12E shows a top view of the embodiment of volume limiter 50 illustrated in FIG. 10A and a horizontal cross sectional view at the engagement section 26 of the embodiment of plunger 25 illustrated in FIG. 11A, which plunger 25 has been inserted through the opening 52 of volume limiter 50 and engaging engagement section 26 is facing or aligned with first surface 57 of opening 52. In one or more embodiments, first surface 57 may engage engagement section 26 as plunger 25 travels within syringe 20.

FIG. 12F shows a perspective view of the embodiment of plunger 25 illustrated in FIG. 11A inserted through opening 52 of volume limiter 50 and twisted to disengage volume limiter 50 from plunger 25 and allow plunger 25 to travel further to check for “flash” or for other reasons. FIG. 12F shows the embodiment of volume limiter 50 illustrated in FIG. 10A. As shown in FIG. 12F, plunger 25 may be twisted such that engagement section 26 does not catch on first surface 27. Such configuration may provide a means for disengaging volume limiter 50 from plunger 25 to allow plunger 25 to travel further. In one or more embodiments, plunger 25 may be twisted to about 180 degrees (or so that the movement of second end 28 is no longer hindered by bottom surface 53) to disengage volume limiter 50 from plunger 25 and allow plunger 25 to travel further. In one or more embodiments, alternative or additional means may be provided for disengaging volume limiter 50 from plunger 25 or some other aspect of medication delivery device 10 to allow plunger 25 to travel further. For example, although not shown in FIG. 12F, in one or more embodiments, volume limiter 50 may be designed to allow a user to shift or otherwise reposition volume limiter 50 in order to disengage it from plunger 25 or some other component of medication delivery device 10 (such as shifting first surface 57 so that it disengages engagement section 26 of plunger 25), thereby allowing plunger 25 to travel further. Such configuration(s) may be in addition to or alternative to being able to twist plunger 25 to disengage the volume limiter 50.

FIG. 12G shows a top view of the embodiment of volume limiter 50 illustrated in FIG. 10A and a horizontal cross sectional view at the engagement section 26 of the embodiment of plunger 25 illustrated in FIG. 11A, which plunger 25 has been inserted through opening 52 of volume limiter 50 and twisted such that engagement section 26 is proximal to second surface 58, thereby disengaging volume limiter 50 from plunger 25.

FIG. 12H shows a cross sectional view along lines 1-1 of the embodiments of plunger 25, volume limiter 50 and syringe 20 illustrated in FIG. 12B, albeit plunger 25 has been twisted (as shown in FIG. 12G) and raised. As shown in FIG. 12H, in one or more embodiments, once plunger 25 may be twisted so that engagement section 26 is not proximal to first surface 57, plunger 25 may travel within syringe barrel 22 at a greater distance because engagement section 26 does not catch on bottom surface 53. In one or more embodiments, such an arrangement may allow a user, who has already inserted needle 30 into a patient, to draw back and check for blood or extract substances from a patient. Although not shown in FIG. 12H, in one or more embodiments, after twisting plunger 25 or otherwise disengaging volume limiter 50 from engagement section 26, plunger 25 may be removed through opening 52 and separated from medication delivery device 10. Although not shown in FIG. 12H, in one or more embodiments, twisting plunger 25 may cause volume limiter 50 to shift (such as substantially perpendicularly to plunger 25) to aid in allowing plunger 25 to travel further.

FIGS. 13A-13B show various views of one embodiment of volume limiter 50 including a retention member 80.

FIG. 13A shows a bottom perspective view of the embodiment of volume limiter 50 illustrated in FIG. 10D, albeit the volume limiter 50 shown in FIG. 13A includes a retention member 80. In one or more embodiments, medication delivery device 10 may provide a means for at least temporarily and resiliently securing the operable connection of volume limiter 50 with syringe 20. For example, in one or more embodiments, retention member 80 may be a rubber band or some other material that may be stretched around various components of medication delivery device 10 in order to temporarily and resiliently secure volume limiter 50 with syringe 20. As shown in FIG. 13A, in one or more embodiments, retention member 80 may be positioned around one side of syringe 20 and the opposite corners of first flange housing element 56 and top surface 51 such that syringe 20 is temporarily and resiliently placed in a fixed position relative to volume limiter 50. In one or more embodiments, as plunger 25 is twisted or otherwise moved relative to volume limiter 50, volume limiter 50 may resiliently shift positions or slide along flange 21. In one or more embodiments, medication delivery device 10 may provide a means for automatically engaging volume limiter 50 for limiting the amount of medication that can be drawn from vial 70, and thereby limit the amount of medication that can be administered to a patient. For example, in one or more embodiments, retention member 80 may enable volume limiter 50 to automatically engage plunger 25 when plunger 25 is depressed through opening 52. In one or more embodiments, as plunger 25 is inserted and depressed through opening 52 (such as shown in FIGS. 12A-12C) with engagement section 26 aligned with the first surface 57 of opening 52, retention member 80 may enable volume limiter 50 to automatically adjust to the contour of and apply force on the surface of engagement section 26. In one or more embodiments, once second end 28 travels through opening 52 and past bottom surface 53, retention member 80 may enable volume limiter 50 to automatically snap against and apply force on engagement section 26, and thereby limit the distance plunger 25 may travel to the distance between first end 27 and second end 28 (as shown in FIGS. 12C and 12D). Such means for automatically engaging volume limiter 50 may, in one or more embodiments, allow plunger 25 to be provided to a user in a drawn position with second end 28 positioned against or proximal to bottom surface 53 ready for the user to (1) insert an attached needle 30 into vial 70 with the aid of guiding element 40, (2) depress plunger 25 and force air into vial 70, and (3) let pressure force plunger 25 back and fill syringe 20 with medication from vial 70 up to the point at which second end 28 engages bottom surface 53. Such configuration may limit the amount of medication that can be drawn from vial 70 and thereby limit the amount of medication that can be administered to a patient, prevent overdosing, and ensure that the correct dosage is administered.

FIG. 13B shows a top perspective view of the embodiment of volume limiter 50 illustrated in FIG. 13A including a retention member 80.

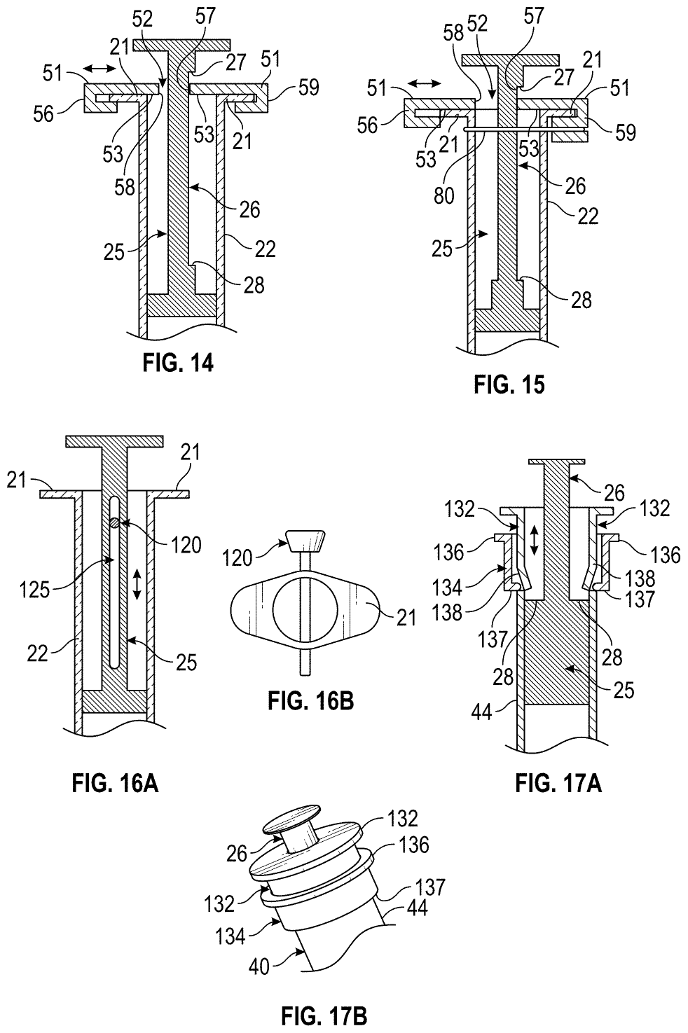

FIGS. 14-26E show views of various embodiments of volume limiter 50, plunger 25 and syringe 20 that may, in one or more embodiments, be mixed and matched and substituted with other embodiments of volume limiter 50, plunger 25 and syringe 20 contained herein.

FIG. 14 shows a cross sectional view of one embodiment of volume limiter 50 which may be operably connected to syringe 20 such that it may not be removed. As shown in FIG. 14, volume limiter 50 may include first flange housing element 56 and a second flange housing element 59, as well as top surface 51, opening 52 (which may include first surface 57 and second surface 58) and bottom surface 53. In one or more embodiments, first flange housing element 56 and second flange housing element 59 engage at least aspects of flange 21 such that volume limiter 50 may not be removed from syringe 20. For example, second flange housing element 59 may be configured similar to the first flange housing element 56 illustrated in FIGS. 10A through 10F, albeit second flange housing element 59 may be located on the opposite side of volume limiter 50. In one or more embodiments, first surface 57 of opening 52 may be configured so that first surface 57 engages engagement section 26 of plunger 25 in order to limit the distance plunger 25 may travel between second end 28 and first end 27, and thereby limit the amount of medication that can be drawn from vial 70 which will limit the amount of medication that can be administered to a patient, prevent overdosing, and ensure that the correct dosage is administered. In one or more embodiments, volume limiter 50 may be manually repositioned, shifted or otherwise moved or operated (such as substantially perpendicularly in relation to plunger 25) by a user in order to move first surface 57 away from engagement section 26 and disengage volume limiter 50 from plunger 25. Such configuration may be one embodiment of a means for disengaging volume limiter 50 from plunger 25 to allow plunger 25 to travel further and check for blood or other factors.

FIG. 15 shows a cross sectional view of one embodiment of volume limiter 50 that includes retention member 80 for automatically engaging volume limiter 50 for limiting the amount of medication that can be drawn from vial 70, thereby limiting the amount of medication that can be administered to a patient, preventing overdosing, and ensuring that the correct dosage is administered. As shown in FIG. 15, volume limiter 50 may include top surface 51, opening 52 (which may include first surface 57 and second surface 58), bottom surface 53, first flange housing element 56 and second flange housing element 59. In one or more embodiments, second flange housing element 59 may be configured to retain at least one aspect of retention member 80 while another aspect of retention member 80 may be secured around an oppositely situated side of syringe 20 such that when second end 28 of plunger 25 is depressed through opening 52, with engagement section 26 aligned with first surface 57 of opening 52, retention member 80 may enable volume limiter 50 to automatically adjust to the contour of engagement section 26 and automatically snap against and apply force on engagement section 26 and thereby limit the distance plunger 25 may travel to the distance between first end 27 and second end 28. Such means for automatically engaging volume limiter 50 may, in one or more embodiments, allow plunger 25 to be provided to a user in a drawn position with second end 28 positioned against bottom surface 53 ready for the user to (1) insert an attached needle 30 into vial 70 with the aid of guiding element 40, (2) depress plunger 25 and force air into vial 70, and (3) let pressure force plunger 25 back and fill syringe 20 with medication from vial 70 up to the point at which second end 28 engages bottom surface 53. Such configuration may limit the amount of medication that can be drawn from vial 70 and thereby limit the amount of medication that can be administered to a patient. Such configuration may provide means for at least temporarily and resiliently securing the operable connection of volume limiter 50 with syringe 20. In one or more embodiments, volume limiter 50 may be manually repositioned, shifted or otherwise moved (such as substantially perpendicularly in relation to plunger 25) by a user in order to move first surface 57 away from engagement section 26 and disengage volume limiter 50 from plunger 25. Such configuration may be one embodiment of a means for disengaging volume limiter 50 from plunger 25 to allow plunger 25 to travel further and check for blood or other factors.

FIG. 16A shows a cross sectional view of one embodiment of volume limiter 50 configured as a pin 120 which pin 120 corresponds with a channel 125 manufactured into plunger 25. In one or more embodiments, instead of limiting the amount of medication medication delivery device 10 may be drawn into syringe 20 by use of plunger 25 with an engagement section 26, the embodiment shown in FIG. 16A uses a pin-channel combination to limit the amount of medication that can be drawn from vial 70, thereby limiting the amount of medication that can be administered to a patient, preventing overdosing, and ensuring that the correct dosage is administered. In one or more embodiments, a user may pull pin 120 out and disengage volume limiter 50 from plunger 25 to allow plunger 25 to travel further. In one or more embodiments, although not shown on FIG. 16A, the location on syringe 20 wherein pin 120 may be inserted may vary, thus providing an adjustable volume limiter 50 to allow various dosages to be administered.

FIG. 16B shows a top view of the embodiments of flange 21 and pin 120 illustrated in FIG. 16A. Although not shown in FIG. 16B, in one or more embodiments, pin 120 may be inserted through the barrel 22 and plunger 25 of syringe 20.

FIG. 17A shows a cross sectional view of one embodiment of volume limiter 50 which may include an inner collar 132 and an outer collar 134 which interact to provide means for automatically engaging volume limiter 50 for limiting the amount of medication that can be drawn from vial 70, thereby limiting the amount of medication that can be administered to a patient, preventing overdosing, and ensuring that the correct dosage is administered. In one or more embodiments, outer collar 134 may include a first end 136 and a second end 137. As shown in FIG. 17A, in one or more embodiments, outer collar 134 may assume an elongated “S” shape wherein second end 137 is operably connected to the top portion of syringe 20 and protrudes inwardly towards plunger 25. In one or more embodiments, inner collar 132 may include a flexible portion 138. In one or more embodiments, inner collar 132 may assume a profile substantially similar to the circumference of syringe 20 and slideably fit within the circumference of outer collar 134. In one or more embodiments, flexible portion 138 may slide over second end 137 and protrude into syringe 20 and engage second end 28 of engagement section 26 when plunger 25 may be drawn back in order to prevent plunger 25 from travelling back any further. Such configuration may provide means for automatically engaging volume limiter 50 for limiting the amount of medication that can be drawn from vial 70, thereby limiting the amount of medication that can be administered to a patient, preventing overdosing, and ensuring that the correct dosage is administered. In one or more embodiments, inner collar 132 may be designed to slide parallel relative to plunger 25 as a user pulls on inner collar 132. In one or more embodiments, as inner collar 132 slides away from needle 30, flexible portion 138 may be positioned above second end 137, allowing flexible portion 138 to flex toward outer collar 134 and out of the path of plunger 25, thereby providing means for disengaging volume limiter 50 from plunger 25 to allow plunger 25 to travel further.

FIG. 17B shows a perspective view of the embodiment of volume limiter 50 illustrated in FIG. 17A.

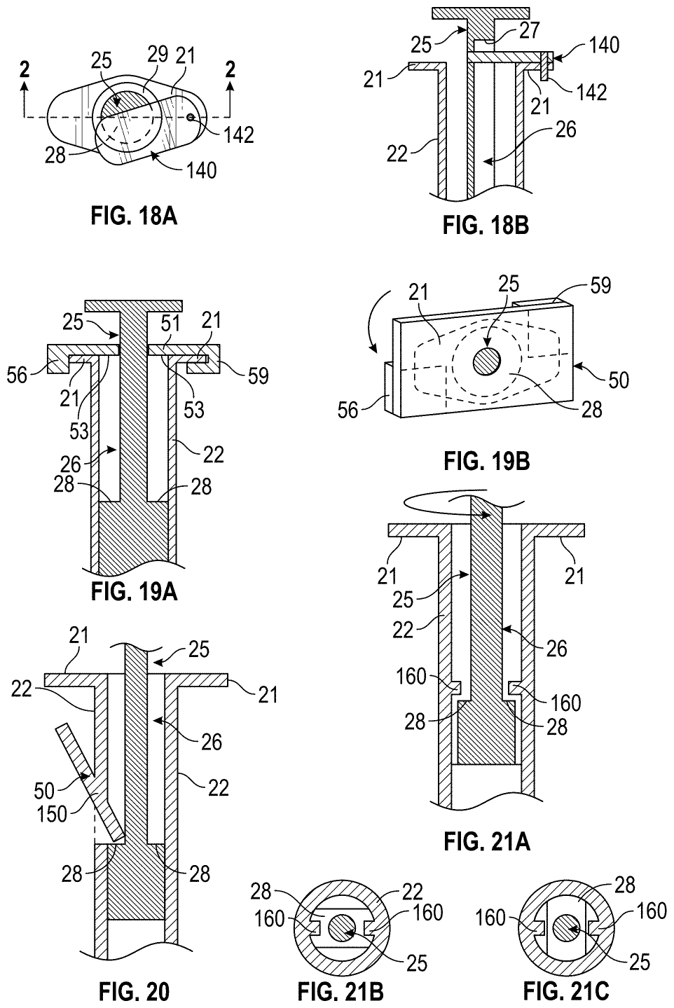

FIG. 18A shows a top view of one embodiment of volume limiter 50 including a swivelable plate 140 which engages engagement section 26 of plunger 25 to provide means for limiting the amount of medication that can be drawn from vial 70, thereby limiting the amount of medication that can be administered to a patient, preventing overdosing, and ensuring that the correct dosage is administered. In one or more embodiments, a user may pivot swivelable plate 140 around a pivot 142 in order to engage engagement section 26. In one or more embodiments, whenever a user desires to disengage volume limiter 50, the user may simply move the swivelable plate 140 and disengage it from the engagement section 26 of plunger 25. Such configuration may provide means for disengaging volume limiter 50 from plunger 25 to allow plunger 25 to travel further.

FIG. 18B shows a cross sectional view along lines 2-2 of the embodiment of volume limiter 50 including swivelable plate 140 as illustrated in FIG. 18A. In one or more embodiments, engagement section 26 may include first end 27 and second end 28 (not shown in FIG. 18B). In one or more embodiments, when engagement section 26 is aligned with swivelable plate 140, swivelable plate 140 may limit the distance plunger 25 may travel to the distance between first end 27 and second end 28, thereby limiting the amount of medication that can be drawn from vial 70, thereby limiting the amount of medication that can be administered to a patient.

FIG. 19A shows a cross sectional view of one embodiment of volume limiter 50 that may be capable of being twisted on and off of syringe 20 and of engaging and disengaging volume limiter 50 from plunger 25 to prevent and allow plunger 25 to travel. As shown in FIG. 19A, volume limiter 50 may include top surface 51, opening 52 (which may include first surface 57 and second surface 58), bottom surface 53, first flange housing element 56 and second flange housing element 59. In one or more embodiments, first flange housing element 56 and second flange housing element 59 may each engage portions of opposing ends of flange 21. To engage first flange housing element 56 and second flange housing element 59, in one or more embodiments, a user may simply twist volume limiter 50 on flange 21 in one direction. To disengage first flange housing element 56 and second flange housing element 59, in one or more embodiments, a user may simply twist volume limiter 50 off flange 21 in an opposing direction. In one or more embodiments, when engaging flange 21, first flange housing element 56 and second flange housing element 59 may prevent plunger 25 from travelling any further when drawn back by allowing bottom surface 53 to engage second end 28.

FIG. 19B shows a top perspective view of the embodiment of volume limiter 50 illustrated in FIG. 19A. In one or more embodiments, first flange housing element 56 and second flange housing element 59 may engage flange 21 by twisting around flange 21 in a clockwise rotation (or in a counterclockwise rotation in another embodiment) and first flange housing element 56 and second flange housing element 59 may disengage flange 21 by twisting around flange 21 in the opposite rotation.

FIG. 20 shows a cross sectional view of one embodiment of syringe 20 including one embodiment of volume limiter 50 built into syringe barrel 22. As shown in FIG. 20, one aspect of syringe barrel 22 may include a flexible member 150 which may include a second end 28. In one or more embodiments, plungers 25 may engage second end 28 when plunger 25 is pulled back into drawn position and thereby limit the distance plunger 25 may be pulled back and provide means for limiting the amount of medication that can be drawn from vial 70. Such configuration may provide means for automatically engaging volume limiter 50 for limiting the amount of medication that can be drawn from vial 70, thereby limiting the amount of medication that can be administered to a patient, preventing overdosing, and ensuring that the correct dosage is administered. In one or more embodiments, flexible member 150 may resiliently bend upon manipulation. In one or more embodiments, a user may manipulate flexible member 150 such that its position limits the travel distance of plunger 25 upon interaction with second end 28 or, alternatively or additionally, does not limit the travel distance of plunger 25. Such configuration may provide means for disengaging volume limiter 50 from plunger to allow plunger 25 to travel further. In one or more embodiments, flexible member 150 may be provided in a position set to limit the travel distance of plunger 25 upon interaction with second end 28 or in a non-limiting position.

FIG. 21A shows a cross sectional view of one embodiment of syringe 20 including one embodiment of volume limiter 50 built into syringe barrel 22, alternative to the configuration shown in FIG. 20. As shown in FIG. 21A, syringe barrel 22 may include volume limiter 50 built into syringe barrel 22 as protrusions 160 which limit the distance plunger 25 may travel, thereby limiting the amount of medication that can be drawn from vial 70, thereby limiting the amount of medication that can be administered to a patient, preventing overdosing, and ensuring that the correct dosage is administered. In one or more embodiments, protrusions 160 may engage second end 28 as plunger 25 is drawn back and prevent plunger 25 from being able to be drawn back further. In one or more embodiments, plunger 25 may be cut such that a user may rotate plunger 25 to position second end 28 so that second end 28 clears protrusions 160, thereby allowing plunger 25 to be drawn back further than when protrusions 160 engage second end 28. Such configuration may provide means for disengaging volume limiter 50 from plunger 25 or some other aspect of medication delivery device 10 to allow plunger 25 to travel further.

FIG. 21B shows a horizontal cross sectional view at syringe barrel's 22 protrusions 160 of the embodiments of syringe 20 illustrated in FIG. 21A, wherein volume limiter 50 is engaging plunger 25 via protrusions 160.

FIG. 21C shows a horizontal cross sectional view at syringe barrel's 22 protrusions 160 of the embodiments of syringe 20 illustrated in FIG. 21A, wherein plunger 25 has been rotated to disengage plunger 25 from protrusions 160.

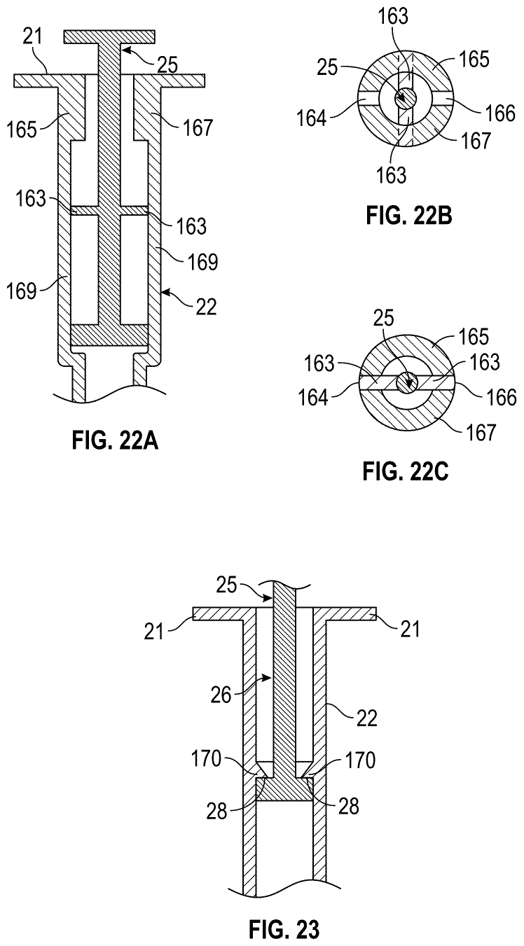

FIG. 22A shows a cross sectional view of one embodiment of plunger 25 including one embodiment of volume limiter 50 built into plunger 25 as protrusions 163. Unlike the embodiment of disclosure illustrated in FIG. 21A where volume limiter's 50 protrusions 160 may be built into syringe barrel 22, the embodiment illustrated in FIG. 22A shows protrusions 163 built into plunger 25. In one or more embodiments, syringe barrel 22 may include a first wall section 165, a second wall section 167 and a third wall section 169. In one or more embodiments, first wall section 165 and second wall section 167 may be located proximal to flange 21 and may be thicker than third wall section 169, which third wall section 169 may be distal to flange 21. In one or more embodiments, first wall section 165 and second wall section 167 may be configured to engage protrusions 163 and limit the distance plunger 25 may travel back. Such configuration may be a means for limiting the amount of medication that can be drawn from vial 70, thereby limiting the amount of medication that can be administered to a patient, preventing overdosing, and ensuring that the correct dosage is administered.

FIG. 22B shows a horizontal cross sectional view at first wall section 165 and second wall section 167 of the embodiment of syringe 20 illustrated in FIG. 22A, wherein syringe barrel 22 is engaging protrusions 163. In one or more embodiments, syringe barrel 22 may include a first channel 164 and a second channel 166 wherein protrusions 163 may pass through in order to clear first wall section 165 and second wall section 167. In one or more embodiments, as shown in FIG. 22B, first channel 164 and second channel 166 may be located on opposite sides of syringe barrel 22. In one or more embodiments, as shown in FIG. 22B, first channel 164 may be situated between first wall section 165 and second wall section 167 on one side of syringe barrel 22 and second channel 166 may be situated between first wall section 165 and second wall section 167 on an alternate side of syringe barrel 22.

FIG. 22C shows a horizontal cross sectional view at first wall section 165 and second wall section 167 of the embodiment of syringe 20 illustrated in FIG. 22A, wherein plunger 25 has been rotated to disengage protrusions 163 from syringe barrel 22. In one or more embodiments, a user may rotate plunger 25 such that protrusions 163 clear first wall section 165 and second wall section 167 and such that one protrusion 163 may travel along first channel 164 and another protrusion 163 may travel along second channel 166, allowing plunger 25 to travel further back in comparison to when protrusions 163 may be engaged by first wall section 165 and second wall section 167. Such configuration may provide means for disengaging protrusions 163 from syringe barrel 22 to allow plunger 25 to travel further.

FIG. 23 shows a cross sectional view of one embodiment of syringe 20 including one embodiment of volume limiter 50 built into syringe barrel 22, which configuration may be a permanent volume limiter 50 configuration. As shown in FIG. 23, in one or more embodiments, syringe barrel 22 may include volume limiter 50 built into syringe barrel 22 as protrusion 170 for limiting the distance plunger 25 may travel by engaging second end 28, thereby limiting the amount of medication that can be drawn from vial 70, thereby limiting the amount of medication that can be administered to a patient, preventing overdosing, and ensuring that the correct dosage is administered.

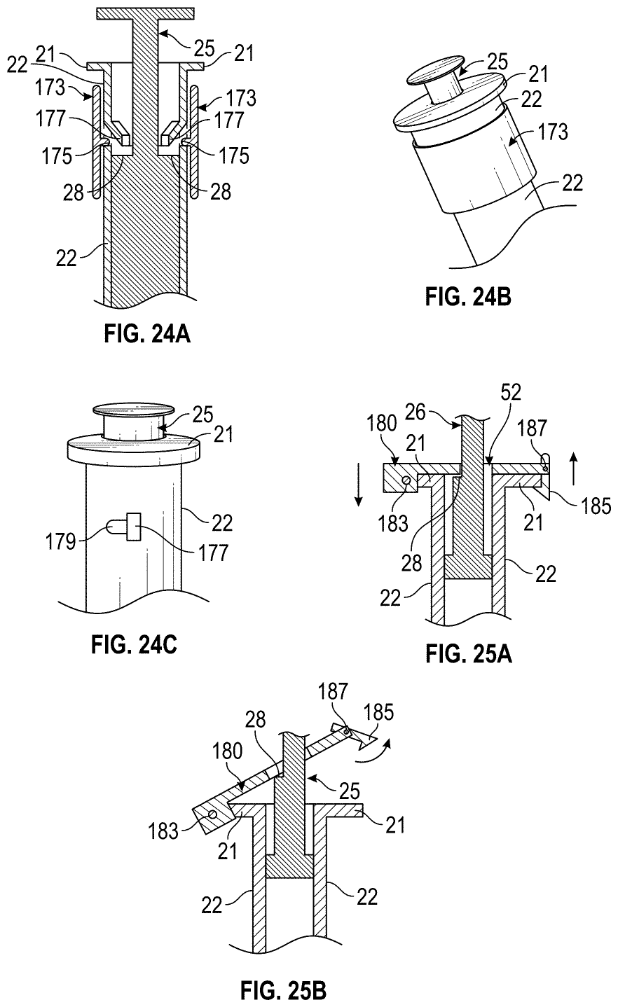

FIG. 24A shows a cross sectional view of one embodiment of volume limiter 50 configured as a single collar 173 including at least one protrusion 175. In one or more embodiments, the at least one protrusion 175 may protrude from single collar 173 and may engage at least one flexible section 177 of syringe barrel 22. In one or more embodiments, a user may twist collar 173 around syringe barrel 22 such that the at least one protrusion 175 may engage the at least one flexible section 177 of syringe barrel 22 and engage second end 28 and prevent plunger 25 from traveling further back, thereby limiting the amount of medication that can be drawn from vial 70. Such configuration may provide means for automatically engaging volume limiter 50 for limiting the amount of medication that can be drawn from vial 70, thereby limiting the amount of medication that can be administered to a patient, preventing overdosing, and ensuring that the correct dosage is administered.

FIG. 24B shows a perspective view of the embodiment of volume limiter 50 illustrated in FIG. 24A.

FIG. 24C shows a perspective view of the embodiment of syringe barrel 22 illustrated in FIG. 24A including at least one flexible section 177 and a pocket 179 wherein at least one protrusion 175 may reside when disengaged from the at least one flexible section 177. In one or more embodiments, when the at least one flexible section 177 has disengaged plunger 25, plunger 25 may travel further back as compared to when the at least one flexible section 177 engages it. Such configuration may provide means for disengaging volume limiter 50 from plunger 25 or some other aspect of medication delivery device 10 to allow plunger 25 to travel further.

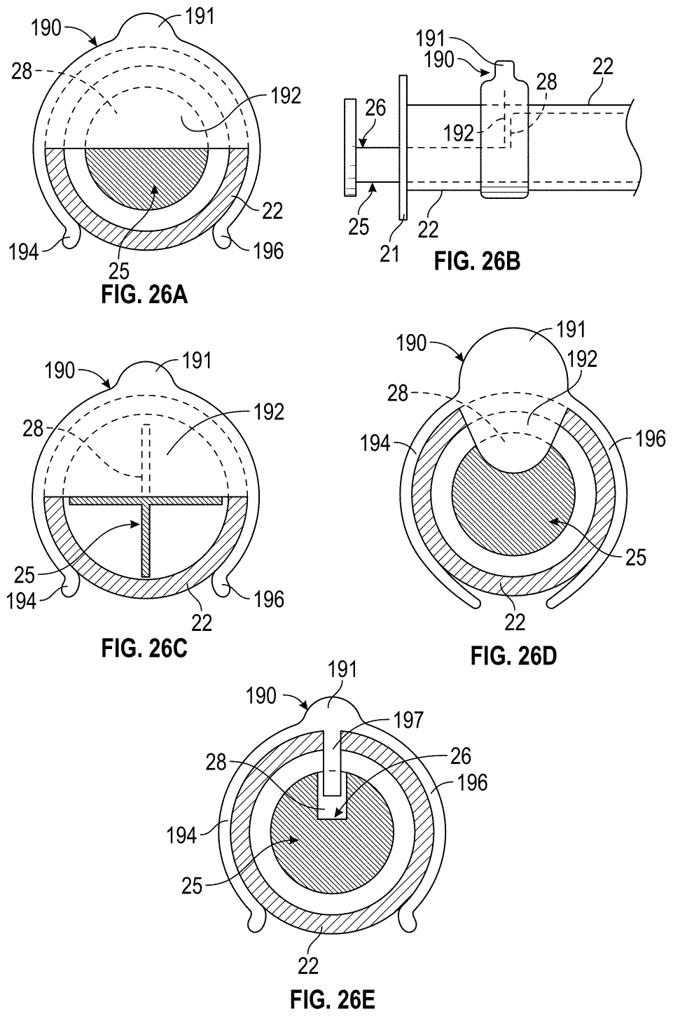

FIG. 25A shows a cross sectional view of one embodiment of volume limiter 50 configured as an articulable door 180 which may pivot about pivot 183. In one or more embodiments, articulable door 180 may engage second end 28 and prevent plunger 25 from traveling further back, thereby limiting the amount of medication that can be drawn from vial 70, thereby limiting the amount of medication that can be administered to a patient, preventing overdosing, and ensuring that the correct dosage is administered. As shown in FIG. 25A, articulable door 180 may include a hook 185 which may pivot about pivot 187. In one or more embodiments, hook 185 may engage flange 21, and when so engaged, hook 185 may prevent articulable door 180 from articulating and may aid in securing articulable door 180 to flange 21. In one or more embodiments, pivot 183 may be operably configured to flange 21. In one or more embodiments, pivot 187 may be operably configured to articulable door 180.

FIG. 25B shows a cross sectional view of the embodiment of volume limiter 50 configured as the embodiment of articulable door 180 illustrated in FIG. 25A, albeit articulable door 180 shown in FIG. 25B has been disengaged from flange 21. In one or more embodiments, a user may manipulate hook 185 and disengage it from flange 21, thereby allowing articulable door 180 to articulate about pivot 183, thereby allowing plunger 25 to travel further back as compared to when articulable door 180 is secured to flange 21 and unable to articulate about pivot 183. Such configuration may provide means for disengaging volume limiter 50 from plunger 25 to allow plunger 25 to travel further.

FIG. 26A shows a top view of one embodiment of volume limiter 50 configured as a detachable collar 190 and a horizontal cross sectional view of embodiments of syringe 20 and plunger 25. In one or more embodiments, detachable collar 190 may be configured to be detachably secured to syringe barrel 22. In one or more embodiments, detachable collar 190 may include a wing member 192 designed to be inserted through the wall of syringe barrel 22 (see also FIG. 26B) to engage second end 28 of plunger 25 and prevent plunger 25 from travelling further back, thereby limiting the amount of medication that can be drawn from vial 70. Such configuration may provide means for automatically engaging volume limiter 50 for limiting the amount of medication that can be drawn from vial 70, thereby limiting the amount of medication that can be administered to a patient, preventing overdosing, and ensuring that the correct dosage is administered. In one or more embodiments, detachable collar 190 may include a first arm member 194 and a second arm member 196, at least a portion of which may be situated to engage syringe barrel 22 and aid in retaining the operable connection of detachable collar 190 to syringe barrel 22. In one or more embodiments, detachable collar 190 may include a handle 191 to aid a user in grasping and detaching detachable collar 190 from syringe barrel 22, thereby allowing plunger 25 to travel past wing member 192. Such configuration may provide means for disengaging volume limiter 50 from plunger 25 or some other aspect of medication delivery device 10 to allow plunger 25 to travel further.

FIG. 26B shows a side view of the embodiment of volume limiter 50 configured as the embodiment of detachable collar 190 illustrated in FIG. 26A.

FIG. 26C shows a top view of the embodiment of volume limiter 50 configured as the embodiment of detachable collar 190 illustrated in FIG. 26A and a horizontal cross sectional view of embodiments of syringe 20 and plunger 25, albeit plunger 25 assumes a cruciform configuration.

FIG. 26D shows a top view of one embodiment of volume limiter 50 configured as a detachable collar 190 similar to the embodiment of volume limiter 50 illustrated in FIG. 26A and a horizontal cross sectional view of embodiments of syringe 20 and plunger 25, albeit wing member 192 is smaller and handle 191 is larger (as compared to the embodiment of volume limiter 50 illustrated in FIG. 26A) and albeit first arm member 194 and second arm member 196 assume an alternative design at their tips.

FIG. 26E shows a top view of one embodiment of volume limiter 50 configured as a detachable collar 190 similar to the embodiment of volume limiter 50 illustrated in FIG. 26A and a horizontal cross sectional view of embodiments of syringe 20 and plunger 25, albeit wing member 192 is replaced with a protrusion 197 designed to be inserted through the wall of syringe barrel 22 for engaging second end 28 of plunger 25 and preventing plunger 25 from travelling further back, thereby limiting the amount of medication that can be drawn from vial 70, thereby limiting the amount of medication that can be administered to a patient, preventing overdosing, and ensuring that the correct dosage is administered.

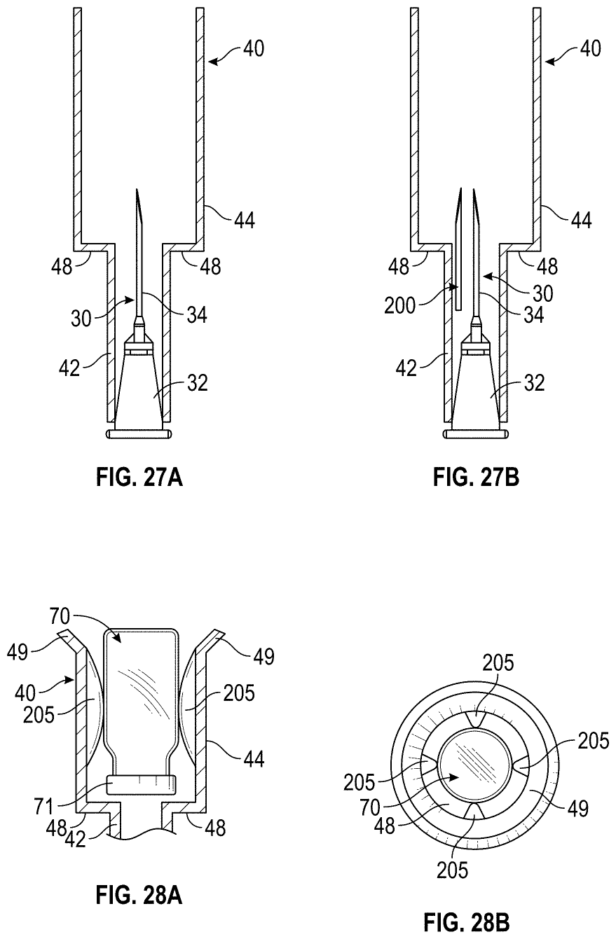

FIGS. 27A-35B show views of various embodiments of guiding element 40 that may, in one or more embodiments, be mixed and matched and substituted with other embodiments of guiding element 40 contained herein. In one or more embodiments, various embodiments of guiding element 40 illustrated in FIGS. 27A-35B may apply force symmetrically on vial 70 to center vial 70 over needle 30. In one or more embodiments, the guiding element barrels 44 illustrated in FIGS. 27A-35B may guide needle 30 into substantially the center of vial 70 and secure or otherwise engage vial 70 while a user draws medication from it. In one or more embodiments, various embodiments of guiding element 40 illustrated in FIGS. 27A-35B may apply force asymmetrically on vial 70 such that vial 70 is offset and positioned against a portion of the inner wall of guiding element barrel 44. In one or more embodiments, the guiding element shafts 42 illustrated in FIGS. 27A-35B may house and protect needle 30 and other devices, and may prevent needle sticks. In one or more embodiments, once the medication has been extracted, the guiding elements 40 illustrated in FIGS. 27A-35B may be removed from medication delivery device 10 to expose needle 30 so that the medication may be administered to the patient.

FIG. 27A shows a side view of one embodiment of needle 30 and a cross sectional view of one embodiment of guiding element 40 which may include a transition section 48 between guiding element shaft 42 and guiding element barrel 44. In one or more embodiments, transition section 48 may form a substantially right angle designed to engage cap 71 of vial 70 and control the depth needle 30 may access vial 70. In one or more embodiments, transition section 48 may aid in stabilizing the position of vial 70 while it is secured by guiding element barrel 44. In one or more embodiments, the embodiment of guiding element 40 illustrated in FIG. 27A may be used in conjunction with a medication delivery device 10 capable of automatically engaging an embodiment of volume limiter 50 for limiting the amount of medication that can be drawn from vial 70.

FIG. 27B shows a side view of the embodiment of needle 30 and a cross sectional view of the embodiment of guiding element 40 illustrated in FIG. 27A, albeit guiding element 40 includes a venting needle 200 which, in one or more embodiments, may puncture cap 71 of vial 70 and allow air to ingress into vial 70 as medication may be withdrawn from vial 70 in order to prevent formation of a vacuum that may inhibit its flow into syringe 20. In one or more embodiments, venting needle 200 may eliminate or reduce the need to force air into vial 70 to prevent a vacuum. In one or more embodiments, the embodiment of guiding element 40 illustrated in FIG. 27B may be used in conjunction with a medication delivery device 10 wherein the volume limiter 50 is manually engaged for limiting the amount of medication that can be drawn from vial 70.

FIGS. 28A-33 show various embodiments of guiding element 40 designed with a means to accommodate different sized vials 70. In one or more embodiments, guiding element 40 may be configured with inserts, stepped walls, a spring-loaded interior sleeve and/or expanding walls or other materials to accommodate the full range of vial sizes. In one or more embodiments, the embodiments of guiding element 40 illustrated in FIGS. 28A-33 may be used in conjunction with medication delivery devices 10 capable of automatically engaging embodiments of volume limiter 50 for limiting the amount of medication that can be drawn from vials 70. In one or more embodiments, the embodiments of guiding element 40 illustrated in FIGS. 28A-33 may include a transition section 48 between its guiding element shaft 42 and guiding element barrel 44 which may form a substantially right angle designed to engage the caps 71 of vials 70 and control the depth needles 30 may access vials 70 and aid in stabilizing vials 70 while they are secured in the guiding element barrel 44. In one or more embodiments, the embodiments of guiding element barrel 44 illustrated in FIGS. 28A-33 may include angled edges 49 for guiding vial 70 into guiding element barrel 44.

FIG. 28A shows a cross sectional view of one embodiment of guiding element 40 including at least two bow members 205 for engaging and securing vial 70 when vial 70 is inserted into guiding element barrel 44. In one or more embodiments, the at least two bow members 205 may flex towards guiding element barrel 44 or otherwise move as vial 70 is inserted into guiding element barrel 44. In one or more embodiments, the at least two bow members 205 may flex towards guiding element barrel 44 at increasing angles as larger vials 70 are inserted into guiding element barrel 44. In one or more embodiments, the at least two bow members 205 may apply such force to vial 70 as necessary to secure vial 70 in guiding element barrel 44. Although not shown in FIG. 28A, in one or more embodiments, vial 70 may be removed from guiding element barrel 44 as desired by a user.

FIG. 28B shows a bottom view of the embodiment of guiding element 40 illustrated in FIG. 28A configured with four of the at least two bow members 205. Although not shown in FIG. 28A or 28B, in one or more embodiments, the at least two bow members 205 may assume a “Y” formation which provide three points of contact with vial 70. Although not shown in FIG. 28A or 28B, in one or more embodiments, guiding element 40 may include a single bow member 205 for engaging and securing vial 70 when vial 70 is inserted into guiding element barrel 44. In such a configuration, the single bow member 205 may engage vial 70 such that vial 70 is offset.

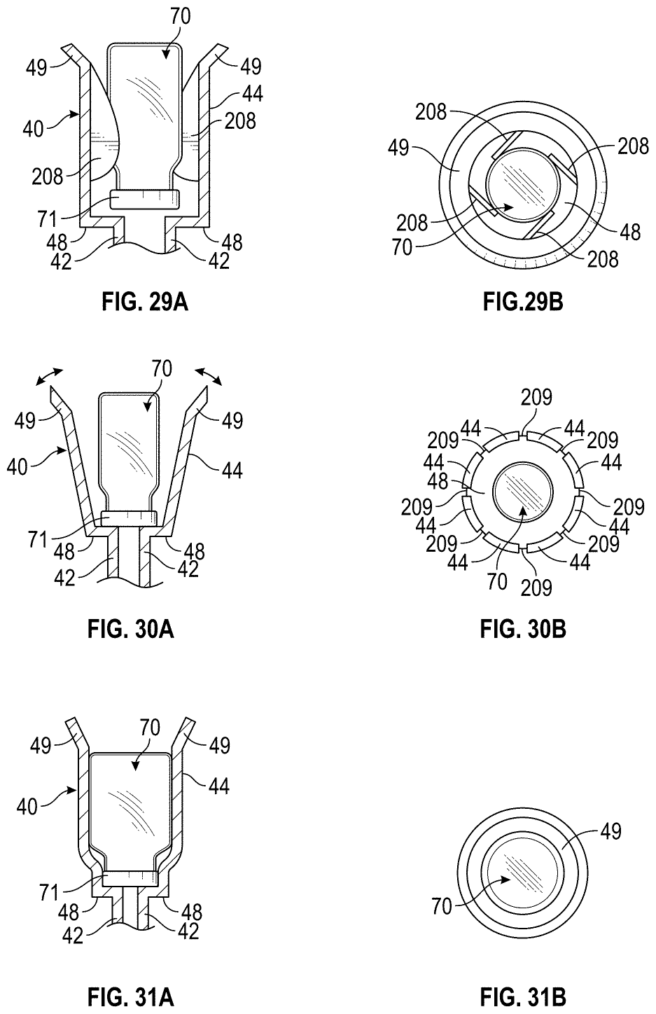

FIG. 29A shows a cross sectional view of one embodiment of guiding element 40 including at least three fins 208 (only two fins 208 are shown in FIG. 29A) for engaging and securing vial 70 when vial 70 is inserted into guiding element barrel 44. As shown in FIG. 29A, the at least three fins 208 may flex, pivot or articulate towards guiding element barrel 44 or otherwise move as vial 70 is inserted into guiding element barrel 44. In one or more embodiments, the at least three fins 208 may flex towards guiding element barrel 44 at decreasing angles as larger vials 70 are inserted into guiding element barrel 44. In one or more embodiments, the at least three fins 208 may apply such force to vial 70 as necessary to secure vial 70 in guiding element barrel 44. Although not shown in FIG. 29A, in one or more embodiments, vial 70 may be removed from guiding element barrel 44 as desired by a user.

FIG. 29B shows a bottom view of the embodiment of guiding element 40 illustrated in FIG. 29A.

FIG. 30A shows a cross sectional view of one embodiment of guiding element 40 including guiding element barrel 44 with independently movable sections of its wall. Although not shown in FIG. 30A, the independently movable sections of the wall of guiding element barrel 44 may be designed to automatically adjust to the contour of vial 70 and engage and secure vial 70 when vial 70 is inserted into guiding element barrel 44. As shown in FIG. 30A, a space 209 may be located between each independently movable section of the wall of said guiding element barrel 44.

FIG. 30B shows a bottom view of the embodiment of guiding element 40 illustrated in FIG. 30A.

FIG. 31A shows a cross sectional view of one embodiment of guiding element 40 including guiding element barrel 44, at least a portion of which may be designed with flexible or semi-flexible material that may automatically adjust to the contour of vial 70 and engage and secure vial 70 when vial 70 is inserted into guiding element barrel 44.

FIG. 31B shows a bottom view of the embodiment of guiding element 40 illustrated in FIG. 31A.

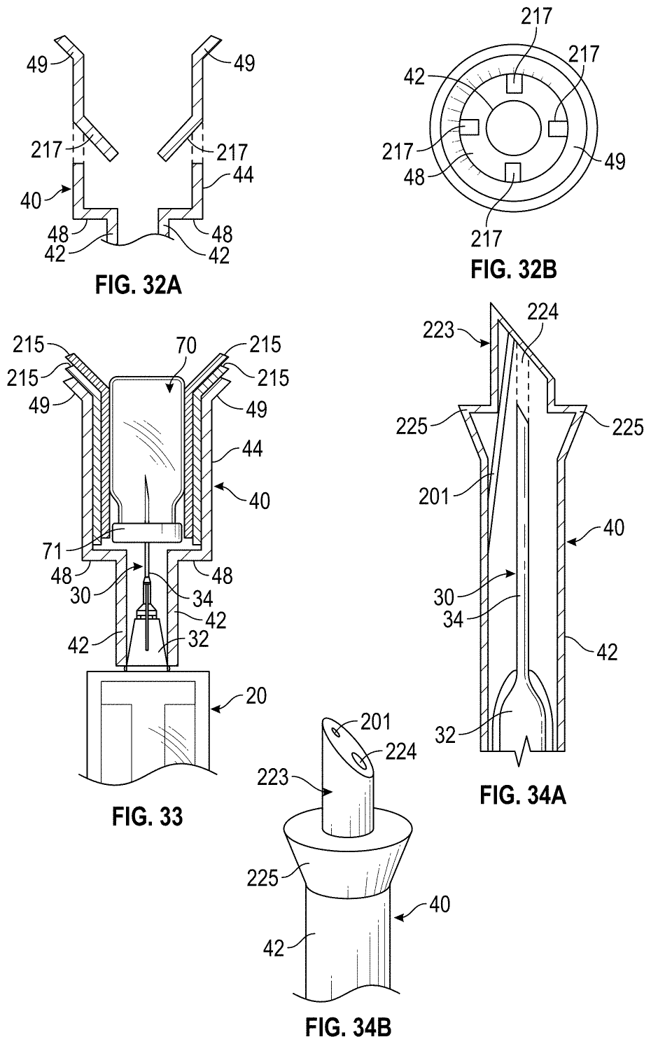

FIG. 32A shows a cross sectional view of one embodiment of guiding element 40 including at least one flexible member 217 for engaging and securing vial 70 when vial 70 is inserted into guiding element barrel 44. In one or more embodiments, the at least one flexible member 217 may flex towards guiding element barrel 44 or otherwise move as vial 70 is inserted into guiding element barrel 44 and then once cap 71 passes the end of the at least one flexible member 217, said at least one flexible member 217 may apply such force to vial 70 as necessary to secure vial 70 in guiding element barrel 44. In one or more embodiments, the embodiment of guiding element barrel 44 as illustrated in FIG. 32A may be designed to permanently retain vial 70 once it is placed in guiding element barrel 44.

FIG. 32B shows a bottom view of the embodiment of guiding element 40 illustrated in FIG. 32A.

FIG. 33 shows a cross sectional view of one embodiment of guiding element 40 with at least one insert 215 placed between guiding element barrel 44 and vial 70 in order to aid in the engagement, securement and stabilization of vial 70 when vial 70 is inserted into guiding element barrel 44. In one or more embodiments, the at least one insert 215 may be made from various materials, including rubber, plastic or other materials.

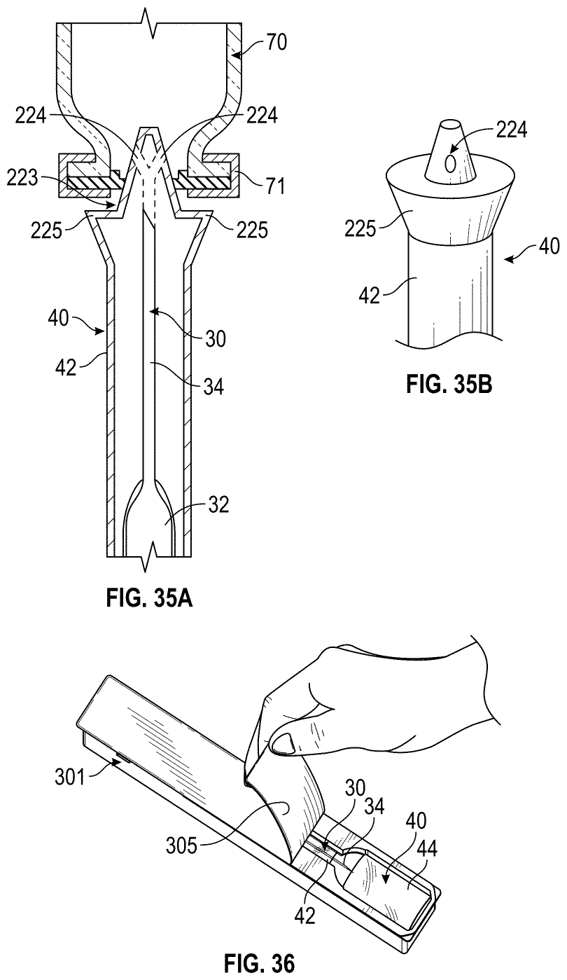

FIGS. 34A-35B show various embodiments of guiding element 40 assuming a cannula like configuration.

FIG. 34A shows a cross sectional view of one embodiment of guiding element 40 including guiding element shaft 42 configured like a semi-sharp cannula including a neck 223 designed to be sharp enough to puncture the rubber septum of vial 70 but not sharp enough to penetrate skin and a duct 224 where medication may flow from vial 70 to needle 30. Such configuration may provide a safe compact unit which may not require extensive fine motor control. As shown in FIG. 34A, in one or more embodiments, the cannula-like designed guiding element shaft 42 may house or enclose needle 30. Such configuration may prevent needle sticks and avoid having to adapt the disclosure to accommodate different sized vials 70 as one size cannula-like designed guiding element shaft 42 may fit various sized vials 70. In one or more embodiments, after the cannula-like designed guiding element shaft 42 is inserted into vial 70, medication from vial 70 may flow through duct 224, into and through needle 30 and into syringe 20. In one or more embodiments, after the cannula-like configured guiding element shaft 42 may be used to draw medication from vial 70, a user may remove the cannula-like configured guiding element shaft 42, thereby exposing the pre-assembled needle 30 and perform the injection. Such configuration may avoid the disadvantages of using available cannula systems which required switching out the cannula after it is used to draw medication and then replacing it with a needle for injecting a patient. As indicated, in one or more embodiments, the cannula-like designed guiding element shaft 42 may only require removing the guiding element shaft 42 to expose an already attached needle 30, thus, in comparison to available cannulas, the disclosure reduces both the time and the number steps it takes for the procedure and the chance of dropping or otherwise contaminating or damaging needle 30 or others. Although not shown in FIG. 34A, the cannula-like designed guiding element shaft 42 may include safety shield 60 for covering needle 30 after use. In one or more embodiments, the cannula-like designed guiding element shaft 42 may only include a single needle 30. In one or more embodiments, the cannula-like designed guiding element shaft 42 may house more than one needle, such as venting needle 200 and needle 30. As shown in FIG. 34A, in one or more embodiments, cannula-like designed guiding element shaft 42 may include a venting channel 201 which may allow air to ingress into vial 70 as medication may be withdrawn from vial 70 in order to prevent the formation of a vacuum that may inhibit its flow into syringe 20. In one or more embodiments, the cannula-like designed guiding element shaft 42 may be configured to engage cap 71 or another aspect of vial 70 in such a way so as to prevent guiding element 40 from traveling into vial 70 beyond a certain point. For example, as shown in FIG. 34A, end 225 of the cannula-like designed guiding element shaft 42 may be flared to prevent over-travel into vial 70.

FIG. 34B shows a perspective view of the embodiment of guiding element 40 illustrated in FIG. 34A.

FIG. 35A shows a cross sectional view of one embodiment of guiding element 40 with neck 223 assuming a cone configuration and duct 224 assuming a “Y” configuration. As shown in FIG. 35A, in one or more embodiments, these cone and duct 224 configurations allow two pathways for medication to flow from vial 70 into needle 30 and may only need a single needle 30 to be present (i.e. no venting needle 200 is illustrated in FIG. 35A).

FIG. 35B shows a perspective view of the embodiment of guiding element 40 illustrated in FIG. 35A. In one or more embodiments, the embodiment of guiding element 40 illustrated and described in relation to FIGS. 35A and 35B, may be substantially similar to the embodiment of guiding element 40 illustrated and described in relation to FIGS. 34A and 34B, except for the features mentioned above and illustrated in FIGS. 35A and 35B.

FIG. 36 shows a perspective view of one embodiment of medication delivery device 10 in a blister package 301 (or simply, package 301) with a blister lid 305 being peeled open. Although not shown in FIG. 36, in one or more embodiments, vial 70 of medication may be sold or otherwise provided in package 301 with medication delivery device 10. In one or more embodiments, vial 70 of medication may be sold or otherwise provided separate from package 301, allowing users to replace the medication without being required to also replace medication delivery device 10. In one or more embodiments, the expiration date of medication delivery device 10 may not be dependent upon the expiration date of the medication that is to be administered to patients. In one or more embodiments, a patient may be able to replace outdated medication without having to replace medication delivery device 10, thereby allowing users to keep quality medication on hand without having to incur additional expense of replacing medication delivery device 10.

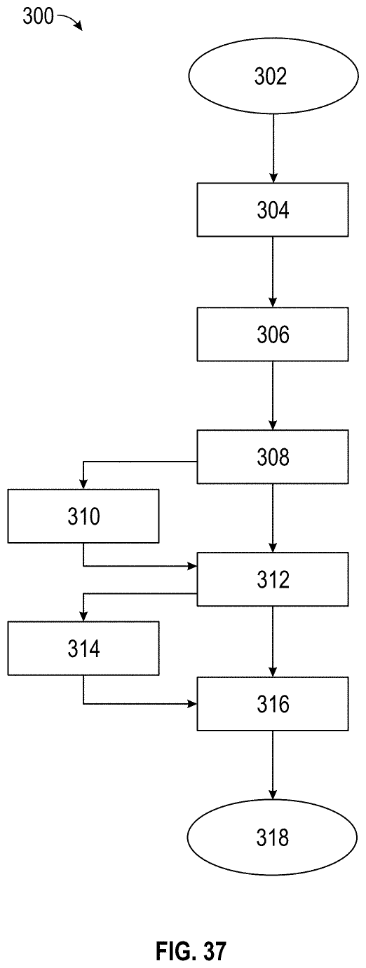

FIG. 37 shows a flow diagram that depicts one embodiment of a method 300 for using medication delivery device 10. The method 300 for using medication delivery device 10 as illustrated in flow diagram FIG. 37 may be customized, flexible and adapted to various circumstances and situations (including various embodiments of medication delivery device 10, volume limiter 50, guiding element 40, needles 30, syringe 20, and plunger 25, such as those shown and described in relation to FIGS. 1-183). Method 300 may be used to administer medication or other substances to patients. In step 302, a user enters the process. In one or more embodiments, the user may open package 301 and remove an embodiment of medication delivery device 10 that has been preassembled with embodiments of volume limiter 50, guiding element 40, and needle 30 operably connected to syringe 20. Alternatively, in one or more embodiments, a user may assemble medication delivery device 10 by operably connecting an embodiment of volume limiter 50 and/or an embodiment of guiding element 40 and/or an embodiment of needle 30 to syringe 20. A user may hold an assembled or preassembled medication delivery device 10 in an upright position such that guiding element 40 is pointed up. In one or more embodiments, the user may remove a safety cap from needle 30 if present. In step 304, a user may insert vial 70 into guiding element barrel 44 of guiding element 40 such that needle 30 accesses vial 70 below the medication level. In step 306, a user may draw back on plunger 25 to fill syringe 20 with medication until volume limiter 50 engages the plunger 25 and prevents it from traveling further back (such as when second end 28 of engagement section 26 engages bottom surface 53 of volume limiter 50). In one or more embodiments, plunger 25 may be sold or otherwise provided already depressed into the syringe barrel 22 such that a user may draw back on it as described in step 306. In one or more embodiments, the user may verify dose. In step 308, a user may remove guiding element 40 and vial 70 from needle 30 to expose needle 30. In one or more embodiments, a user may need to expel excess air from syringe 20 after removing vial 70. In step 310, a user may optionally disengage engagement section 26 of plunger 25 from volume limiter 50, such as by manually rotating plunger 25 such that engagement section 26 is no longer in contact with the first surface 57 of opening 52 (or is no longer in contact with notch 88 in the case of a cruciform plunger 25) and/or such as by shifting the volume limiter 50 into an alternative position which disengages first surface 57 from the engagement section 26 of the plunger 25. In step 312, a user may insert needle 30 into a patient. In step 314, a user may optionally draw back plunger 25 to check for placement, flash or for other purposes. In step 316, a user may depress plunger 25 to inject the medication into the patient. In step 318, a user may withdraw medication delivery device 10 from the patient and end the process.

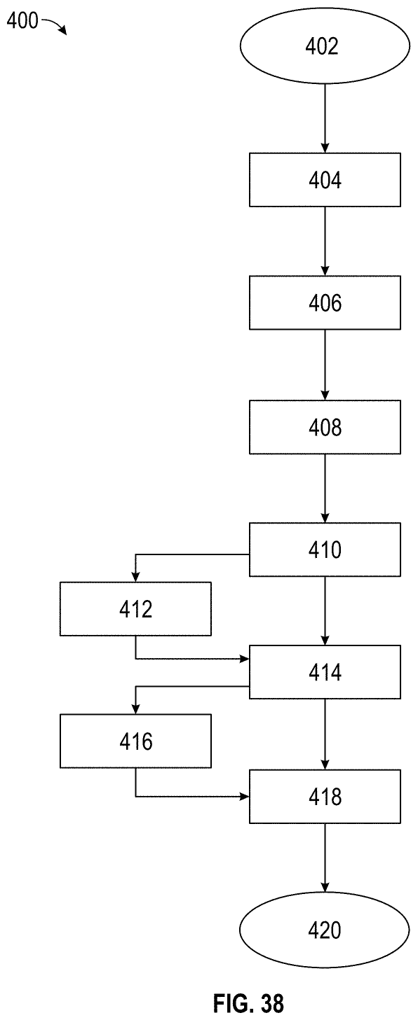

FIG. 38 shows a flow diagram that depicts one embodiment of a method 400 for using medication delivery device 10. The method 400 for using medication delivery device 10 as illustrated in flow diagram FIG. 38 may be customized, flexible and adapted to various circumstances and situations (including various embodiments of medication delivery device 10, volume limiter 50, guiding element 40, needles 30, syringe 20, and plunger 25, such as those shown and described in relation to FIGS. 1-183). Method 400 may be used to administer medication or other substances to patients. In step 402, a user enters the process. In one or more embodiments, the user may open package 301 and remove an embodiment of medication delivery device 10 that has been preassembled with embodiments of volume limiter 50, guiding element 40, and needle 30 operably connected to syringe 20. Alternatively, in one or more embodiments, a user may assemble medication delivery device 10 by operably connecting an embodiment of volume limiter 50 and/or an embodiment of guiding element 40 and/or an embodiment of needle 30 to syringe 20. A user may hold an assembled or preassembled medication delivery device 10 in an upright position such that guiding element 40 is pointed up. In one or more embodiments, the user may remove a safety cap from needle 30 if present. In step 404, a user may insert vial 70 into guiding element barrel 44 of guiding element 40 such that needle 30 accesses vial 70 below the medication level. In step 406, a user may depress plunger 25 to force air into vial 70. In one or more embodiments, the user may depress plunger 25 to engage volume limiter 50. In one or more embodiments, the user may depress plunger 25 either until the plunger stopper 29 engages the bottom of syringe barrel 22 or until volume limiter 50 prevents the further travel of plunger 25 (such as when first end 27 of engagement section 26 engages top surface 51 of volume limiter 50). In one or more embodiments, plunger 25 may be sold or otherwise provided already inserted through opening 52 of volume limiter 50 but not yet depressed into the syringe barrel 22. In one or more embodiments, although plunger 25 may come already inserted through opening 52 of volume limiter 50, volume limiter 50 may not be fully engaged until plunger 25 is depressed as described in step 406. In one or more embodiments, a pressurized vial 70 may fill syringe 20 until plunger 25 is stopped by volume limiter 50. In step 408, a user may allow plunger 25 to automatically draw back and syringe 20 to fill with the medication until volume limiter 50 prevents further travel of plunger 25 (such when second end 28 of engagement section 26 engages bottom surface 53 of volume limiter 50). In step 410, a user may remove guiding element 40 and vial 70 from needle 30 to expose needle 30. In one or more embodiments, a user may need to expel excess air from syringe 20 after removing vial 70. In one or more embodiments, the user may verify dose. In step 412, a user may optionally disengage engagement section 26 of plunger 25 from volume limiter 50, such as by manually rotating plunger 25 such that engagement section 26 is no longer in contact with the first surface 57 of opening 52 (or is no longer in contact with notch 88 in the case of a cruciform plunger 25) and/or such as by shifting the volume limiter 50 into an alternative position which disengages first surface 57 from the engagement section 26 of the plunger 25. In step 414, a user may insert needle 30 into a patient. In step 416, a user may optionally draw back plunger 25 to check for placement, flash or for other purposes. In step 418, a user may depress plunger 25 to inject the medication into the patient. In step 420, a user may withdraw medication delivery device 10 from the patient and end the process.

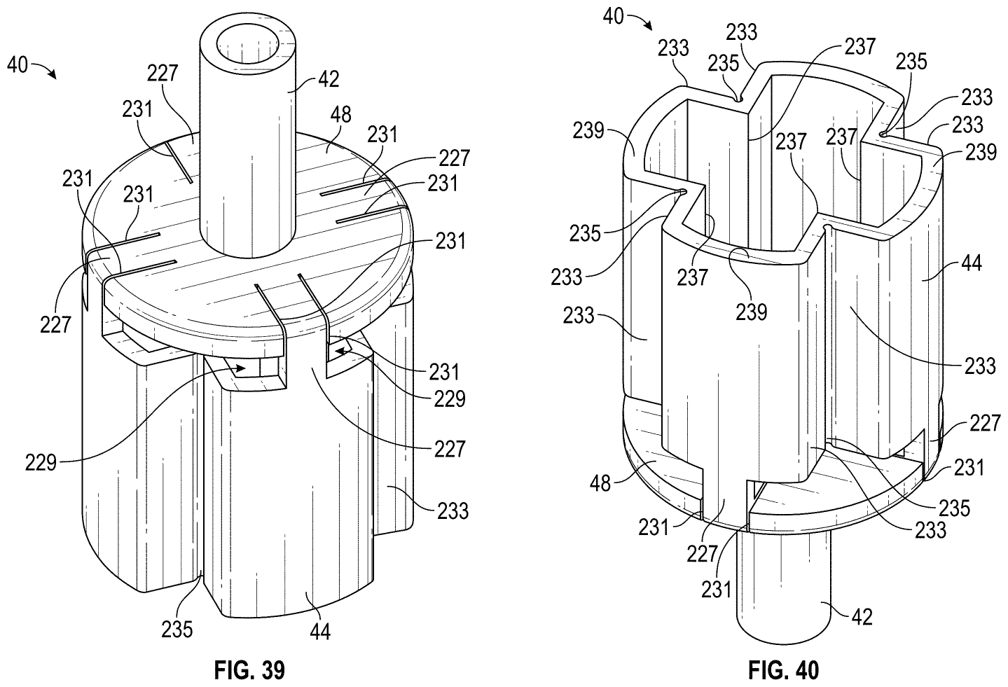

FIGS. 39-56 show various embodiments of guiding element 40 designed with means to accommodate different sized vials 70. In particular, FIGS. 39-56 show various embodiments of guiding element 40 configured for at least the purpose of providing guiding element barrels 44 that resiliently flex or expand and contract in order to house vials 70 of different sizes. In one or more embodiments, the guiding elements 40 shown and described in relation to FIGS. 39-56 may include the structures and functionalities of the other embodiments of guiding element 40 shown and described herein (which structures and functionalities of said embodiments are hereby incorporated herein and applied to the guiding elements 40 shown and described in relation to FIGS. 39-56 by reference). For example, in one or more embodiments, the guiding elements 40 illustrated in FIGS. 39-56 may be used in conjunction with medication delivery devices 10 capable of automatically engaging various embodiments of volume limiter 50 for limiting the amount of medication that can be drawn from vials 70. In one or more embodiments, the guiding elements 40 illustrated in FIGS. 39-56 may be mixed and matched and substituted with other embodiments of guiding element 40 contained herein and used in conjunction with various embodiments of volume limiter 50 as part of medication delivery devices 10. In one or more embodiments, the guiding elements 40 illustrated in FIGS. 39-56 may include a transition section 48 between its guiding element shaft 42 and guiding element barrel 44 which may form a substantially right angle designed to engage the caps 71 of vials 70 and control the depth needles 30 may access vials 70 and aid in stabilizing vials 70 while they are secured in the guiding element barrel 44. In one or more embodiments, the guiding element barrels 44 illustrated in FIGS. 39-56 may guide needle 30 into substantially the center of vial 70 and secure or otherwise engage vial 70 while a user draws medication from it. In one or more embodiments, the guiding element shafts 42 illustrated in FIGS. 39-56 may house and protect needle 30 and other devices, and may prevent needle sticks. In one or more embodiments, once the medication has been extracted, the guiding elements 40 illustrated in FIGS. 39-56 may be removed from medication delivery device 10 to expose needle 30 so that the medication may be administered to the patient.

FIG. 39 shows a top perspective view of one embodiment of guiding element 40 which may include guiding element shaft 42, guiding element barrel 44, transition section 48, support arms 227, windows 229, slits 231, and recessed portions 233 of barrel 44. In one or more embodiments, the guiding element 40 shown in FIG. 39 is configured to operably connect with the hub 32 or wings 33 of a needle 30. In one or more embodiments, said needle 30 may be configured to operably connect to a syringe tip 24 configured as a Luer-Lok syringe tip, such as shown in FIG. 1 (or said needle 30 may be configured to operably connect to a safety shield 60 that is configured to operably connect to a syringe tip 24 configured as a Luer-Lok syringe tip, such as shown in FIGS. 2 and 3). Although not shown in FIG. 39, in one or more embodiments, said needle 30 may be configured to operably connect to alternative syringe tips 24 or safety shields 60. As shown in FIG. 39, in one or more embodiments, transition section 48 may include slits 231 that run from the top surface of transition section 48 through to the bottom surface of transition section 48. In one or more embodiments, slits 231 (like spaces 209 shown in FIG. 30A-30B) enable support arms 227 to independently move or articulate outwardly or inwardly (or engage in extension or flexion movements) as vial 70 is inserted into or pulled out of barrel 44. In one or more embodiments, support arms 227 may be formed by making the slits 231 and windows 229. In one or more embodiments, support arms 227 may be operably connected (such as being integral) to both the transition section 48 and the guiding element barrel 44. In one or more embodiments, support arms 227 function similar to the independently movable sections of the wall of the embodiment of the guiding element barrel 44 shown in FIGS. 30A and 30B in that they move independently and may be designed to automatically adjust to the contour of vial 70 when vial 70 is inserted into guiding element barrel 44.

FIG. 40 shows a bottom perspective view of the embodiment of guiding element 40 illustrated in FIG. 39 including a bottom barrel edge 239. In one or more embodiments, guiding element barrel 44 may include recessed portions 233. In one or more embodiments, recessed portions 233 (like the at least two bow members 205 shown in FIG. 28A-28B) flex and engage and secure vials 70 when they are inserted into guiding element barrel 44 by applying such force to vials 70 as necessary to secure them. For example, in one or more embodiments, a pair of recessed portions 233 may be bent inward and meet to form a channel 235 on the exterior surface of barrel 44 and to form a barrel engagement surface 237 on the interior surface of barrel 44. At least one purpose of barrel engagement surfaces 237 may be to engage the side of vial 70 when it is inserted into guiding element barrel 44. In one or more embodiments, engagement surfaces 237 may be rounded, sharp or otherwise configured. In one or more embodiments, channels 235 may be rounded in order to give guiding element barrel 44 the necessary flexibility to resiliently flex or expand and contract as vial 70 is inserted or withdrawn from the guiding element 40. In one or more embodiments, channels 235 may assume a profile and configuration alternative to being rounded, such as sharp or otherwise configured. At least one purpose of channels 235 (along with slits 231, support members 227, and windows 229) may be to provide a means for allowing guiding element barrel 44 to resiliently flex or expand and contract depending on the size of the vial 70 inserted into guiding element barrel 44. In one or more embodiments, guiding element barrel 44 may include a bottom barrel edge 239 that is defined by the bottom perimeter of barrel 44. In one or more embodiments, the length of each barrel engagement surface 237 may run from the bottom barrel edge 239 to each of their respective windows 229.

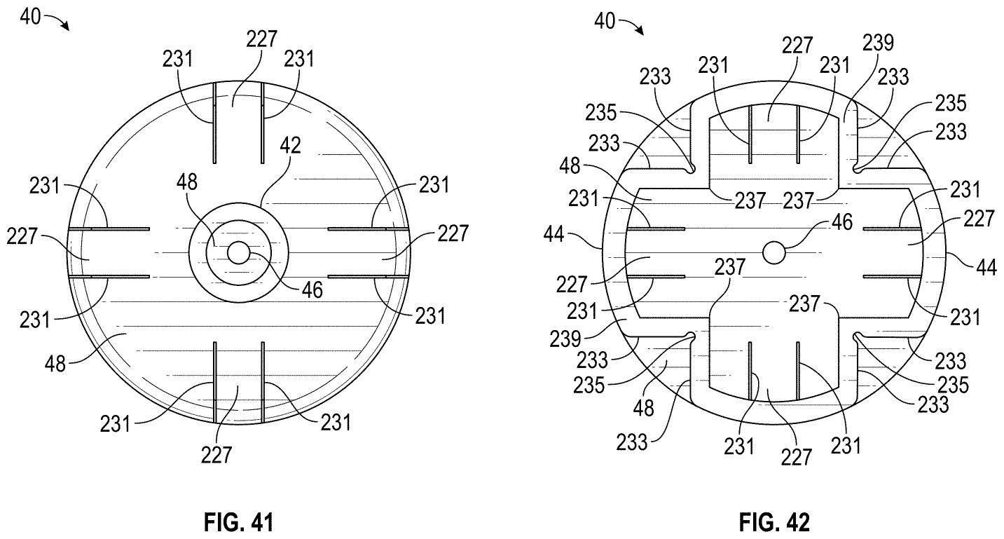

FIG. 41 shows a top view of the embodiment of guiding element 40 illustrated in FIG. 39. As shown in FIG. 41, in one or more embodiments, guiding element 40 may include eight slits 231, four support arms 227, and an opening 46 through which needle 30 is inserted.

FIG. 42 shows a bottom view of the embodiment of guiding element 40 illustrated in FIG. 39. As shown in FIG. 42, in one or more embodiments, the bottom barrel edge 239 and barrel 44 walls may resemble a “+” or plus sign formation from a bottom view. In one or more embodiments, any two recessed portions 233 that form a pair may be situated such that they are substantially 90 degrees, less than 90 degrees, or more than 90 degrees in relation to each other.

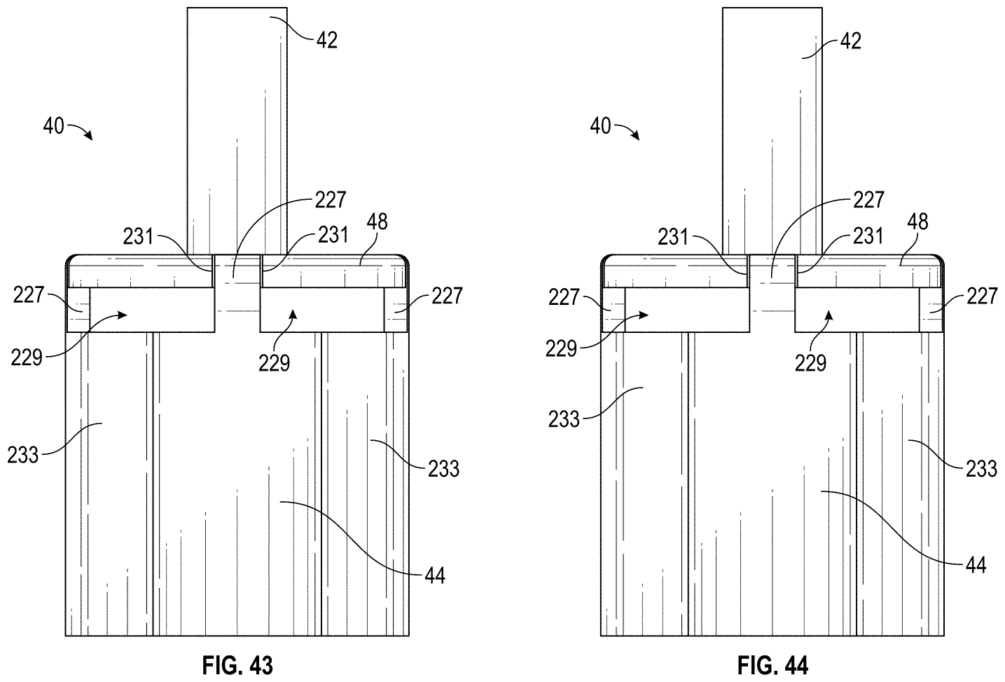

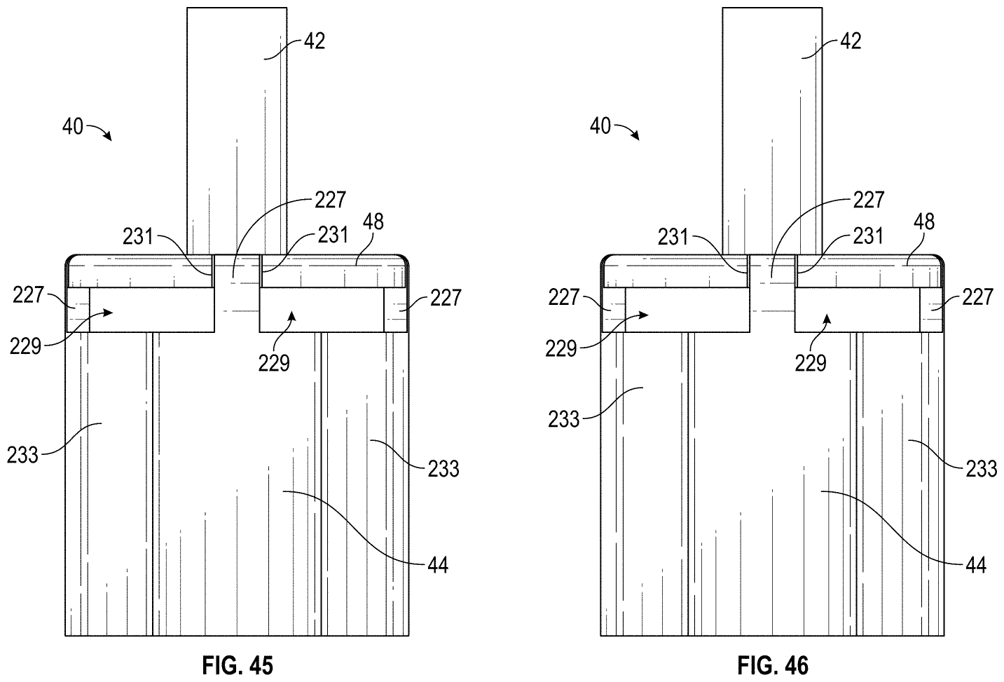

FIG. 43 shows a right side view of the embodiment of guiding element 40 illustrated in FIG. 39. FIG. 44 shows a left side view of the embodiment of guiding element 40 illustrated in FIG. 39. FIG. 45 shows a front view of the embodiment of guiding element 40 illustrated in FIG. 39. FIG. 46 shows a rear view of the embodiment of guiding element 40 illustrated in FIG. 39. As shown in FIGS. 43-46, in one or more embodiments, guiding element 40 may include four windows 229. In one or more embodiments, the bottom surface of transition section 48 and the interior surface of guiding element barrel 44 may be visible through windows 229.

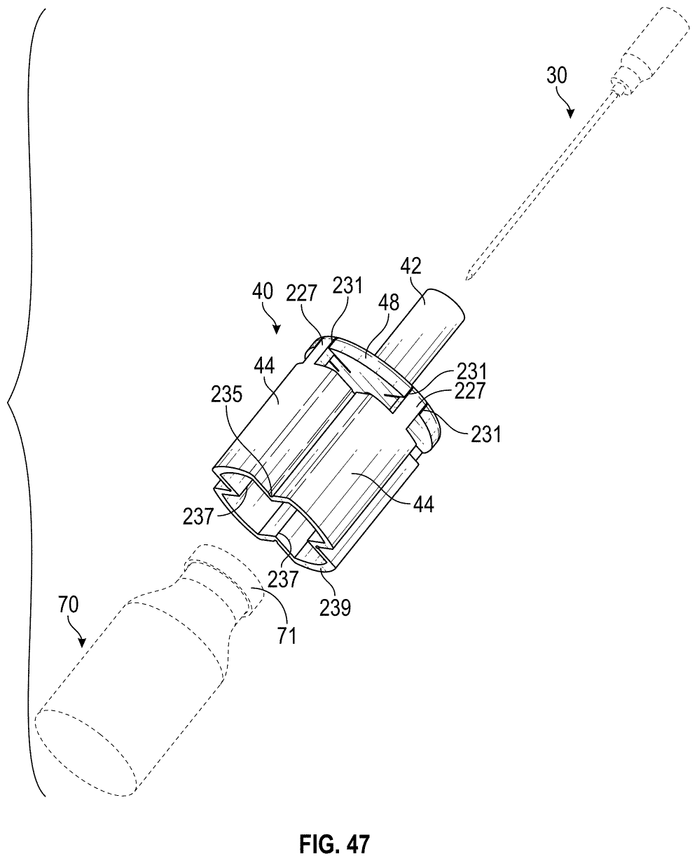

FIG. 47 shows a perspective view of the embodiment of guiding element 40 illustrated in FIG. 39 shown in an environment of use wherein guiding element 40 is configured to receive vials 70 and needles 30 of various dimensions.

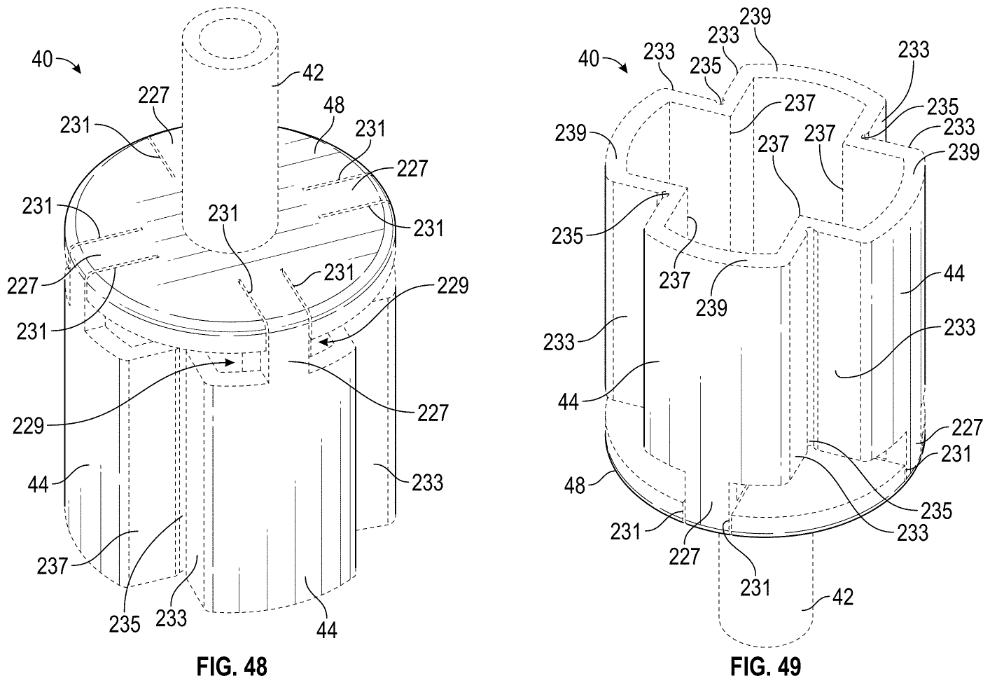

FIGS. 48-56 show that the configuration of components of the embodiment of guiding element 40 illustrated in FIGS. 39-47 may vary. For example, although FIGS. 39-47 show guiding element 40 including four support arms 227, four windows 229, eight slits 231, eight recessed portions 233, four channels 235, and four barrel engagement surfaces 237, in one or more embodiments, more or less support arm 227, windows 229, slits 231, recessed portions 233, channels 235, and barrel engagement surfaces 237 may be provided and their dimensions and locations on guiding element 40 may vary. For example, in one or more embodiments, guiding element 40 may include three support arms 227, three windows 229, six slits 231, and six recessed portions 233 bent inward meeting to form three channels 235 on the exterior surface and three barrel engagement surfaces 237 on the interior surface of barrel 44. Although FIGS. 39-47 show slits 231 cut to a certain depth and at a certain angle, in one or more embodiments, slits 231 may be made by a vertical cut or an angled cut or a multi-angled cut through the transition section 48 and, in one or more embodiments, the depth of the cuts towards the center of transition section 28 from its circumference may vary. Although FIGS. 39-47 show the bottom barrel edge 239 resembling a “+” or plus sign formation from a bottom view, in one or more embodiments, the configuration of guiding element barrel 44 may assume alternative formations, symmetrical or non-symmetrical, such as resembling a “Y” formation from a bottom view. In one or more embodiments, the possible modifications to guiding element 40 are not limited to the aspects of guiding element 40 that are dot lined in FIGS. 48-56.

FIG. 48 shows a top perspective view of one embodiment of the guiding element 40 illustrated in FIG. 39 with various components dot lined. As shown by the dotted lines in FIG. 48, in one or more embodiments, the thickness, length, location, and other dimensions and configurations of shaft 42, support arms 227, slits 231, and recessed portions 233 may vary. As shown by the dotted lines in FIG. 48, in one or more embodiments, the diameter, thickness and other dimensions and configurations of transition section 48 may vary. As shown by the dotted lines in FIG. 48, in one or more embodiments, guiding element 40 may not include support arms 227, windows 229, slits 231, or recessed portions 233 (such as shown in FIGS. 1-3, 5A-5B, and 6-8B).

FIG. 49 shows a bottom perspective view of the embodiment of guiding element 40 illustrated in FIG. 48. As shown by the dotted lines in FIG. 49, in one or more embodiments, the angle, gradualness, or sharpness at which recessed portions 233 of barrel 44 recess inward from the walls of barrel 44 may vary. For example, in one or more embodiments, the recessed portions 233 may recess inward from the walls of barrel 44 at substantially 90 degrees, less than 90 degrees, or more than 90 degrees. In one or more embodiments, any two recessed portions 233 that form a pair may be situated such that they are substantially 90 degrees, less than 90 degrees, or more than 90 degrees in relation to each other. As shown by the dotted lines in FIG. 49, in one or more embodiments, the thickness, length, diameter and other dimensions and configurations of the guiding element barrel's 44 wall may vary.

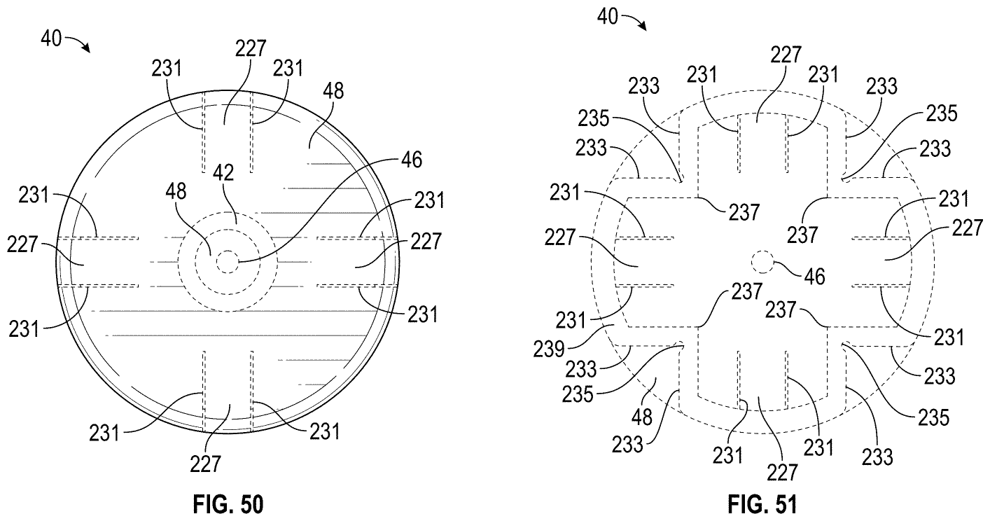

FIG. 50 shows a top view of the embodiment of guiding element 40 illustrated in FIG. 48. Although FIGS. 39-47 show shaft 42 and opening 46 at certain diameters and locations on guiding element 40, as shown by the dotted lines in FIG. 50, in one or more embodiments, the diameters and locations of shaft 42 and opening 46 may vary depending on the size of the needles 30 that guiding element 40 is designed to engage. As shown by the dotted lines in FIG. 50, in one or more embodiments, guiding element 40 may not include any slits 231.

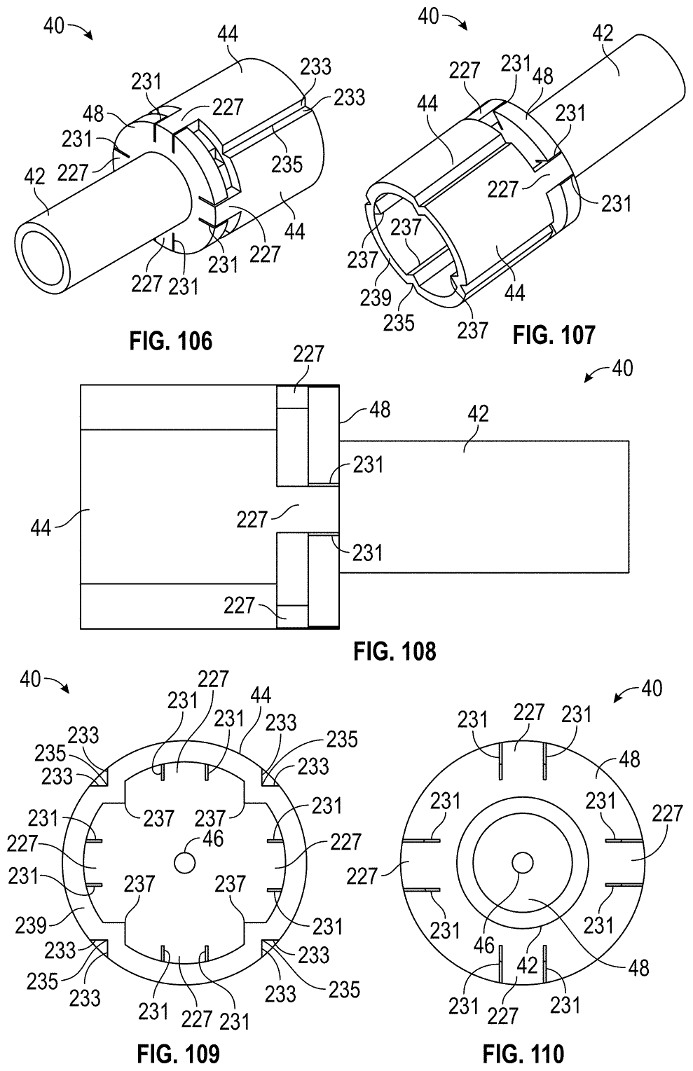

FIG. 51 shows a bottom view of the embodiment of guiding element 40 illustrated in FIG. 48. As shown by the dotted lines in FIG. 51, in one or more embodiments, the profile and other dimensions and configurations of channels 235 may vary. For example, in one or more embodiments, the channels 235 may assume a rounded profile (such as in FIGS. 39-47), a sharp profile (such as in FIGS. 106-107 and 109), an angled profile, a multi-angled profile, a multi-surfaced profile, or other profile configuration. In one or more embodiments, the angle and sharpness at which recessed portions 233 recess inward from the walls of barrel 44 and the characteristics of the channels' 235 profile, may be modified in order to provide the barrel 44 enough flexibility, rigidity, and resiliency to engage vials 70 of various dimensions. In one or more embodiments, guiding element 40 may not include channels 235 (such as shown in FIGS. 1-3, 5A-5B, and 6-8B).

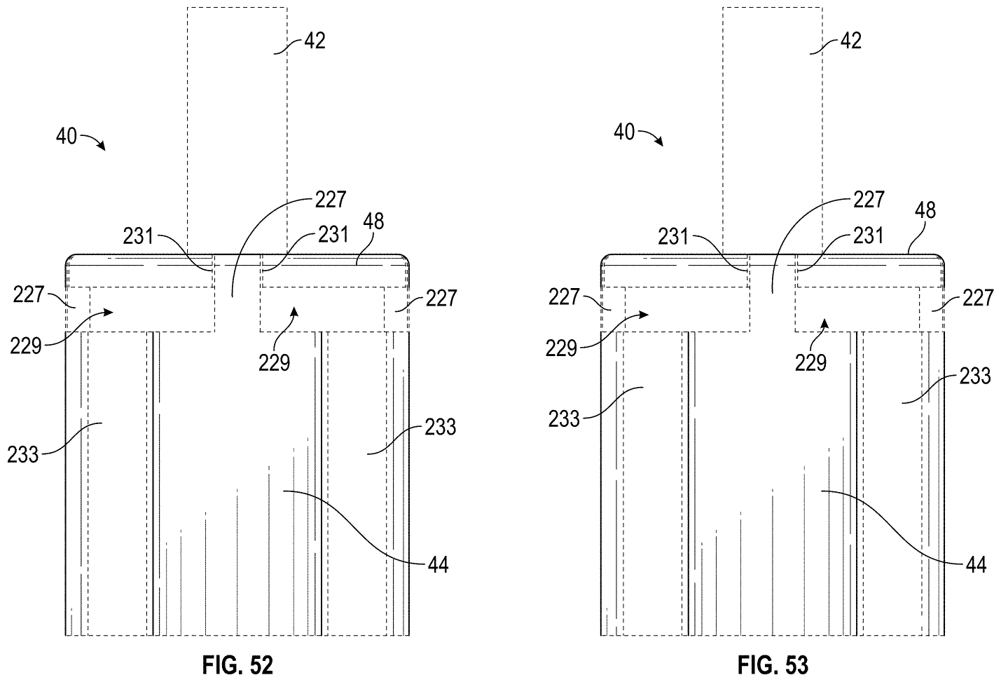

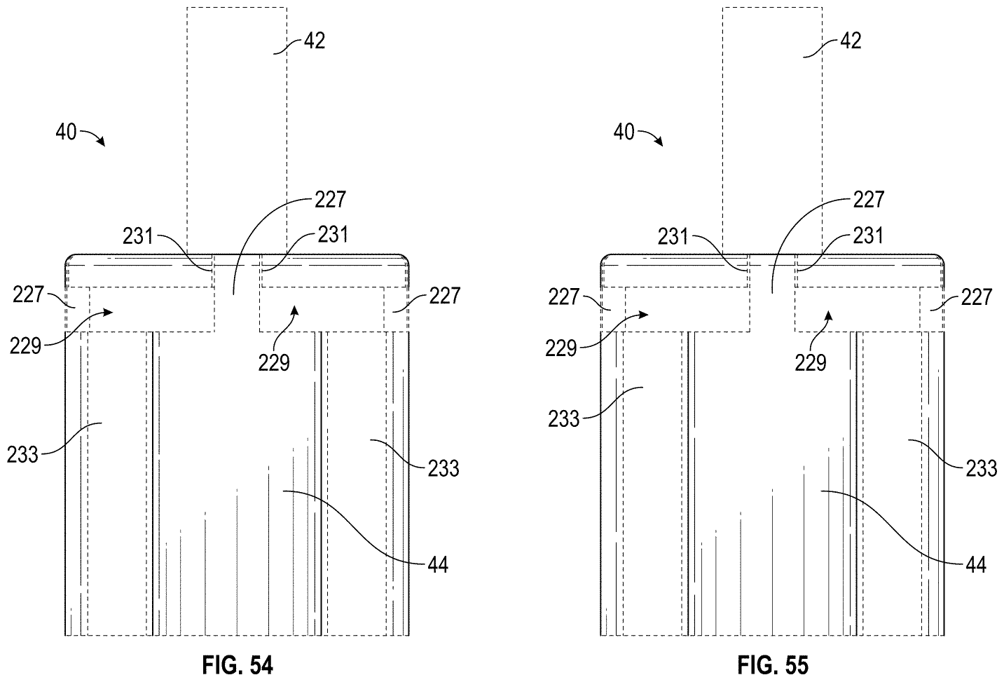

FIG. 52 shows a right side view of the embodiment of guiding element 40 illustrated in FIG. 48. FIG. 53 shows a left side view of the embodiment of guiding element 40 illustrated in FIG. 48. FIG. 54 shows a front view of the embodiment of guiding element 40 illustrated in FIG. 48. FIG. 55 shows a rear view of the embodiment of guiding element 40 illustrated in FIG. 48. As shown by the dotted lines in FIGS. 52-55, in one or more embodiments, the number, height, width and other dimensions and configurations of windows 229, slits 231, and support arms 227 may vary.

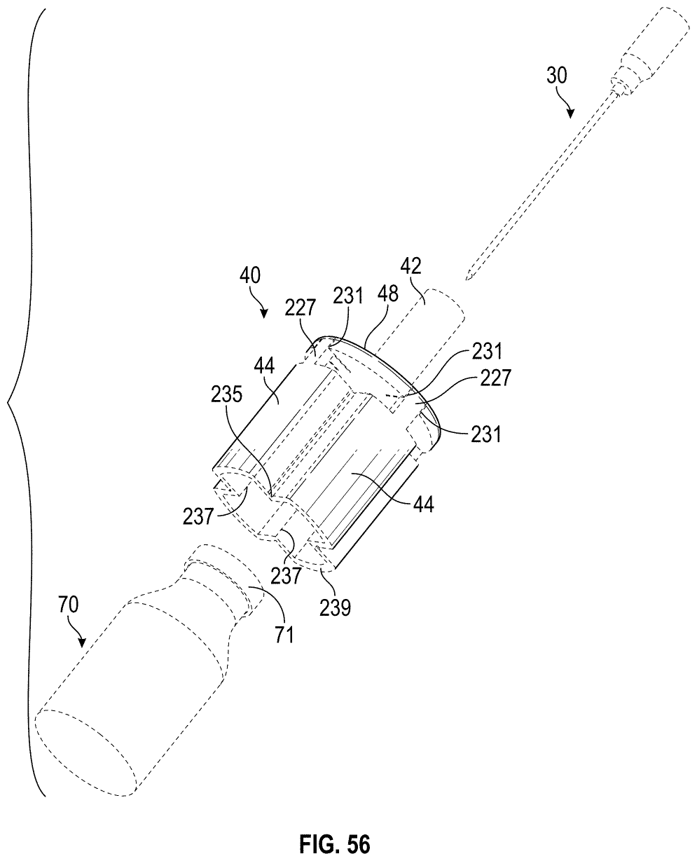

FIG. 56 shows a perspective view of the embodiment of guiding element 40 illustrated in FIG. 48 shown in an environment of use wherein guiding element 40 is configured to receive vials 70 and needles 30 of various dimensions.

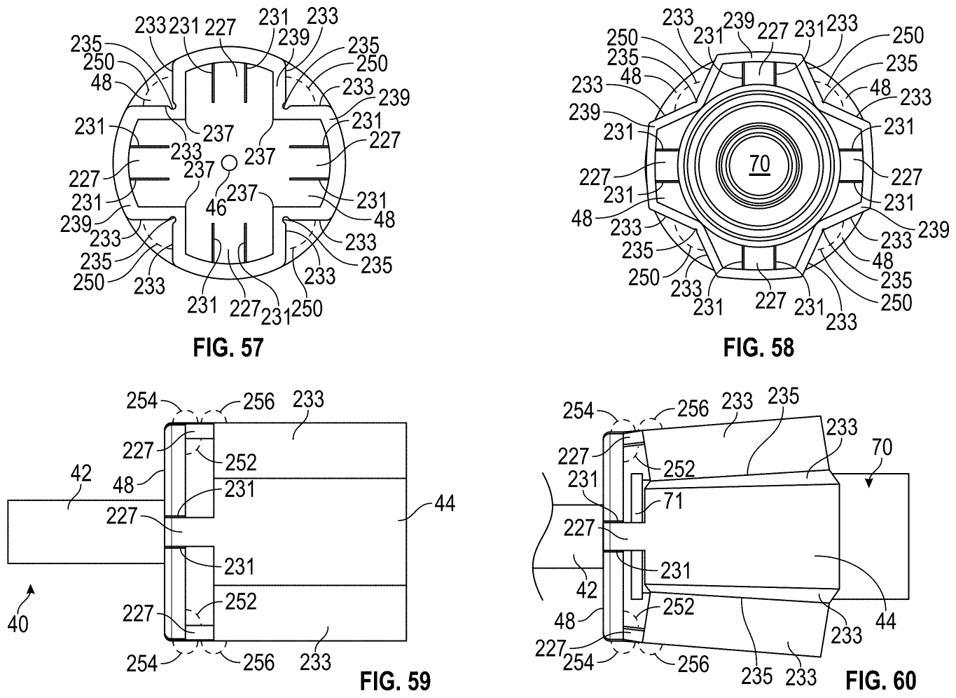

FIG. 57 shows a bottom view of the embodiment of guiding element 40 illustrated in FIG. 42, albeit without the shading. FIG. 57 also shows dot lined arcs 250 that represent the degree or angle between paired recessed portions 233. Both FIGS. 42 and 57 show paired recessed portions 233 situated such that arcs 250 are substantially 90 degrees. In one or more embodiments, pair recessed portions 233 may be situated such that arcs 250 are more or less than 90 degrees (that is, recessed portions 233 are more or less than 90 degrees in relation to each other).

FIG. 58 shows a bottom view of the embodiment of guiding element 40 illustrated in FIG. 57 with vial 70 having been inserted into it. As shown in FIG. 58, when vial 70 is inserted into guiding element 40, paired recessed portions 233 may move independently and resiliently articulate or extend away from each other such that the degree or angle of arcs 250 increases between each pair recessed portions 233. For example, in one or more embodiments, at rest (without a vial 70 inserted being inserted into guiding element 40) paired recessed portions 233 may be situated such that arcs 250 are substantially 90 degrees; however, once vial 70 has been inserted in guiding element 40, arcs 250 may increase to something more than 90 degrees (as shown in FIG. 58), with the amount of increase in degrees depending on the size of the vial 70 being inserted.

FIG. 59 shows a side view of the embodiment of guiding element 40 illustrated in FIG. 57. FIG. 59 shows that, in one or more embodiments, at rest (without a vial 70 inserted being inserted into guiding element 40), support arms 227 may be situated substantially 90 degrees in relation to the bottom surface of transition section 48 (as defined by dot lined arcs 252) and substantially 180 degrees or flush in relation to the exterior surface of barrel 44 and the exterior edge of transition section 48 (as defined by dot lined arcs 256 and 254, respectively). Although not shown, in one or more embodiments, support arms 227 may be situated such that arcs 252 are some degree other than substantially 90 degrees when barrel 44 is at rest and/or such that arcs 254 and/or 256 are some degree other than substantially 180 degrees when barrel 44 is at rest.

FIG. 60 shows a side view of the embodiment of guiding element 40 illustrated in FIG. 58 with vial 70 inserted into it. Depending on the size and configuration of vial 70 and depending on its location within barrel 44, in one or more embodiments, the support arms 227 may move independently and articulate or flex outwardly or inwardly (or engage in extension or flexion movements) as vial 70 is inserted into or pulled out of barrel 44. As shown in FIG. 60, in one or more embodiments, when vial 70 is inserted into barrel 44, support arms 227 may be situated such that the angles or degrees of arcs 252 are greater than substantially 90 degrees and arcs 254 and 256 are less than substantially 180 degrees. In one or more embodiments, recessed portions 233 may move independently and articulate or flex outwardly or inwardly (or engage in extension or flexion movements) at the channels 235. As shown in FIG. 60, in one or more embodiments, the diameter of one portion of barrel 44 may vary from the diameter of another portion of barrel 44, depending on the size and configuration of vial 70 and its location within barrel 44. For example, in one or more embodiments, when vial 70 is first inserted into barrel 44, the diameter of the barrel 44 at the vial's 70 entry point may be greater than the diameter of the barrel 44 proximal to transition section 48. In one or more embodiments, as vial 70 is pushed into barrel 44 the diameter of barrel 44 may expand accordingly and as vial 70 is pulled from barrel 44 the diameter of barrel 44 may contract accordingly.

Although not shown in FIGS. 57-60, in one or more embodiments, other embodiments of guiding element 40 contained herein, such as the embodiments of guiding element 40 illustrated and described in relation to FIGS. 48-56, 61-78, and 83-115, may be configured to possess substantially the same or similar functionality as described and shown in relation to FIGS. 57-60.

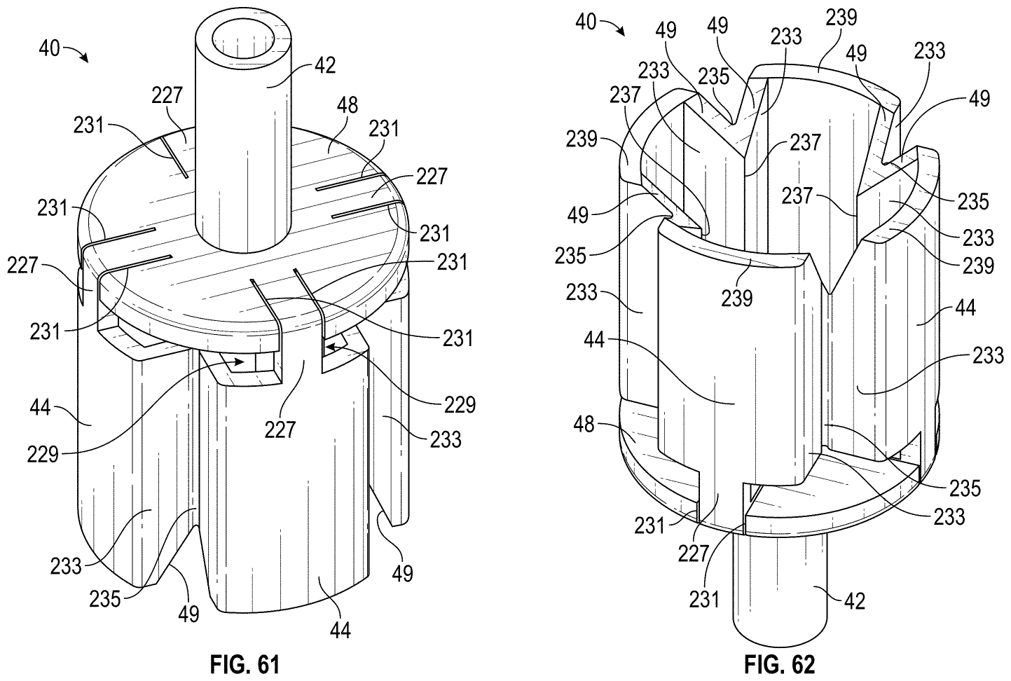

FIGS. 61-78 show alternative embodiments of guiding element 40 configured for at least the purpose of providing guiding element barrels 44 that easily receive, engage and release vials 70 of different sizes and that resiliently flex or expand and contract in order to house vials 70 of different sizes. In one or more embodiments, the embodiments of guiding element barrel 44 illustrated in FIGS. 61-78 may include angled edges 49 for guiding vial 70 into guiding element barrel 44. In one or more embodiments, the guiding elements 40 shown and described in relation to FIGS. 61-78 may include the structures and functionalities of the other embodiments of guiding element 40 shown and described herein (which structures and functionalities of said embodiments are hereby incorporated herein and applied to the guiding elements 40 shown and described in relation to FIGS. 61-78 by reference). For example, in one or more embodiments, the guiding elements 40 illustrated in FIGS. 61-78 may be designed with means to accommodate different sized vials 70. In one or more embodiments, the guiding elements 40 illustrated in FIGS. 61-78 may be used in conjunction with medication delivery devices 10 capable of automatically engaging various embodiments of volume limiter 50 for limiting the amount of medication that can be drawn from vials 70. In one or more embodiments, the guiding elements 40 illustrated in FIGS. 61-78 may be mixed and matched and substituted with other embodiments of guiding element 40 contained herein and used in conjunction with various embodiments of volume limiter 50 as part of medication delivery devices 10. In one or more embodiments, the guiding elements 40 illustrated in FIGS. 61-78 may include a transition section 48 between its guiding element shaft 42 and guiding element barrel 44 which may form a substantially right angle designed to engage the caps 71 of vials 70 and control the depth needles 30 may access vials 70 and aid in stabilizing vials 70 while they are secured in the guiding element barrel 44. In one or more embodiments, the guiding element barrels 44 illustrated in FIGS. 61-78 may guide needle 30 into substantially the center of vial 70 and secure or otherwise engage vial 70 while a user draws medication from it. In one or more embodiments, the guiding element shafts 42 illustrated in FIGS. 61-78 may house and protect needle 30 and other devices, and may prevent needle sticks. In one or more embodiments, once the medication has been extracted, the guiding elements 40 illustrated in FIGS. 61-78 may be removed from medication delivery device 10 to expose needle 30 so that the medication may be administered to the patient.

FIG. 61 shows a top perspective view of one embodiment of guiding element 40 which may include guiding element shaft 42, guiding element barrel 44, transition section 48, support arms 227, windows 229, slits 231, and recessed portions 233 of barrel 44. In one or more embodiments, like the guiding element 40 shown in FIG. 39, the guiding element 40 shown in FIG. 61 is configured to operably connect with the hub 32 or wings 33 of a needle 30. In one or more embodiments, said needle 30 may be configured to operably connect to a syringe tip 24 configured as a Luer-Lok syringe tip, such as shown in FIG. 1 (or said needle 30 may be configured to operably connect to a safety shield 60 that is configured to operably connect to a syringe tip 24 configured as a Luer-Lok syringe tip, such as shown in FIGS. 2 and 3). Although not shown in FIG. 61, in one or more embodiments, said needle 30 may be configured to operably connect to alternative syringe tips 24 or safety shields 60.

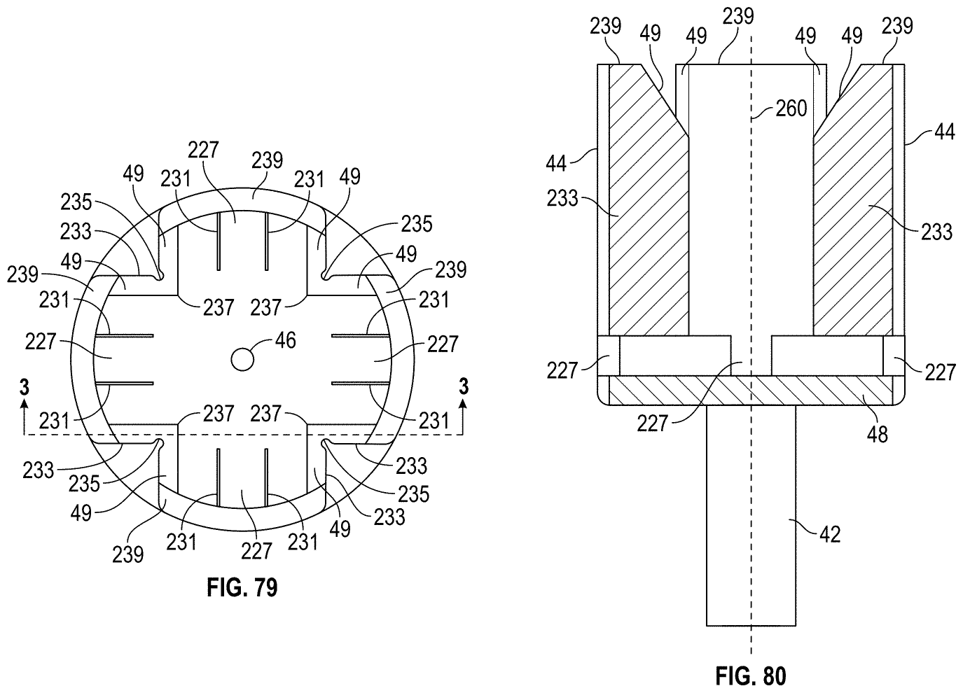

FIG. 62 shows a bottom perspective view of the embodiment of guiding element 40 illustrated in FIG. 61 including bottom barrel edge 239. As shown in FIG. 62, in one or more embodiments, at least some aspect of the bottom barrel edge 239 of each of the recessed portions 233 may be angled to some degree towards the bottom surface of the transition section 48. Each angled or slopped portion of the bottom barrel edge 239 of each recessed portion 233 is referred to herein as an angled edge 49. In one or more embodiments, the angled edges 49 may be substantially 45 degrees relative to the longitudinal axis 260 of barrel 44 (see FIGS. 80-82 for reference to the guiding element's 40 longitudinal axis 260). In one or more embodiments, the angled edges 49 may be more or less than 45 degrees relative to the longitudinal axis 260. At least one purpose of the angled edges 49 may be to allow a user to insert vials 70 of different sizes more easily into barrel 44. In one or more embodiments, like the function of the angled edges 49 of the embodiments of guiding element 40 illustrated in FIGS. 28A-33, the function of the angled edges 49 of the embodiments of guiding element 40 illustrated in FIGS. 61-78 may be for guiding vial 70 into guiding element barrel 44 (such as shown and described in relation to FIGS. 79-82).

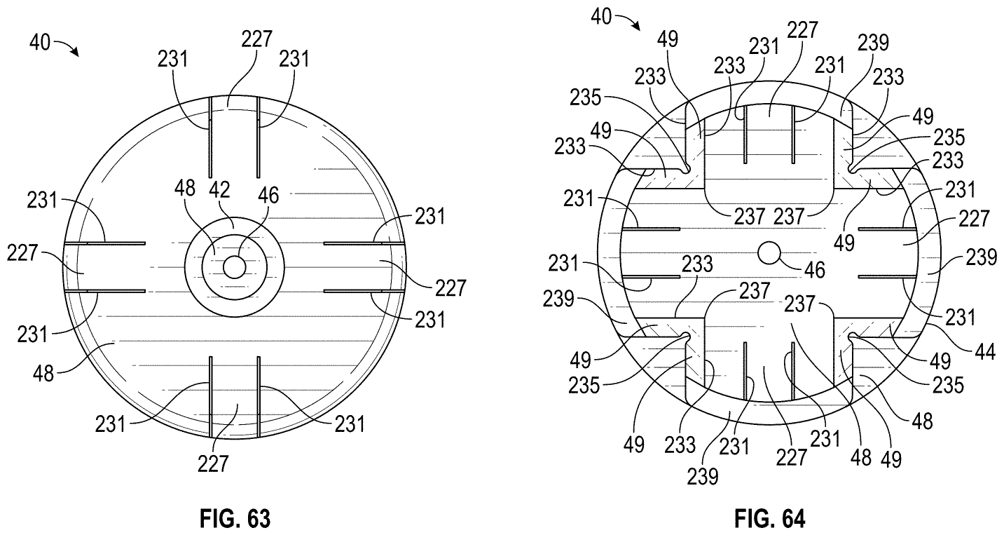

FIG. 63 shows a top view of the embodiment of guiding element 40 illustrated in FIG. 61. As shown in FIG. 63, in one or more embodiments, guiding element 40 may include eight slits 231, four support arms 227, and an opening 46 through which needle 30 is inserted.

FIG. 64 shows a bottom view of the embodiment of guiding element 40 illustrated in FIG. 61 including recessed portions 233, bottom barrel edge 239 and channels 235. As shown in FIG. 64, in one or more embodiments, each recessed portion 233 may include an angled edge 49. In one or more embodiments, paired recessed portions 233 may be bent inward and meet to form a channel 235 on the exterior surface of barrel 44 and to form a barrel engagement surface 237 on the interior surface of barrel 44. In one or more embodiments, paired recessed portions 233 with angled edges 49 meet to form substantially a sloping “V” formation, with the tip of the “V” formation defining the barrel engagement surfaces 237. As indicated above, at least one purpose of the barrel engagement surfaces 237 may be to engage the side of a vial 70 when it is inserted into guiding element barrel 44.

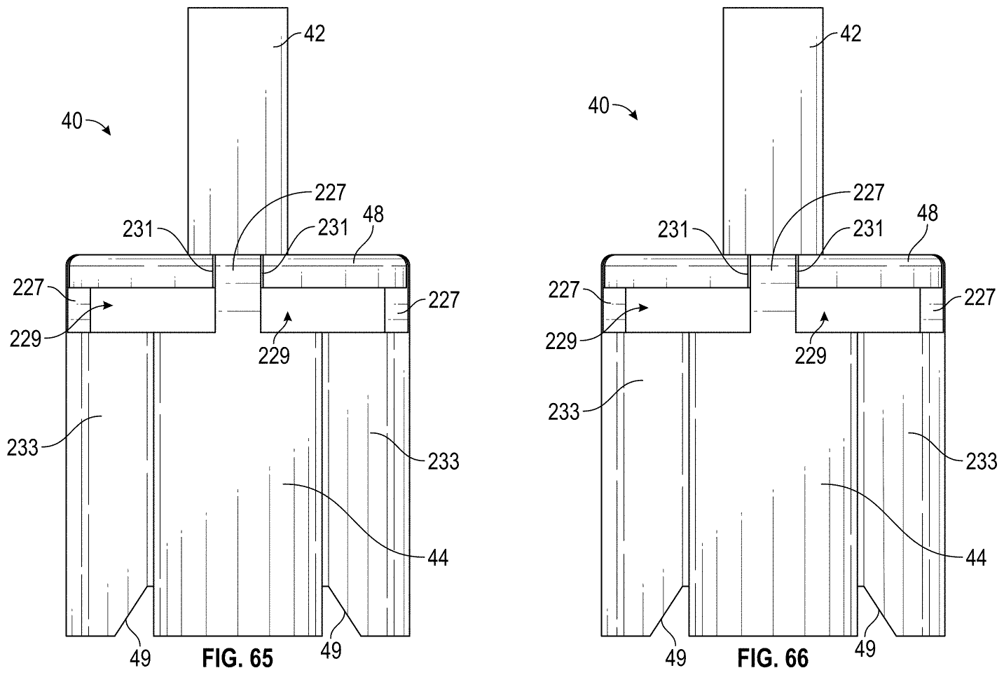

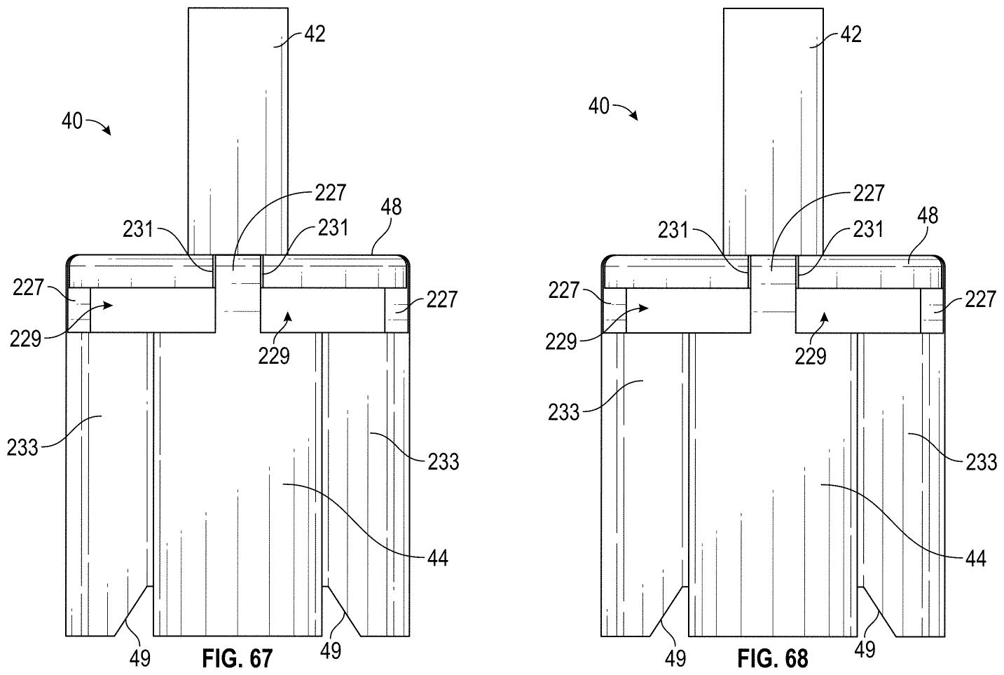

FIG. 65 shows a right side view of the embodiment of guiding element 40 illustrated in FIG. 61. FIG. 66 shows a left side view of the embodiment of guiding element 40 illustrated in FIG. 61. FIG. 67 shows a front view of the embodiment of guiding element 40 illustrated in FIG. 61. FIG. 68 shows a rear view of the embodiment of guiding element 40 illustrated in FIG. 61. As shown in FIGS. 65-68, in one or more embodiments, guiding element 40 may include four windows 229. In one or more embodiments, the bottom surface of transition section 48 and the interior surface of guiding element barrel 44 may be visible through windows 229.

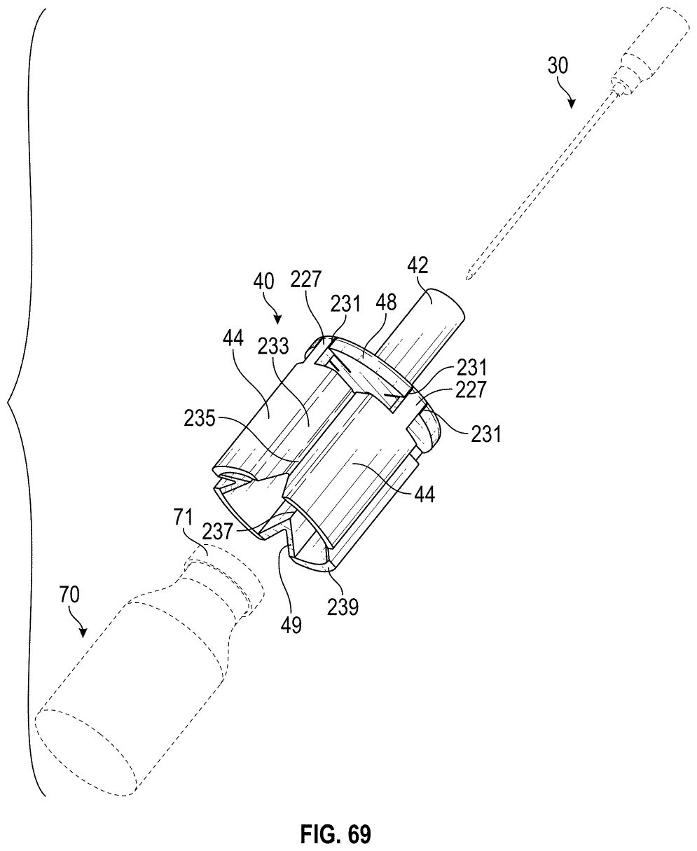

FIG. 69 shows a perspective view of the embodiment of guiding element 40 illustrated in FIG. 61 shown in an environment of use wherein guiding element 40 is configured to receive vials 70 and needles 30 of various dimensions.

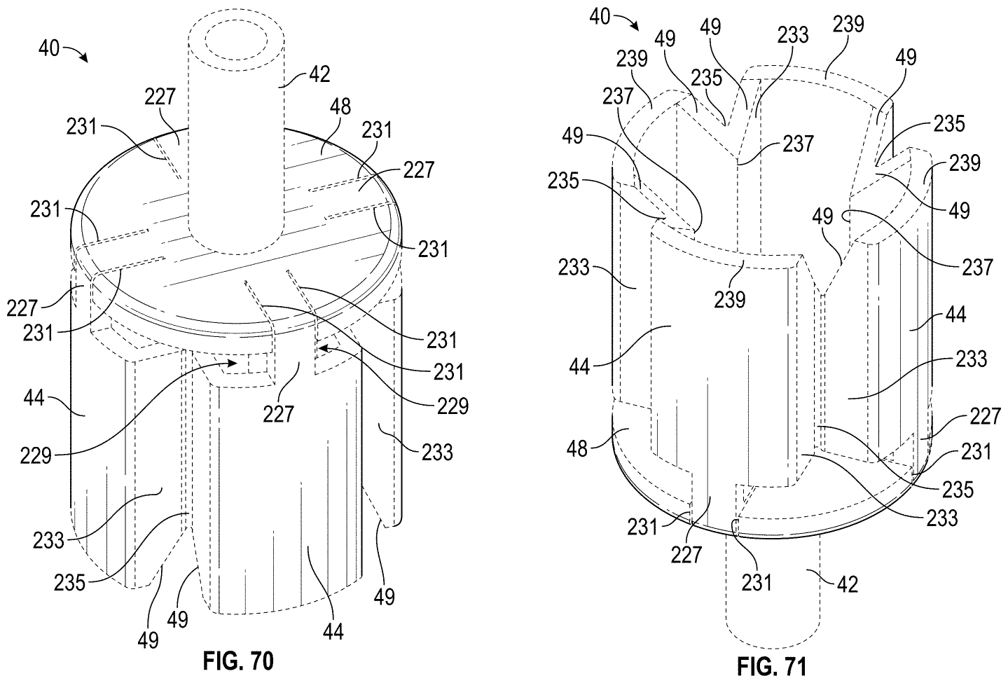

FIGS. 70-78 show that the configuration of components of the embodiment of guiding element 40 illustrated in FIGS. 61-69 may vary. For example, although FIGS. 61-69 show guiding element 40 including four support arms 227, four windows 229, eight slits 231, eight recessed portions 233, four channels 235, and four barrel engagement surfaces 237, in one or more embodiments, more or less support arm 227, windows 229, slits 231, recessed portions 233, channels 235, and barrel engagement surfaces 237 may be provided and their dimensions and locations on guiding element 40 may vary. For example, in one or more embodiments, guiding element 40 may include three support arms 227, three windows 229, six slits 231, and six recessed portions 233 bent inward meeting to form three channels 235 on the exterior surface and three barrel engagement surfaces 237 on the interior surface of barrel 44. Although FIGS. 61-69 show slits 231 cut to a certain depth and at a certain angle, in one or more embodiments, slits 231 may be made by a vertical cut or an angled cut or a multi-angled cut through the transition section 48 and, in one or more embodiments, the depth of the cuts towards the center of transition section 28 from its circumference may vary. Although FIGS. 61-69 show the bottom barrel edge 239 resembling a “+” or plus sign formation from a bottom view, in one or more embodiments, the configuration of guiding element barrel 44 may assume alternative formations, symmetrical or non-symmetrical, such as resembling a “Y” formation from a bottom view. In one or more embodiments, the possible modifications to guiding element 40 are not limited to the aspects of guiding element 40 that are dot lined in FIGS. 70-78.

FIG. 70 shows a top perspective view of one embodiment of the guiding element 40 illustrated in FIG. 61 with various components dot lined. As shown by the dotted lines in FIG. 70, in one or more embodiments, the thickness, length, location, and other dimensions and configurations of shaft 42, support arms 227, slits 231, and recessed portions 233 may vary. As shown by the dotted lines in FIG. 70, in one or more embodiments, the diameter, thickness and other dimensions and configurations of transition section 48 may vary. As shown by the dotted lines in FIG. 70, in one or more embodiments, guiding element 40 may not include support arms 227, windows 229, slits 231, or recessed portions 233 (such as shown in FIGS. 1-3, 5A-5B, and 6-8B).

FIG. 71 shows a bottom perspective view of the embodiment of guiding element 40 illustrated in FIG. 70. As shown by the dotted lines in FIG. 71, in one or more embodiments, the angle, gradualness, or sharpness at which recessed portions 233 of barrel 44 recess inward from the walls of barrel 44 may vary. For example, in one or more embodiments, the recessed portions 233 may recess inward from the walls of barrel 44 at substantially 90 degrees, less than 90 degrees, or more than 90 degrees. In one or more embodiments, any two recessed portions 233 that form a pair may be situated such that they are substantially 90 degrees, less than 90 degrees, or more than 90 degrees in relation to each other. As shown by the dotted lines in FIG. 71, in one or more embodiments, the thickness, length, diameter and other dimensions and configurations of the guiding element barrel's 44 wall may vary.

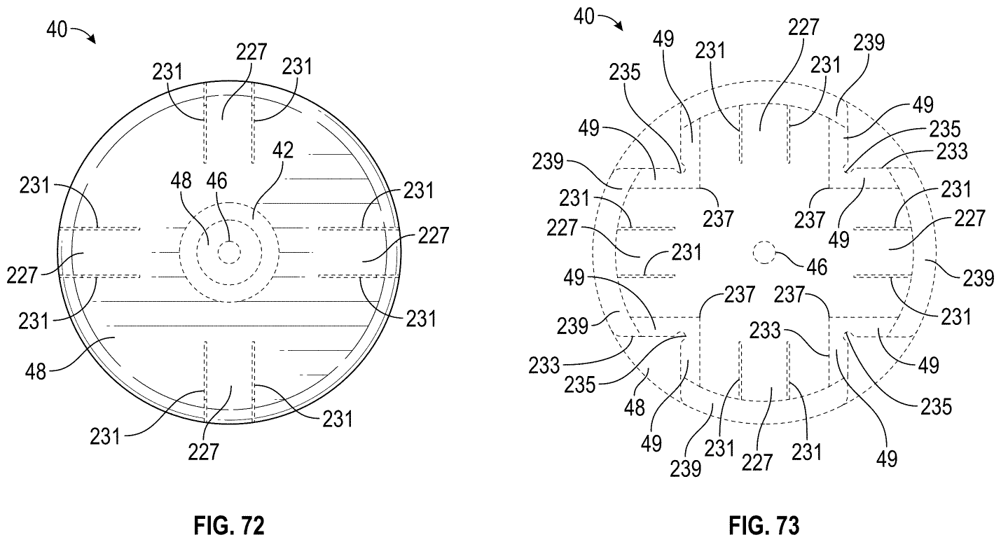

FIG. 72 shows a top view of the embodiment of guiding element 40 illustrated in FIG. 70. Although FIGS. 61-69 show shaft 42 and opening 46 at certain diameters and locations on guiding element 40, as shown by the dotted lines in FIG. 72, in one or more embodiments, the diameters and locations of shaft 42 and opening 46 may vary depending on the size of the needles 30 that guiding element 40 is designed to engage. As shown by the dotted lines in FIG. 72, in one or more embodiments, guiding element 40 may not include any slits 231.

FIG. 73 shows a bottom view of the embodiment of guiding element 40 illustrated in FIG. 70. As shown by the dotted lines in FIG. 73, in one or more embodiments, the profile and other dimensions and configurations of channels 235 may vary. For example, in one or more embodiments, the channels 235 may assume a rounded profile (such as in FIGS. 39-47), a sharp profile (such as in FIGS. 106-107 and 109), an angled profile, a multi-angled profile, a multi-surfaced profile, or other profile configuration. In one or more embodiments, the angle and sharpness at which recessed portions 233 recess inward from the walls of barrel 44 and the characteristics of the channels' 235 profile, may be modified in order to provide the barrel 44 enough flexibility, rigidity, and resiliency to engage vials 70 of various dimensions. In one or more embodiments, guiding element 40 may not include channels 235 (such as shown in FIGS. 1-3, 5A-5B, and 6-8B).

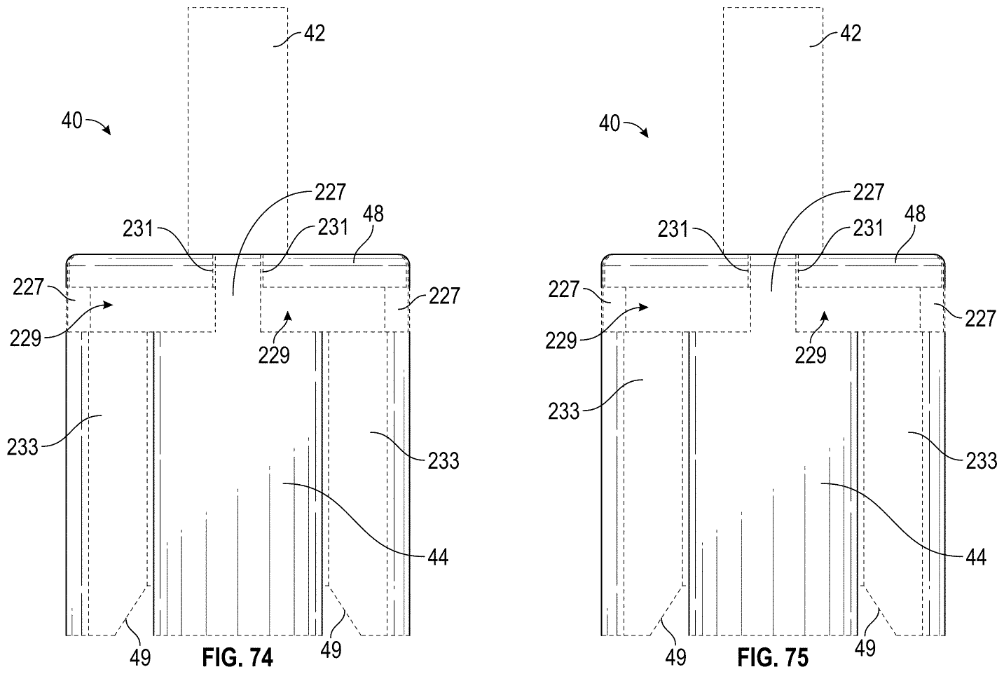

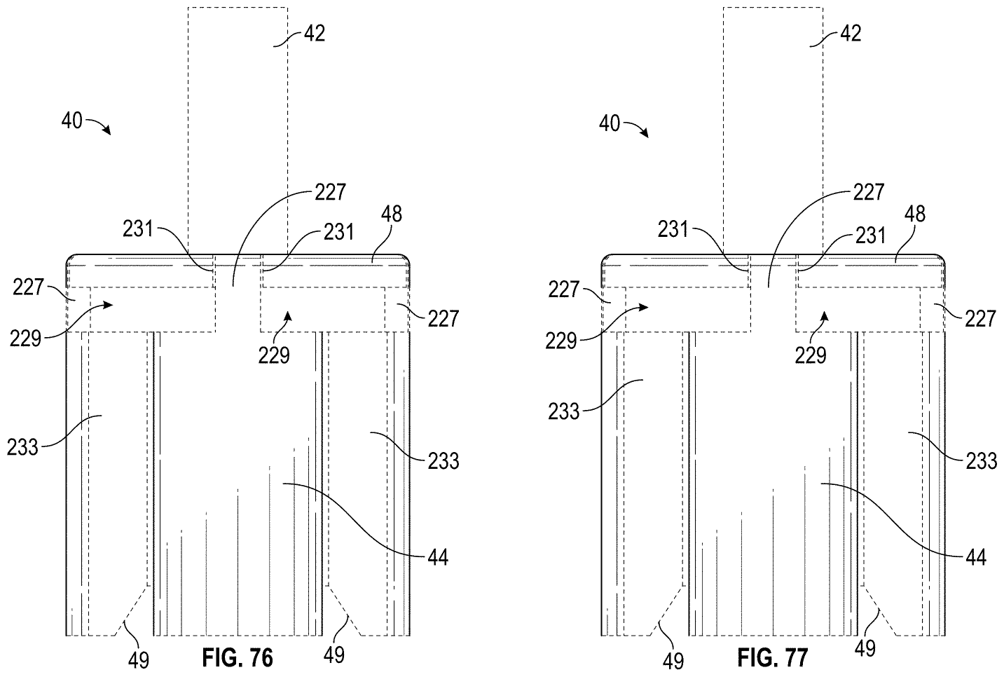

FIG. 74 shows a right side view of the embodiment of guiding element 40 illustrated in FIG. 70. FIG. 75 shows a left side view of the embodiment of guiding element 40 illustrated in FIG. 70. FIG. 76 shows a front view of the embodiment of guiding element 40 illustrated in FIG. 70. FIG. 77 shows a rear view of the embodiment of guiding element 40 illustrated in FIG. 70. As shown by the dotted lines in FIGS. 74-77, in one or more embodiments, the number, height, width and other dimensions and configurations of windows 229, slits 231, and support arms 227 may vary.

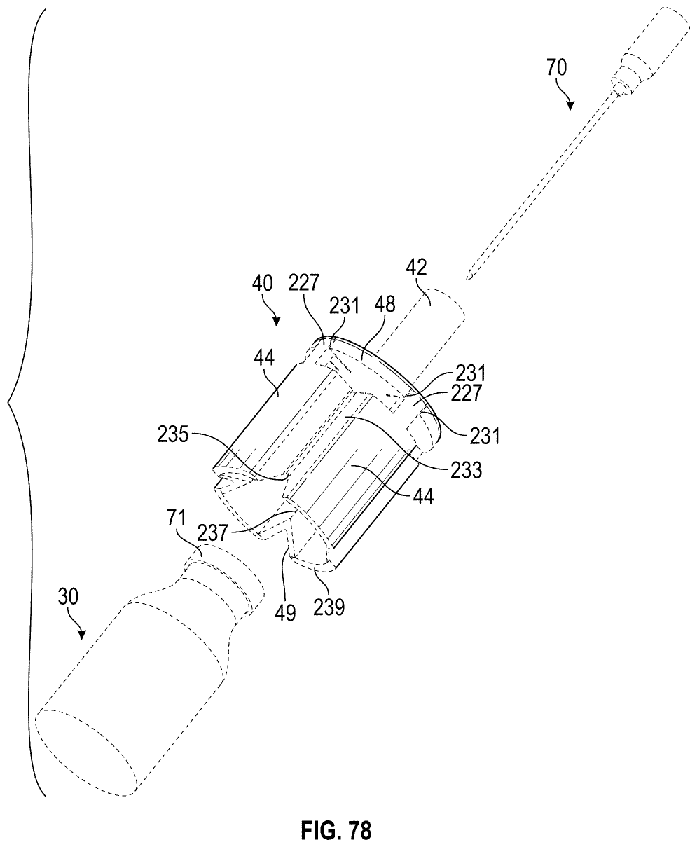

FIG. 78 shows a perspective view of the embodiment of guiding element 40 illustrated in FIG. 70 shown in an environment of use wherein guiding element 40 is configured to receive vials 70 and needles 30 of various dimensions.

FIG. 79 shows a bottom view of the embodiment of guiding element 40 illustrated in FIG. 64, albeit without the shading.

FIG. 80 shows a cross sectional view along lines 3-3 of the embodiment of guiding element 40 illustrated in FIG. 79 including a longitudinal axis 260. As indicated above, in one or more embodiments, paired recessed portions 233 with angled edges 49 meet to form substantially a sloping “V” formation, with the tip of the “V” formation defining the barrel engagement surfaces 237.

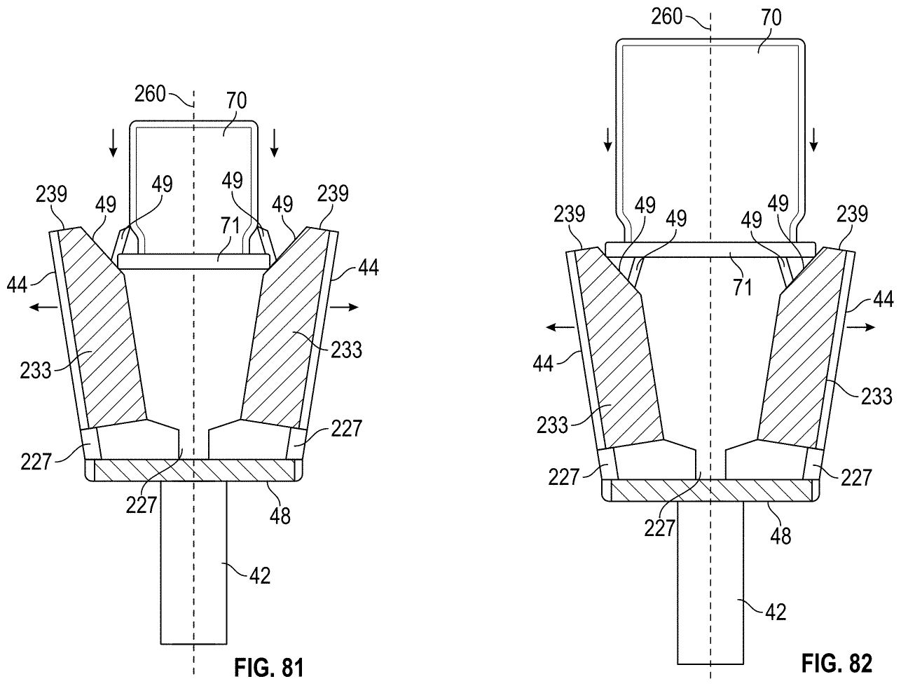

FIG. 81 shows a cross sectional view along lines 3-3 of the embodiment of guiding element 40 illustrated in FIG. 79 with the angled edges 49 (of the recessed portions 233 of barrel 44) engaging vial 70. As indicated above, at least one purpose of the angled edges 49 may be to allow a user to insert vials 70 of different sizes more easily into barrel 44. As shown in FIG. 81, in one or more embodiments, as a vial 70 is inserted into barrel 44 and comes into contact with the angled edges 49, the force on the angled edges 49 causes the recessed portions 233 to be push outwardly, away from the barrel's 44 longitudinal axis 260, thereby causing the circumference of the barrel 44 to expand and more easily allowing the user to insert the vial 70 into the barrel 44. The inclusion of the angled edges 49 into barrel 44, in one or more embodiments, may reduce or eliminate the force required to insert a vial 70 into barrel 44 (such as may be required when inserting a vial 70 into the embodiments of barrel 44 that do not have angled edges 49, such as illustrated in FIGS. 39-60).

FIG. 82 shows a cross sectional view along lines 3-3 of the embodiment of guiding element 40 illustrated in FIG. 79 with the angled edges 49 engaging a larger vial 70 (as compared to the size of the vial 70 shown in FIG. 81). As shown in FIG. 82, in one or more embodiments, vials 70 of different sizes may initially engage the surface of the angled edges 49 at different locations.

Although not shown in FIGS. 79-82, in one or more embodiments, other embodiments of guiding element 40 contained herein, such as the embodiments of guiding element 40 illustrated and described in relation to FIGS. 39-60, 70-78, and 83-115, may be configured to possess substantially the same or similar functionality as described and shown in relation to FIGS. 79-82.

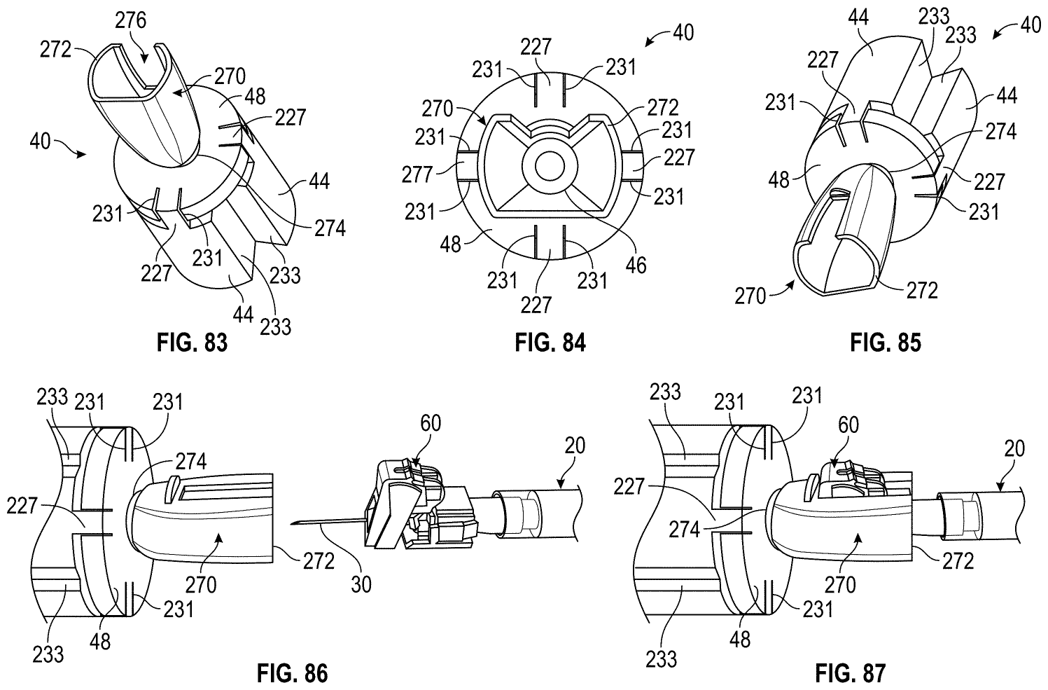

FIGS. 83-95 illustrate that, in one or embodiments, guiding element 40 may be configured with various devices that enable it to operably connect to syringes 20 that may include various components. For example, in one or more embodiments, guiding element 40 may be configured with a receiving boot designed to receive needles 30 that may include a safety shield. In one or more embodiments, the guiding elements 40 shown and described in relation to FIGS. 83-95 may include the structures and functionalities of the other embodiments of guiding element 40 shown and described herein (which structures and functionalities of said embodiments are hereby incorporated herein and applied to the guiding elements 40 shown and described in relation to FIGS. 83-95 by reference). For example, in one or more embodiments, the guiding elements 40 illustrated in FIGS. 83-95 may be designed with means to accommodate different sized vials 70. In one or more embodiments, the guiding elements 40 illustrated in FIGS. 83-95 may be used in conjunction with medication delivery devices 10 capable of automatically engaging various embodiments of volume limiter 50 for limiting the amount of medication that can be drawn from vials 70. In one or more embodiments, the guiding elements 40 illustrated in FIGS. 83-95 may be mixed and matched and substituted with other embodiments of guiding element 40 contained herein and used in conjunction with various embodiments of volume limiter 50 as part of medication delivery devices 10. In one or more embodiments, the guiding elements 40 illustrated in FIGS. 83-95 may include a transition section 48 between its guiding element shaft 42 and guiding element barrel 44 which may form a substantially right angle designed to engage the caps 71 of vials 70 and control the depth needles 30 may access vials 70 and aid in stabilizing vials 70 while they are secured in the guiding element barrel 44. In one or more embodiments, the guiding element barrels 44 illustrated in FIGS. 83-95 may guide needle 30 into substantially the center of vial 70 and secure or otherwise engage vial 70 while a user draws medication from it. In one or more embodiments, the guiding element shafts 42 illustrated in FIGS. 83-95 may house and protect needle 30 and other devices, and may prevent needle sticks. In one or more embodiments, once the medication has been extracted, the guiding elements 40 illustrated in FIGS. 83-95 may be removed from medication delivery device 10 to expose needle 30 so that the medication may be administered to the patient.

FIG. 83 shows a perspective view of one embodiment of a receiving boot 270 operably connected to one embodiment of guiding element 40. In one or more embodiments, the receiving boot 270 shown in FIG. 83 is configured to receive a syringe 20 with a sliding safety shield 60 that is configured as a Monoject Magellan safety device. Although not shown in FIG. 83, receiving boot 270 may be configured to receive a syringe 20 with an alternative sliding safety shield 60 configuration. In one or more embodiments, receiving boot 270 may include a receiving end 272 that is configured to receive and engage sliding safety shield 60. In one or more embodiments, receiving boot 270 may include an attachment end 274 that is operably connected or connectable to the guiding element 40. In one or more embodiments, receiving boot 270 may also include a groove 276 or some other structural adaptation for receiving sliding safety shield 60. In one or more embodiments, receiving boot 270 may be integral to the guiding element 40 (such as to its shaft 42 or, as shown in FIG. 83, directly to its transition section 48 in situations in which guiding element 40 is configured without a shaft 42). In one or more embodiments, receiving boot 270 may be releasably connectable or permanently connected to guiding element 40 directly to transition section 48, shaft 42, or some other aspect of guiding element 40.

FIG. 84 shows an alternative view of the embodiments of receiving boot 270 and guiding element 40 illustrated in FIG. 83, albeit slits 231 assume an alternative configuration. FIG. 84 shows opening 46, where needle 30 may be inserted.

FIG. 85 shows an alternative perspective view of the embodiments of receiving boot 270 and guiding element 40 illustrated in FIG. 83.

FIG. 86 shows an embodiment of needle 30 configured with one embodiment of a sliding safety shield 60 that is configured as a Monoject Magellan safety device. FIG. 86 shows one embodiment of needle 30 approaching the receiving boot 270 illustrated in FIG. 83. FIG. 86 shows an embodiment of a 25 gauge needle 30 about ⅝ of an inch long that is configured for subcutaneous injections, with a Monoject Magellan safety device operably connected to it. In one or more embodiments, sliding safety shields 60, such as the Monoject Magellan safety device illustrated in FIG. 86, may have an articulating mechanism that allows a user to extend it such that it fully covers the needle 30. FIG. 86 shows one embodiment of an articulating mechanism of sliding safety shield 60 in a retracted position.

FIG. 87 shows the embodiment of the sliding safety shield 60 illustrated in FIG. 86 inserted into receiving boot 270. In one or more embodiments, syringe 20 may be removed by a user from receiving boot 270 as desired.

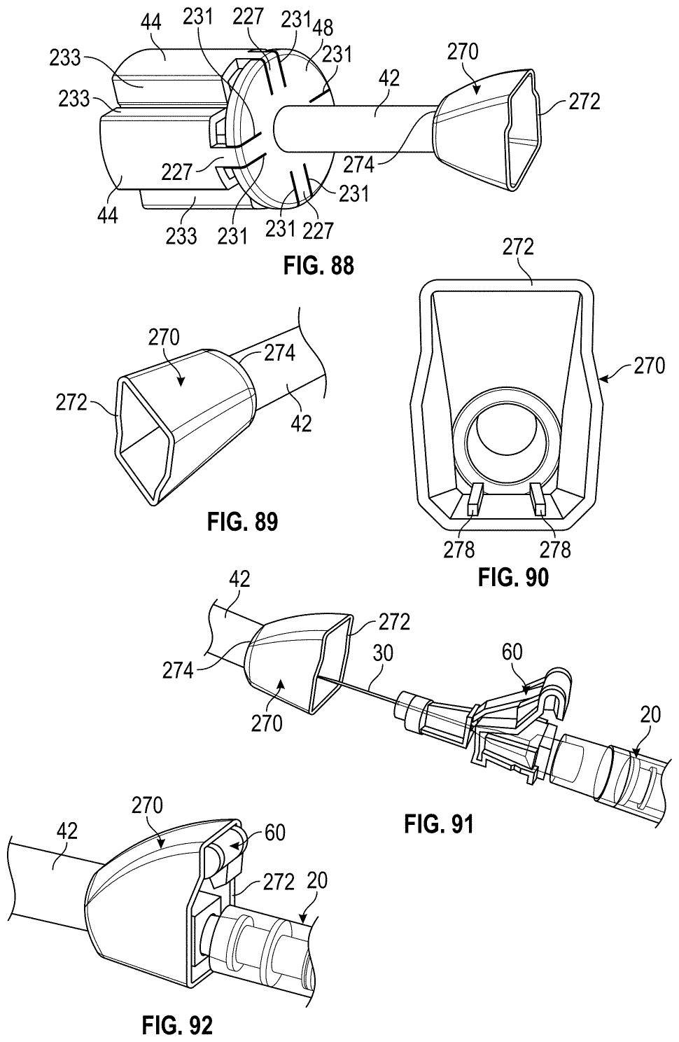

FIG. 88 shows a perspective view of an alternative embodiment of receiving boot 270 operably connected to the shaft 42 of one embodiment of guiding element 40. In one or more embodiments, the receiving boot 270 shown in FIG. 88 is configured to receive a syringe 20 with a sliding safety shield 60 that is configured as a Becton, Dickinson and Company (also known as “BD”) SafetyGlide safety device. Although not shown in FIG. 88, receiving boot 270 may be configured to receive a syringe 20 with an alternative sliding safety shield 60 configuration. As indicated above, in one or more embodiments, receiving boot 270 may include a receiving end 272 that is configured to receive and engage sliding safety shield 60. In one or more embodiments, receiving boot 270 may include an attachment end 274 that is operably connected or connectable to the guiding element 40. Unlike the embodiment of receiving boot 270 illustrated in FIGS. 83-87, as shown in FIG. 88, in one or more embodiments, receiving boot 270 may not include groove 276. Although not shown in FIGS. 83-92, in one or more embodiments, the shape and other dimensions and configurations of receiving boot 270 may be modified to receive sliding safety shields 60 of various dimensions and configurations other than and/or in addition to the BD SafetyGlide and Monoject Magellan safety devices shown herein.

FIG. 89 shows a bottom perspective view of the embodiment of receiving boot 270 illustrated in FIG. 88.

FIG. 90 shows a front view of the embodiment of receiving boot 270 illustrated in FIG. 88, albeit with rails 278. As shown in FIG. 90, in one or more embodiments, receiving boot 270 may include rails 278 or other structural modifications to aid in receiving and engaging sliding safety shields 60 of various dimensions and configurations.

FIG. 91 shows a perspective view of the embodiment of the receiving boot 270 illustrated in FIG. 88 being approached by a needle 30 configured with an embodiment of sliding safety shield 60 that is configured as a BD SafetyGlide safety device. FIG. 91 shows an embodiment of a 23 gauge needle 30 about 1.5 inches long configured for intramuscular injections, with a BD SafetyGlide safety device operably connected to it. In one or more embodiments, sliding safety shields 60, such as the BD SafetyGlide safety device illustrated in FIG. 91, may have an articulating mechanism that allows a user to extend it such that it fully covers the needle 30. FIG. 91 shows one embodiment of an articulating mechanism of sliding safety shield 60 in a retracted position.

FIG. 92 shows the embodiment of the sliding safety shield 60 illustrated in FIG. 91 inserted into the receiving boot 270 illustrated in FIG. 91. In one or more embodiments, syringe 20 may be removed by a user from receiving boot 270 as desired.

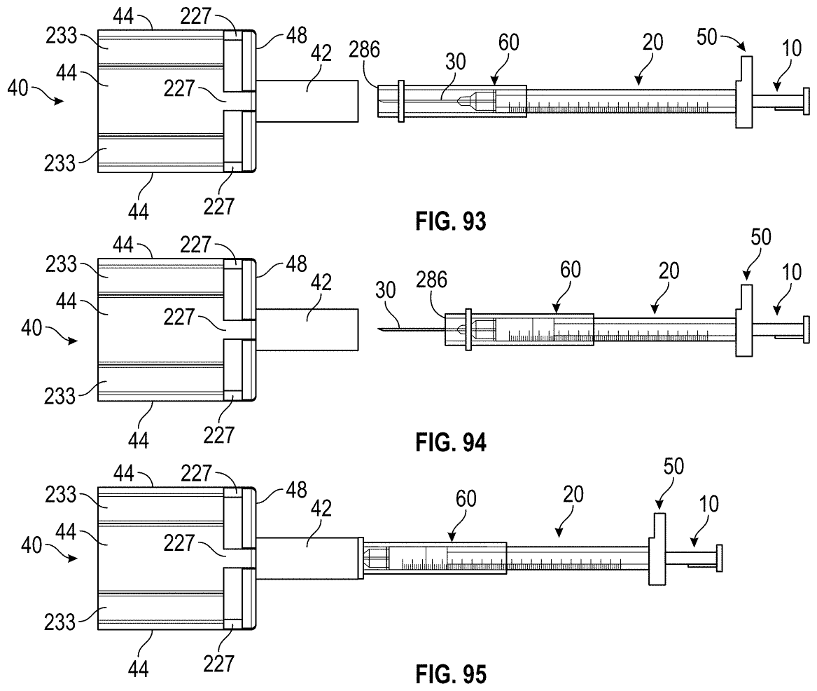

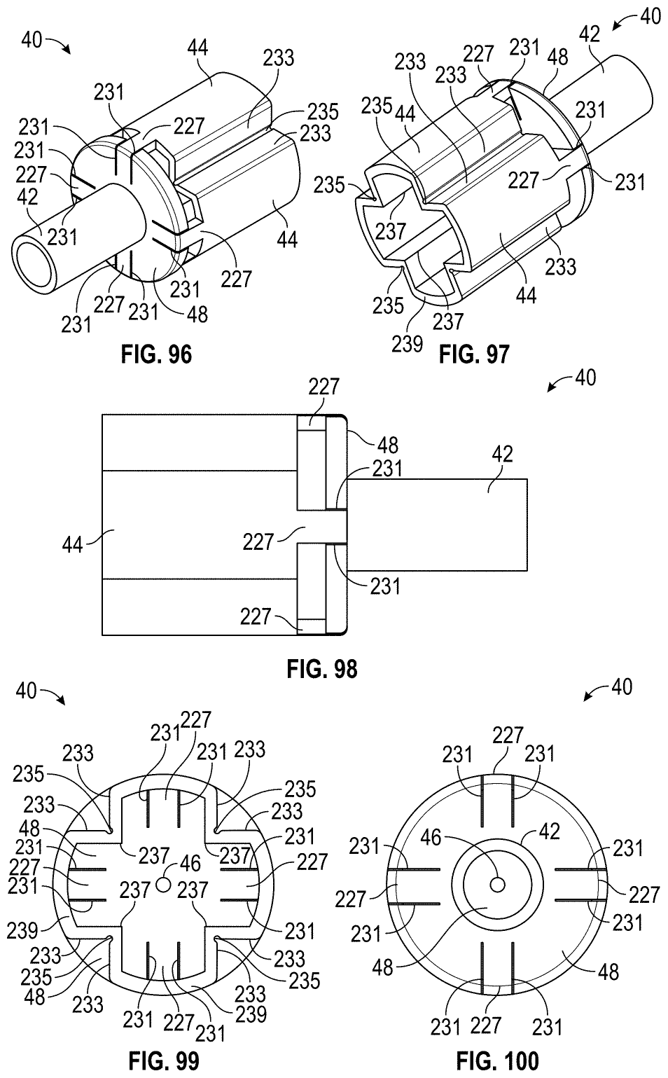

FIG. 93 shows one embodiment of syringe 20 with a sliding safety shield 60 that is integrally configured to syringe 20. In one or more embodiments, the sliding safety shield 60 shown in FIG. 93 is configured as an Ultimed UtliCare safety device and the guiding element 40 shown in FIG. 93 is configured to receive such. As shown in FIG. 93, in one or more embodiments, no additional receiving boot 270 is required to be operably connected to guiding element 40 because the diameter of the opening of its shaft 42 is configured to receive an inserting end 286 of sliding safety shield 60 (see the embodiments of guiding element 40 illustrated in FIGS. 96-110 for additional examples). Although not shown in FIG. 93, in one or more embodiments, the diameter of shaft 42 may be increased or decrease to fit virtually any sized syringe 20 or sliding safety shields 60. Although not shown in FIG. 93, in one or more embodiments, guiding element 40 may be configured to receive a syringe 20 with a sliding safety shield 60 that is configured differently than an Ultimed UtliCare safety device.

FIG. 94 shows the embodiments of syringe 20 and sliding safety shield 60 illustrated in FIG. 93, albeit sliding safety shield 60 has been retracted to expose needle 30.

FIG. 95 shows the embodiment of syringe 20 illustrated in FIG. 93 inserted into guiding element 40.

Although not shown in FIGS. 83-95, in one or more embodiments, other embodiments of guiding element 40 contained herein, such as the embodiments of guiding element 40 illustrated and described in relation to FIGS. 39-78, and 96-115, may be configured to possess substantially the same or similar functionality as described and shown in relation to FIGS. 83-95.

FIGS. 96-115 show various embodiments of guiding element 40 featuring, among other things, differently dimensioned and configured barrels 44 and shafts 42 for at least the purpose of housing vials 70 and needles 30 of various sizes and being able to operably connect to devices of various sizes and syringes 20 of various sizes and configurations. In one or more embodiments, the circumference of barrel 44 may be large and its walls may be flexible or semi-flexible to accommodate a wide variety of vial 70 sizes. For example, in one or more embodiments, a single barrel 44 may be configured to house at least between 1 mL and 10 mL vials 70 (or even greater than 10 mL vials 70). Alternatively, in one or more embodiments, the circumference of barrel 44 may be smaller and more compact and its walls may be less flexible or semi-flexible to accommodate a particular vial 70 size or sizes. For example, in one or more embodiments, a single barrel 44 may be configured to house only 1 mL vials 70. In one or more embodiments, the length of barrel 44 may vary depending on the size of the vials 70 it is designed to engage or to satisfy some other need. In one or more embodiments, the length of shaft 42 may be long to accommodate long needles 30. Alternatively, in one or more embodiments, the length of shaft 42 may be shorter to accommodate short needles 30. In one or more embodiments, the shaft 42 of guiding element 40 may be configured to house needles 30 less than ⅜ of an inch long or up to and even longer than 3½ inches. For example, in one or more embodiments, guiding element 40 may be configured to operably connect to syringes 20 designed for intramuscular injections (with needles 30 ⅞ of an inch to 1½ inches long), subcutaneous injections (with needles 30 ½ of an inch to ⅝ inches long), or intradermal injections (with needles 30 ⅜ of an inch to ¾ of an inch long). In one or more embodiments, the diameter of shaft 42 may be wide to accommodate syringes 20 with various devices (such as the embodiments of guiding element 40 shown and described in reference to FIGS. 93-95 that may be configured to operably connect to a safety shield). Alternatively, in one or more embodiments, the diameter of shaft 42 may be smaller to engage directly with the hub 32 or wings 33 of a needle 30 (see the embodiments of guiding element 40 shown and described in reference to FIGS. 1-9 and 111-115). In one or more embodiments, guiding element 40 may be configured to house needles 30 of various gauges. For example, in one or more embodiments, guiding element 40 may be configured to house needles 30 with gauges less than 6 and up to and even more than 32 gauged needles 30. In one or more embodiments, the diameter of opening 46 of guiding element barrel 44 may be modified to receive needles 30 with various gauges/diameters. In one or more embodiments, guiding element 40 may be configured without a shaft 42. In one or more embodiments, guiding element 40 may include various sized barrels 44 that may be mixed and matched with various sized shafts 42 depending on the need. In one or more embodiments, the exact dimensions and configurations of the shaft 42 and barrel 44 may be determined by the versatility needed or not needed. The FIGS. 96-115 are not necessarily to scale in relation to each other. Although not shown in FIGS. 96-115, in one or more embodiments, the shaft 42 of guiding element 40 may be configured to operably connect to syringes 20 with slip tips, eccentric tips, or catheter tips. In one or more embodiments, guiding element 40 may be configured to operably connect to syringes 20 made by various manufactures. In one or more embodiments, guiding element 40 may be configured to operably connect to syringes 20 that include various devices of various sizes and configurations, such as safety shields 60.

In one or more embodiments, the guiding elements 40 shown and described in relation to FIGS. 96-115 may include the structures and functionalities of the other embodiments of guiding element 40 shown and described herein (which structures and functionalities of said embodiments are hereby incorporated herein and applied to the guiding elements 40 shown and described in relation to FIGS. 96-115 by reference). For example, in one or more embodiments, the guiding elements 40 illustrated in FIGS. 96-115 may be designed with means to accommodate different sized vials 70. In one or more embodiments, the guiding elements 40 illustrated in FIGS. 96-115 may be used in conjunction with medication delivery devices 10 capable of automatically engaging various embodiments of volume limiter 50 for limiting the amount of medication that can be drawn from vials 70. In one or more embodiments, the guiding elements 40 illustrated in FIGS. 96-115 may be mixed and matched and substituted with other embodiments of guiding element 40 contained herein and used in conjunction with various embodiments of volume limiter 50 as part of medication delivery devices 10. In one or more embodiments, the guiding elements 40 illustrated in FIGS. 96-115 may include a transition section 48 between its guiding element shaft 42 and guiding element barrel 44 which may form a substantially right angle designed to engage the caps 71 of vials 70 and control the depth needles 30 may access vials 70 and aid in stabilizing vials 70 while they are secured in the guiding element barrel 44. In one or more embodiments, the guiding element barrels 44 illustrated in FIGS. 96-115 may guide needle 30 into substantially the center of vial 70 and secure or otherwise engage vial 70 while a user draws medication from it. In one or more embodiments, the guiding element shafts 42 illustrated in FIGS. 96-115 may house and protect needle 30 and other devices, and may prevent needle sticks. In one or more embodiments, once the medication has been extracted, the guiding elements 40 illustrated in FIGS. 96-115 may be removed from medication delivery device 10 to expose needle 30 so that the medication may be administered to the patient.

FIGS. 96-110 show various embodiments of guiding element 40 wherein no additional receiving boot 270 is required to be operably connected to said guiding elements 40 because the diameter of the openings of their shafts 42 are configured to receive inserting ends 286 of sliding safety shields 60. In one or more embodiments, the guiding elements illustrated in FIGS. 96-110 receive sliding safety shields 60 configured as Ultimed UtliCare safety devices. In one or more embodiments, the guiding elements illustrated in FIGS. 96-110 may be configured to receive syringes 20 that include sliding safety shields 60 of some other configuration.

FIG. 96 shows a top perspective view of one embodiment of guiding element 40 configured to receive a syringe 20 that includes a sliding safety shield 60 configured as a Ultimed UtliCare safety device. In one or more embodiments, the embodiment of guiding element 40 illustrated in FIG. 96 may be configured to accommodate a wide range of vial 70 sizes (such as between about 1 mL to at least 10 mL vials 70 and even more milliliters). In one or more embodiments, the embodiment of guiding element 40 illustrated in FIG. 96 may be configured to be used by various professional healthcare providers (such as doctors, nurses, emergency medical technicians, paramedics, midlevel practitioners like nurse practitioners and physician assistants, veterinarians, etc.) and in various settings and situations (such as in emergency medical services, hospitals, doctors' offices, clinics, labs, public health offices, veterinarian offices, homes, adverse working conditions, etc.). In one or more embodiments, the embodiment of guiding element 40 illustrated in FIG. 96 may be used for various medications, such as steroid injections, single or multidose vaccinations, vitamin B12 shots, supplements, medications, epinephrine, antihistamines, antibiotics, etc.

FIG. 97 shows a bottom perspective view of the embodiment of guiding element 40 illustrated in FIG. 96. FIG. 98 shows a right side view, a left side view, a front view, and a rear view of the embodiment of the guiding element illustrated in FIG. 96. FIG. 99 shows a bottom view of the embodiment of guiding element 40 illustrated in FIG. 96. FIG. 100 shows a top view of the embodiment of guiding element 40 illustrated in FIG. 96.

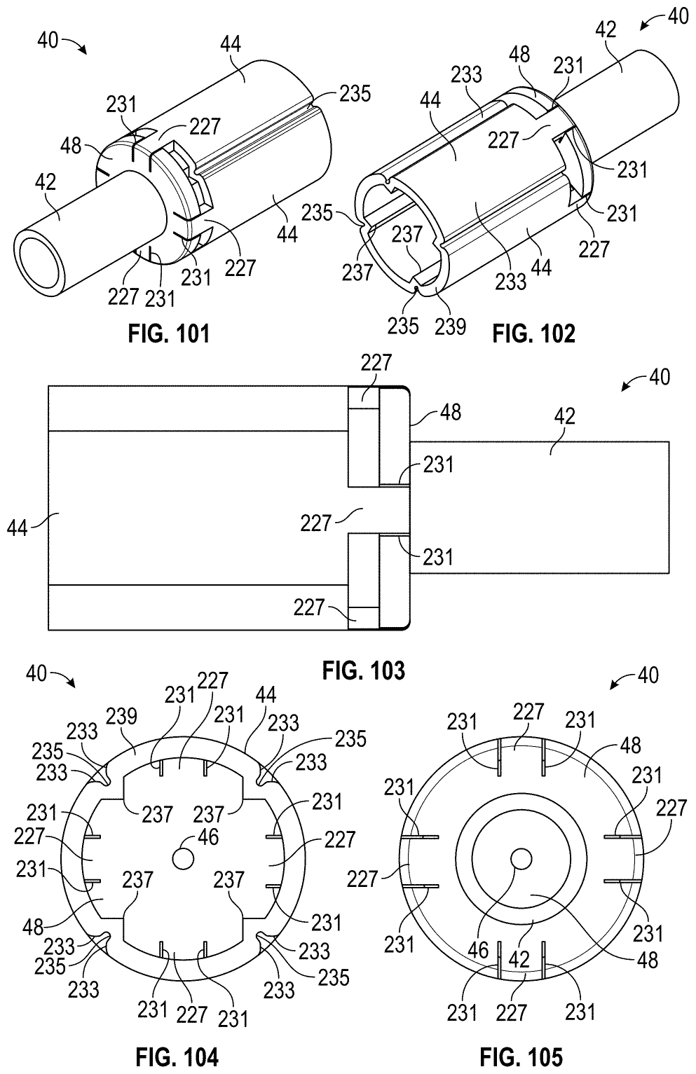

FIG. 101 shows a top perspective view of an alternative embodiment of guiding element 40 configured to receive a syringe 20 that includes a sliding safety shield 60 configured as a Ultimed UtliCare safety device. Although the size of the devices drawing in FIGS. 96-100 and 101-105 may not necessarily reflect such, in one or more embodiments, the diameter of the shaft 42 of the guiding element 40 illustrated in FIG. 101 may be substantially the same size as the diameter of the shaft 42 of the guiding element 40 illustrated in FIG. 96. In one or more embodiments, the diameter of the barrel 44 of the guiding element 40 illustrated in FIG. 101 may be smaller than the diameter of the barrel 44 of the guiding element 40 illustrated in FIG. 96, and the depth at which the recessed portions 233 recess inwardly may be shallower. In one or more embodiments, a smaller barrel 44 may be configured for a single medication use. For example, in one or more embodiments, people who inject epinephrine or daily doses of vitamin B12 into themselves, likely do not need a guiding element 40 that can fit a wide range of vial 70 sizes. Generally speaking, these individuals repeatedly use a 1 mL vial 70 of epinephrine or consistent 5 or 10 mL vials 70 of vitamin B12. As such, these individuals may only need a guiding element 40 with a barrel 44 that can house a consistently-sized vial 70. In one or more embodiments, the smaller, more rigid configuration of the guiding element 40 illustrated in FIG. 101 may be designed to house such 1 mL vials 70.

FIG. 102 shows a bottom perspective view of the embodiment of guiding element 40 illustrated in FIG. 101. FIG. 103 shows a right side view, a left side view, a front view, and a rear view of the embodiment of the guiding element illustrated in FIG. 101. FIG. 104 shows a bottom view of the embodiment of guiding element 40 illustrated in FIG. 101. As shown in FIG. 104 (and FIG. 109), in one or more embodiments, the length at which the recessed portions 233 recess inwardly may be short (that is, shorter in length when compared to the length of the recessed portions 233 of the guiding elements 40 illustrated in FIGS. 96-100, 111-115 and elsewhere). In one or more embodiments, the shortness of the recessed portions 233 may provide barrel 44 with less flexibility. FIG. 105 shows a top view of the embodiment of guiding element 40 illustrated in FIG. 101.

FIG. 106 shows a top perspective view of an alternative embodiment of guiding element 40 configured to receive a syringe 20 that includes a sliding safety shield 60 configured as a Ultimed UtliCare safety device. In one or more embodiments, the embodiment of guiding element 40 illustrated in FIG. 106 may include a shaft 42 that has substantially the same diameter as the shaft 42 of the guiding element 40 illustrated in FIG. 101; however, to accommodate longer needles 30, in one or more embodiments, the length of the shaft 42 of the guiding element 40 illustrated in FIG. 106 may be longer than the shaft 42 of the guiding element 40 illustrated in FIG. 101. To accommodate shorter vials 70, in one or more embodiments, the length of the barrel 44 of the guiding element 40 illustrated in FIG. 106 may be shorter than the barrel 44 of the guiding element 40 illustrated in FIG. 101.

FIG. 107 shows a bottom perspective view of the embodiment of guiding element 40 illustrated in FIG. 106. FIG. 108 shows a right side view, a left side view, a front view, and a rear view of the embodiment of the guiding element illustrated in FIG. 106. FIG. 109 shows a bottom view of the embodiment of guiding element 40 illustrated in FIG. 106. As shown in FIG. 109, in one or more embodiments, the channels 235 and recessed portions 233 of the barrel 44 may assume sharp angles (as opposed to the rounded angles associated with the channels 235 and recessed portions 233 of the guiding elements 40 illustrated in FIGS. 96-105, 111-115 and elsewhere). In one or more embodiments, the sharpness of said angles may provide barrel 44 with less flexibility. FIG. 110 shows a top view of the embodiment of guiding element 40 illustrated in FIG. 106.

FIG. 111 shows a top perspective view of one embodiment of guiding element 40 configured to operably connect with the hub 32 or wings 33 of a needle 30. In one or more embodiments, said needle 30 may be configured to operably connect to a syringe tip 24 configured as a Luer-Lok syringe tip, such as shown in FIG. 1 (or said needle 30 may be configured to operably connect to a safety shield 60 that is configured to operably connect to a syringe tip 24 configured as a Luer-Lok syringe tip, such as shown in FIGS. 2 and 3). In one or more embodiments, said needle 30 may be configured to operably connect to alternative syringe tips 24 or safety shields 60.

In one or more embodiments, the barrel 44 of the guiding element 40 illustrated in FIG. 111 may be substantially the same as the barrel 44 of the guiding element 40 illustrated in FIG. 96. However, in one or more embodiments, to receive and engage the hub 32 or wings 33 of a needle 30, the diameter and length of the shaft 42 of the guiding element 40 illustrated in FIG. 111 may be smaller than the diameter and length of the shaft 42 of the guiding element 40 illustrated in FIG. 96. Although not shown in FIG. 111, in one or more embodiments, the shaft 42 may include a protrusion 43 positioned on or circumference the inner wall of shaft 42, which when engaged by wings 33 of hub 32, may limit the distance needle 30 may travel into shaft 42.

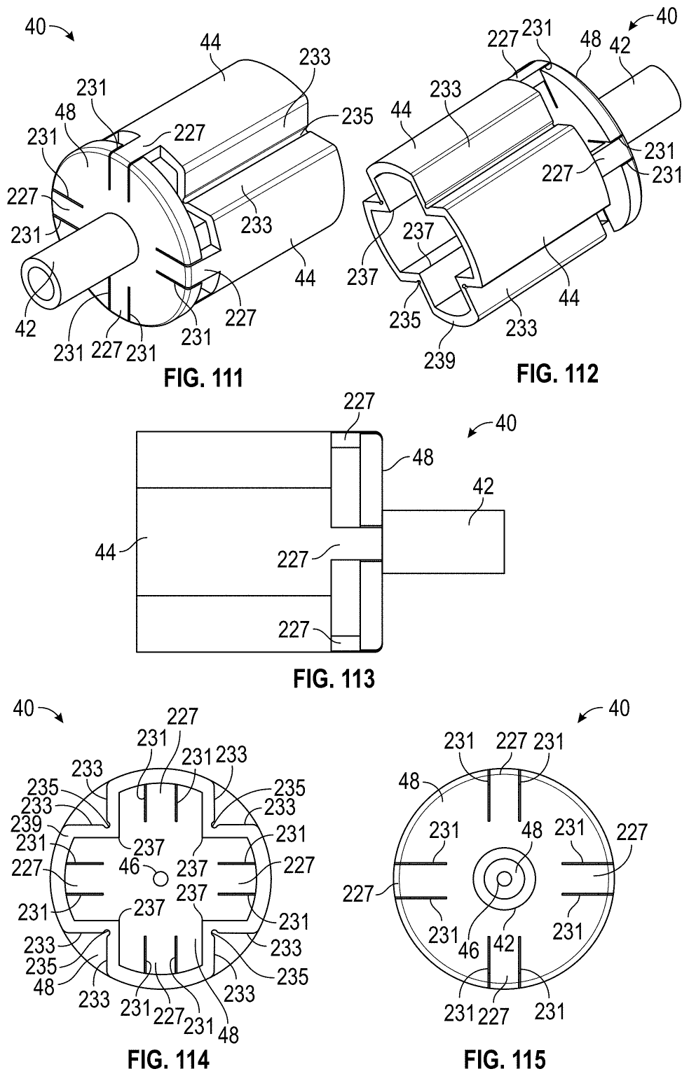

FIG. 112 shows a bottom perspective view of the embodiment of guiding element 40 illustrated in FIG. 111. FIG. 113 shows a right side view, a left side view, a front view, and a rear view of the embodiment of the guiding element illustrated in FIG. 111. FIG. 114 shows a bottom view of the embodiment of guiding element 40 illustrated in FIG. 111. FIG. 115 shows a top view of the embodiment of guiding element 40 illustrated in FIG. 111.

The various components and characteristics of the embodiments of guiding element 40 shown and described in relation to FIGS. 1-115 may, in one or more embodiments, be mixed and matched with other each. For example, in one or more embodiments, the various configurations of channels 235 and recessed portions 233 contained herein may be mixed and matched with the various configurations of shafts 42 and barrels 44, depending on the need. In one or more embodiments, the exact dimensions and configurations of said components may be determined by the versatility needed or not needed. In one or more embodiments, the various embodiments of guiding element 40 shown and described in relation to FIGS. 1-115 may be mixed and matched and used in conjunction with the various embodiments of volume limiter 50 shown and described in relation to FIGS. 1-38 and 116-183 as part of various embodiments of medication delivery device 10. In one or more embodiments, the various embodiments of guiding element 40 and volume limiter 50 shown and described herein, maybe configured to be operably connected to the various embodiments of syringe 20 shown and described herein. The FIGS. 96-115 are not necessarily to scale in relation to each other. In one or more embodiments, guiding element 40 may be configured to operably connect to syringes 20 or needles 30 made by various manufactures. In one or more embodiments, guiding element 40 may be configured to engage syringes 20 or needles 30 made of plastic, glass and/or stainless steel.

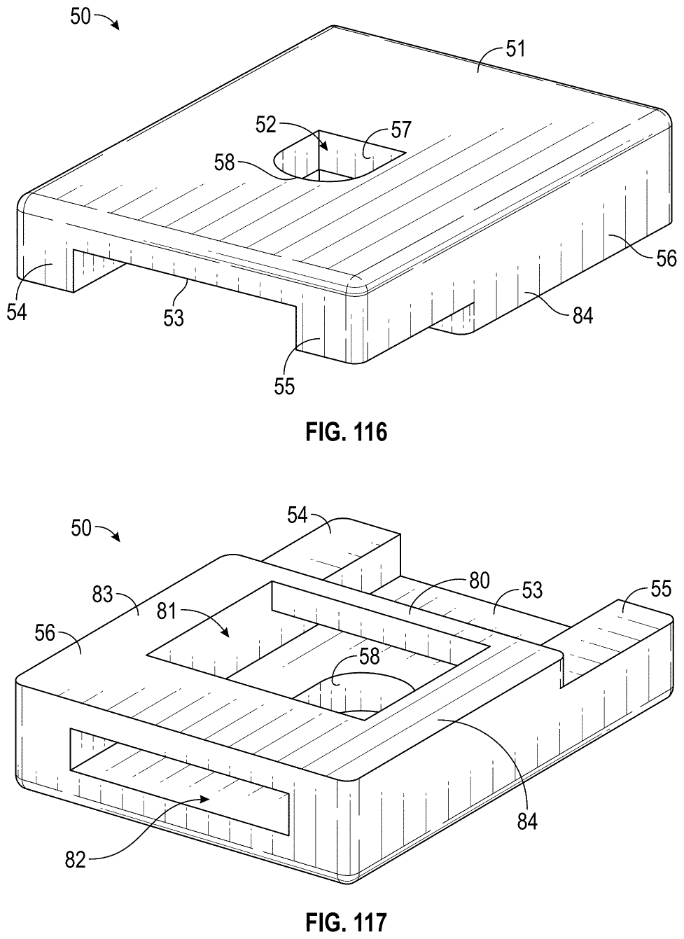

FIGS. 116-183 show embodiments of volume limiter 50 configured for at least the purpose of operably connecting to syringes 20 and limiting the amount of medication those syringes 20 can draw from vials 70, thereby limiting the amount of medication that can be administered to a patient, preventing overdosing, and ensuring that the correct dosage is administered. In one or more embodiments, the embodiments of volume limiter 50 illustrated in FIGS. 116-183 may be configured to operably connect to syringes 20 of various sizes and configurations. In one or more embodiments, the embodiments of volume limiter 50 illustrated in FIGS. 116-183 may include various configurations of openings 81 and 82 in order to receive syringes 20 of various size and configurations. In one or more embodiments, the embodiments of volume limiter 50 illustrated in FIGS. 116-183 may include various configurations of opening 52 in order to receive various plunger 25 size and configurations (such as cruciform plungers 25). In one or more embodiments, the various embodiments of volume limiter 50 illustrated in FIGS. 116-183 may be used in conjunction with medication delivery devices 10 capable of being operably connected to various embodiments of guiding element 40 that are capable of housing vials 70 of different sizes, guiding needles 30 into substantially the center of those vials 70, and securing or otherwise engaging those vials 70 while a user draws medication from them.

FIG. 116 shows a top perspective view of one embodiment of volume limiter 50. In one or more embodiments, the volume limiter 50 shown in FIG. 116 is configured to be operably connected to a syringe 20 configured as a BD 1 mL syringe. In one or more embodiments, as shown in FIG. 116, volume limiter 50 may include top surface 51, opening 52 through which plunger 25 may be inserted, bottom surface 53, first bottom ledge 54, second bottom ledge 55, first flange housing element 56, and retention member 80 (not fully shown in FIG. 116). In one or more embodiments, opening 52 may include first surface 57 (which may assume the contour of the engagement section 26 of plunger 25) and second surface 58 (which may assume an alternative contour). As shown in FIG. 116, in one or more embodiments, first surface 57 may be configured to be flat in order to conform to a notched or rebated engagement section 26 and second surface 58 may be configured to be rounded.

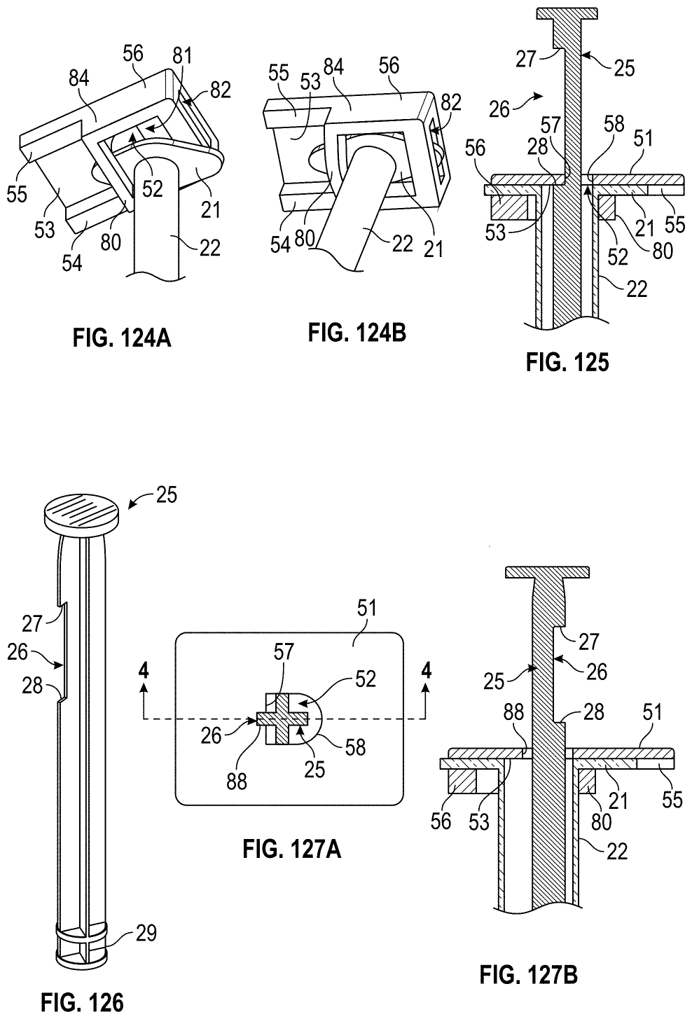

FIG. 117 shows a bottom perspective view of the embodiment of volume limiter 50 illustrated in FIG. 116. In one or more embodiments, first flange housing element 56 may include a first flange housing arm 83 and a second flange housing arm 84. In one or more embodiments, the first flange housing arm 83 may be integral to the first bottom ledge 54 and the second flange housing arm 84 may be integral to the second bottom ledge 55. In one or more embodiments, the retention member 80 may be an integral part of first flange housing element 56 and may extend from and be located between first flange housing arm 83 and second flange housing arm 84. In one or more embodiments, an opening 81 may be located under the bottom surface 53 of volume limiter 50. In one or more embodiments, opening 81 may be located in between first flange housing arm 83, second flange housing arm 84, and retention member 80 such that a flange 21 of syringe 20 may be inserted into opening 81. In one or more embodiments, an opening 82 may be located in the rear or back of flange housing element 56 such that opening 82 hollows out first flange housing element 56 and continues over retention member 80 such that a flange 21 may be extended into or beyond opening 82 (see for example FIGS. 124B, 125 and 127B). Such configuration may allow volume limiter 50 to be operably connected to syringes 20 with flanges 21 of various sizes and configurations. In one or more embodiments, the cavity of opening 81 may lead into opening 82. In one or more embodiments, first flange housing arm 83 and second flange housing arm 84 may be configured to allow flange 21 to slide in between the two arms. In one or more embodiments, first bottom ledge 54 and second bottom ledge 55 may be configured to allow flange 21 to slide in between the two ledges and be secured by the same. In one or more embodiments, first flange housing element 56 may be configured to allow flange 21 to slide into and be secured by it.

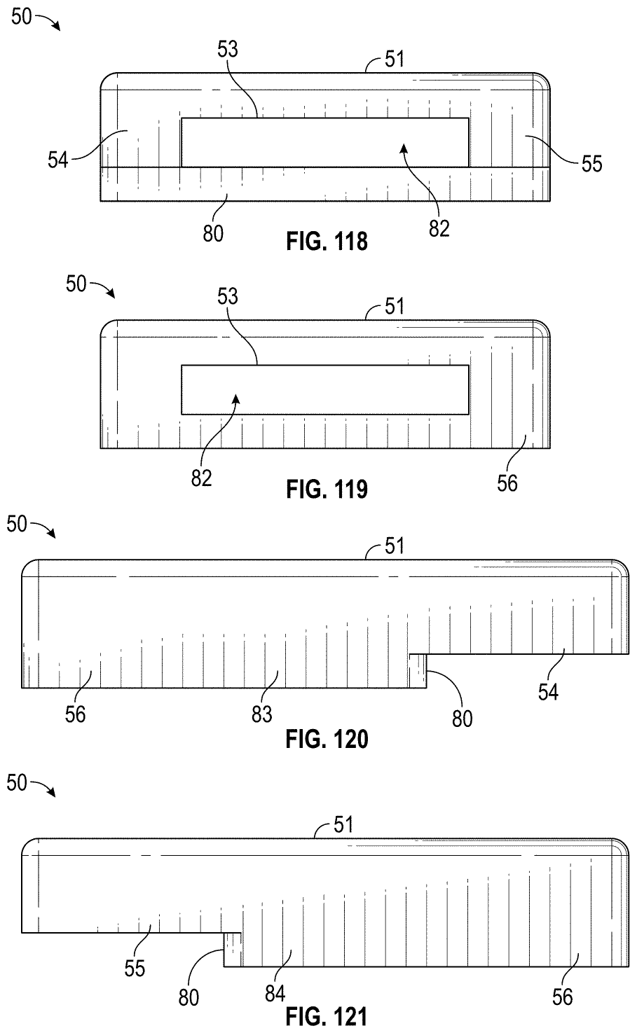

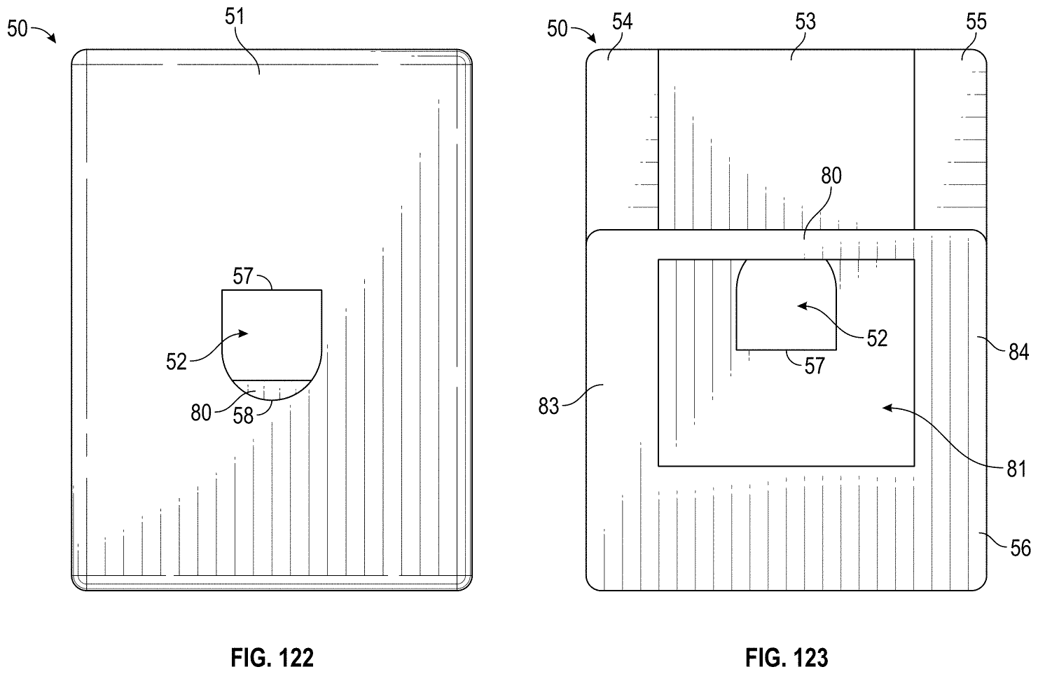

FIG. 118 shows a front view of the embodiment of volume limiter 50 illustrated in FIG. 116. FIG. 119 shows a rear view of the embodiment of volume limiter 50 illustrated in FIG. 116. FIG. 120 shows a right side view of the embodiment of volume limiter 50 illustrated in FIG. 116. FIG. 121 shows a left side view of the embodiment of volume limiter 50 illustrated in FIG. 116. FIG. 122 shows a top view of the embodiment of volume limiter 50 illustrated in FIG. 116. FIG. 123 shows a bottom view of the embodiment of volume limiter 50 illustrated in FIG. 116.

FIGS. 124A-124B show how one embodiment of syringe 20 may be operably connected to one embodiment of volume limiter 50.

FIG. 124A shows a bottom perspective view of one embodiment of volume limiter 50 with the flange 21 of syringe 20 inserted into opening 81. In one or more embodiments, volume limiter 50 may be operably connected to syringe 20 by inserting one end of flange 21 into opening 81 such that flange 21 travels between first flange housing arm 83 and second flange housing arm 84 and into opening 82 between first bottom ledge 54 and second bottom ledge 55 such that barrel 22 of syringe 20 engages and stretches retention member 80 sufficient to create enough space to insert the other end of flange 21 into opening 81 and opening 82.

FIG. 124B shows an alternative bottom perspective view of the embodiment of volume limiter 50 illustrated in FIG. 124A with the flange 21 of syringe 20 fully inserted into first flange housing element 56, and with syringe 20 being resiliently engaged by retention member 80. As shown in FIG. 124B, in one or more embodiments, retention member 80 may resiliently stretch or bend around syringe 20. Such configuration may be a means for resiliently securing volume limiter 50 to syringe 20.

Although not shown in FIGS. 124A-124B, in one or more embodiments, other embodiments of volume limiter 50 contained herein, such as the embodiments of volume limiter 50 illustrated and described in relation to FIGS. 116-123, 125-183, may be configured to operably connect to syringes 20 as described and shown in relation to FIGS. 124A-124B.

FIGS. 125-127B show how various embodiments of plunger 25 may be inserted, engaged, disengaged and withdrawn from various embodiments of volume limiter 50. In one or more embodiments, the structures and functionalities of the volume limiters 50 and the processes of inserting, engaging, disengaging and withdrawing of the plungers 25 as shown and described in relation to FIG. 125-127B (as well as the other volume limiters 50 and plungers 25 shown and described in relation to FIGS. 116-183) may include the structures and functionalities of the volume limiter 50 and the processes of inserting, engaging, disengaging and withdrawing of the plunger 25 as shown and described in relation to FIGS. 12A-12H (which structures, functionalities and processes of said embodiments are hereby incorporated herein and applied to the volume limiters 50 and plungers 25 shown and described in relation to FIGS. 116-183 by reference). For example, in one or more embodiments, medication delivery device 10 may provide a means for limiting the amount of medication that can be drawn from vial 70, thereby limiting the amount of medication that can be administered to a patient. For example, once volume limiter 50 has been operably connected to syringe 20 and plunger 25 has been inserted through opening 52 with the engagement section 26 aligned with the first surface 57 of opening 52 (such as shown in FIG. 125), in one or more embodiments, plunger 25 may travel within syringe barrel 22 until first end 27 of plunger 25 engages the top surface 51 of volume limiter 50. In one or more embodiments, if the engagement section 26 is not aligned with the first surface 57 of opening 52, plunger 25 may travel within syringe barrel 22 until plunger stopper 29 hits the bottom of syringe barrel 22. Either way, in one or more embodiments, once first end 27 of plunger 25 engages the top surface 51 of volume limiter 50 or once plunger stopper 29 hits the bottom of syringe barrel 22, a user may be prevented from expelling substances (including liquid and air) from syringe 20 in a particular depression. Such configuration may be at least one way medication delivery device 10 may provide for limiting the amount of medication that can be injected into a patient, limiting the amount of air a user may force into vial 70, preventing overdosing, and ensuring that the correct dosage is administered. In one or more embodiments, medication delivery device 10 may provide a means for volume limiter 50 to automatically limit the amount of medication that can be drawn from vial 70, thereby limiting the amount of medication that can be administered to a patient. For example, in one or more embodiments, once volume limiter 50 has been operably connected to syringe 20, with syringe 20 being resiliently secured by retention member 80 (such as shown and described in relation to FIGS. 124A-124B), and plunger 25 has been inserted into the opening 52 and depressed with the engagement section 26 aligned with the first surface 57 of opening 52 (such as shown in FIG. 125), in one or more embodiments, the first surface 57 may automatically adjust to the contour of plunger 25 and engage the engagement section 26 because of retention member's 80 constant force and resilient securement of syringe 20. In one or more embodiments, once second end 28 travels through opening 52 and passes the bottom surface 53, retention member's 80 constant force and resilient securement may cause first surface 57 of opening 52 to automatically snap against and apply force onto engagement section 26, and thereby limit the distance plunger 25 may travel to the distance between first end 27 and second end 28. Such means for automatically engaging volume limiter 50 may, in one or more embodiments, allow plunger 25 to be provided to a user in a drawn position (with second end 28 positioned against or proximal to bottom surface 53 and first surface 57 engaging the engagement section 26). In one or more embodiments, providing a plunger 25 in said drawn position, may make medication delivery device 10 ready for the user to: (1) insert an attached needle 30 into vial 70 with the aid of guiding element 40, (2) depress plunger 25 and force air into vial 70, and (3) let pressure force plunger 25 back and fill syringe 20 with medication from vial 70 up to the point at which second end 28 engages bottom surface 53. Such configuration may limit the amount of medication that can be drawn from vial 70 and thereby limit the amount of medication that can be administered to a patient, prevent overdosing, and ensure that the correct dosage is administered.

FIG. 125 shows a cross sectional view of one embodiment of volume limiter 50 with plunger 25 inserted through opening 52 and syringe 20 fully inserted into the first flange housing element 56, and with syringe 20 being engaged by retention member 80. In one or more embodiments, the volume limiter 50 shown in FIG. 125 may be similar to the embodiment of the volume limiter 50 illustrated in FIG. 12B such that the sectional view in FIG. 125 may, generally speaking, follow along lines 1-1 shown in FIG. 12B. As shown in FIG. 125, in one or more embodiments, if the engagement section 26 is aligned with the first surface 57 of opening 52, plunger 25 may travel within syringe barrel 22 until second end 28 of plunger 25 engages the bottom surface 53 of volume limiter 50. Such configuration may be at least one way medication delivery device 10 may provide for limiting the amount of medication that can be drawn into syringe barrel 22 from vial 70 and thereby limiting the amount of medication that can be injected into a patient, preventing overdosing, and ensuring that the correct dosage is administered. Although not shown in FIG. 125, in one or more embodiments, the limiting and automatic engagement functionalities of the embodiments of volume limiter 50 and plunger 25 shown and described in relation to FIG. 125, may also apply and be adapted to embodiments of a cruciform plunger 25 and a volume limiter 50 configured to engage a cruciform plunger 25 (such as shown and described in FIGS. 126, 127A, 127B, 136-151, 160-167, and 168-175).

FIG. 126 shows a perspective view of one embodiment of plunger 25 designed as a cruciform. As shown in FIG. 126, in one or more embodiments, a cruciform plunger 25 may include four longitudinally situated ribs approximately 90 degrees in relation to each other. Like the embodiment of plunger 25 shown and described in relation to FIGS. 11A-11B, in one or more embodiments, the cruciform designed plunger 25 may include an engagement section 26 and a plunger stopper 29. In one or more embodiments, the engagement section 26 of a cruciform plunger 25 may be a notched or rebated section of one of the four longitudinally situated ribs. In one or more embodiments, the engagement section 26 of a cruciform plunger 25 may include a first end 27 at one end and a second end 28 at an alterative end of the engagement section 26.

FIG. 127A shows a top view of one embodiment of a volume limiter 50 and a horizontal cross sectional view of one embodiment of a cruciform plunger 25 inserted through opening 52 in volume limiter 50, with volume limiter 50 including a notch 88 configured to secure the cruciform plunger 25. As shown in FIG. 127A, in one or more embodiments, notch 88 is sized to allow a longitudinally situated rib of a cruciform plunger 25 to fit into and be secured by it.

FIG. 127B shows a cross sectional view along lines 4-4 of the embodiments of volume limiter 50 and cruciform plunger 25 illustrated in FIG. 127A, albeit FIG. 127B also shows a syringe 20 fully inserted into first flange housing element 56 and engaged by retention member 80, with the cruciform plunger 25 having been repositioned and disengaged from the volume limiter's 50 notch 88. In one or more embodiments, cruciform plunger 25 may be inserted into opening 52 with its engagement section 26 aligned or not aligned with the volume limiter's 50 notch 88. If the engagement section 26 of the cruciform plunger 25 is not aligned with notch 88, the cruciform plunger 25 may be twisted such that the rib containing its engagement section 26 becomes aligned with notch 88 such that notch 88 may secure said rib. In one or more embodiments, retention member 80 may provide constant force and resilient securement on syringe 20 which may cause first surface 57 of opening 52 to automatically snap against and apply force onto engagement section 26 when the engagement section 26 is aligned with notch 88, thereby limiting the distance cruciform plunger 25 may travel to the distance between first end 27 and second end 28. Such configuration may limit the amount of medication that can be drawn from vial 70 and thereby limit the amount of medication that can be administered to a patient, prevent overdosing, and ensure that the correct dosage is administered.

In one or more embodiments, when plunger 25 (including a cruciform plunger 25) is twisted or otherwise moved relative to volume limiter 50, volume limiter 50 may resiliently shift positions or slide along flange 21. In one or more embodiments, when the engagement section 26 engaged by first surface 27 or, in the case of a cruciform plunger 25, when it is seated in notch 88, plunger 25 may be twisted to disengage the engagement section 26 from first surface 57 or notch 88, as the case may be, thereby allowing plunger 25 to travel further to check for “flash” or for other reasons. Such configuration may provide a means for disengaging volume limiter 50 from plunger 25 (including a cruciform plunger 25). In one or more embodiments, plunger 25 (including a cruciform plunger 25) may be twisted less than 180 degrees to disengage volume limiter 50 from plunger 25 and allow plunger 25 (including a cruciform plunger 25) to travel further. In one or more embodiments, alternative or additional means may be provided for disengaging volume limiter 50 from plunger 25 (including a cruciform plunger 25) or some other aspect of medication delivery device 10 to allow plunger 25 to travel further. For example, in one or more embodiments, volume limiter 50 may be designed to allow a user to shift or otherwise reposition volume limiter 50 in order to disengage it from plunger 25 (including a cruciform plunger 25) or some other component of medication delivery device 10, thereby allowing plunger 25 to travel further. Such configuration(s) may be in addition to or alternative to being able to twist plunger 25 (including a cruciform plunger 25) to disengage the volume limiter 50. In one or more embodiments, plunger 25 (including a cruciform plunger 25) may be twisted such that engagement section 26 is proximal to second surface 58, thereby disengaging volume limiter 50 from plunger 25 (including a cruciform plunger 25). In one or more embodiments, once plunger 25 (including a cruciform plunger 25) is twisted so that engagement section 26 is not proximal to first surface 57 or notch 88, as the case may be, plunger 25 (including a cruciform plunger 25) may travel within syringe barrel 22 at a greater distance because engagement section 26 does not catch on bottom surface 53 or top surface 51. In one or more embodiments, such an arrangement may allow a user, who has already inserted needle 30 into a patient, to draw back and check for blood or extract substances from a patient. In one or more embodiments, after twisting plunger 25 (including a cruciform plunger 25) or otherwise disengaging volume limiter 50 from engagement section 26, plunger 25 may be removed through opening 52 and separated from medication delivery device 10. In one or more embodiments, twisting plunger 25 (including a cruciform plunger 25) may cause volume limiter 50 to shift (such as substantially perpendicularly to plunger 25) to aid in allowing plunger 25 to travel further. In one or more embodiments, volume limiter 50 may be manually repositioned, shifted or otherwise moved (such as substantially perpendicularly in relation to plunger 25) by a user in order to move first surface 57 or notch 88, as the case may be, away from engagement section 26 and disengage volume limiter 50 from plunger 25 (including a cruciform plunger 25). Such configuration may be one embodiment of a means for disengaging volume limiter 50 from plunger 25 (including a cruciform plunger 25) to allow plunger 25 to travel further and check for blood or other factors.

Although not shown in FIGS. 127A and 127B, in one or more embodiments, the ability to twist and disengage the embodiments of volume limiter 50 and cruciform plunger 25 shown and described in relation to FIGS. 127A and 127B, may also apply and be adapted to embodiments of a non-cruciform plunger 25 and a volume limiter 50 configured to engage a non-cruciform plunger 25 (such as shown and described in FIGS. 116-123, 152-159, and 176-183).

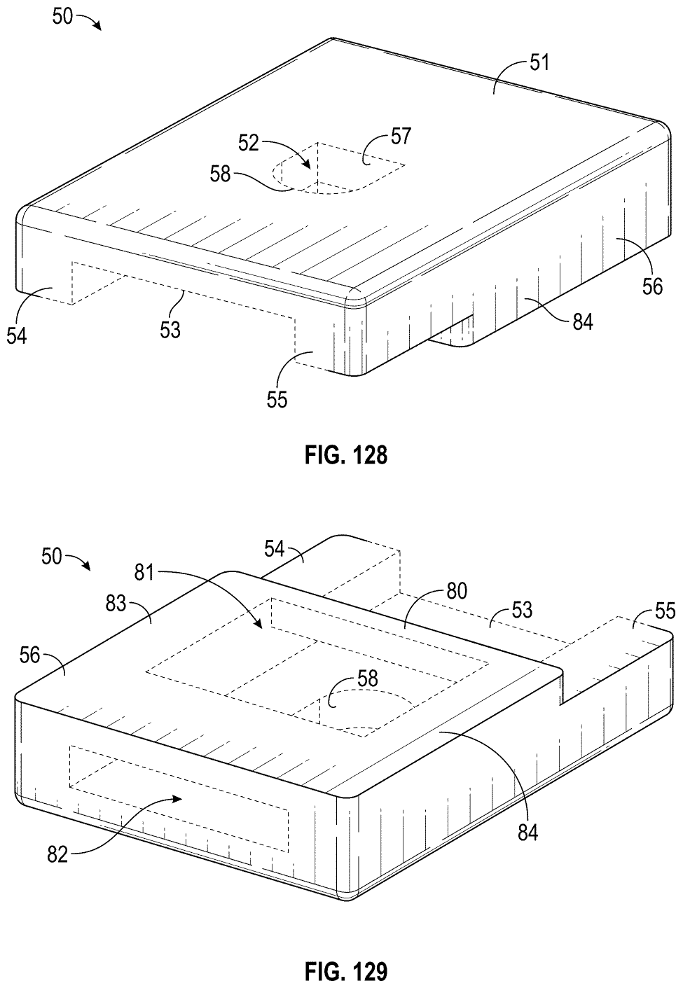

FIGS. 128-135 show that the configuration of components of the embodiment of volume limiter 50 illustrated in FIGS. 116-127 may vary. In one or more embodiments, the possible modifications to volume limiter 50 are not limited to the aspects of volume limiter 50 that are dot lined in FIGS. 128-135.

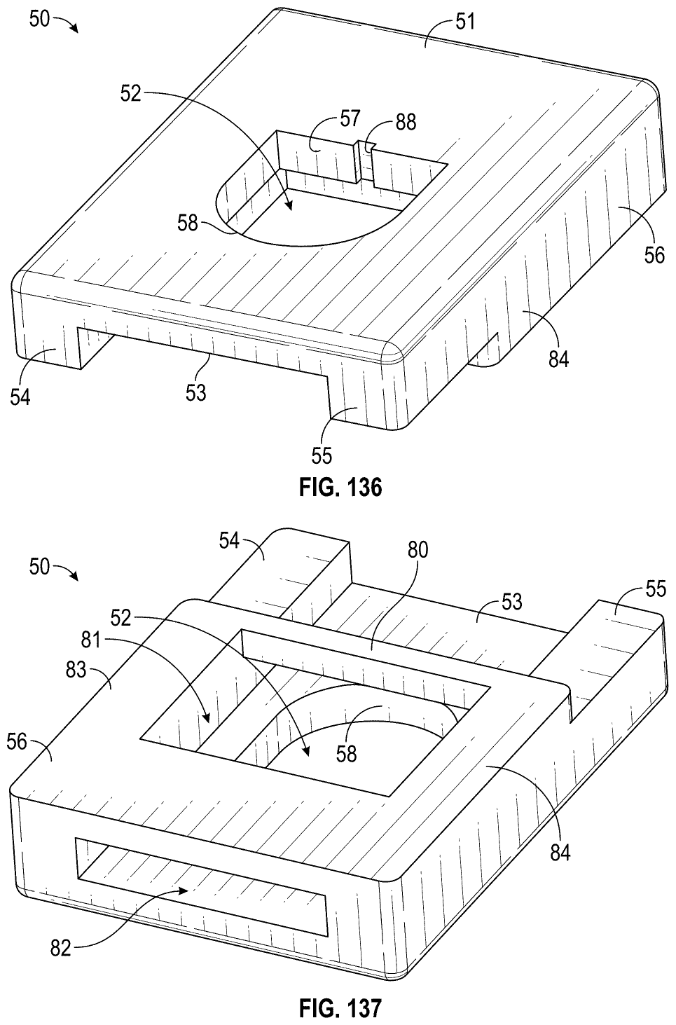

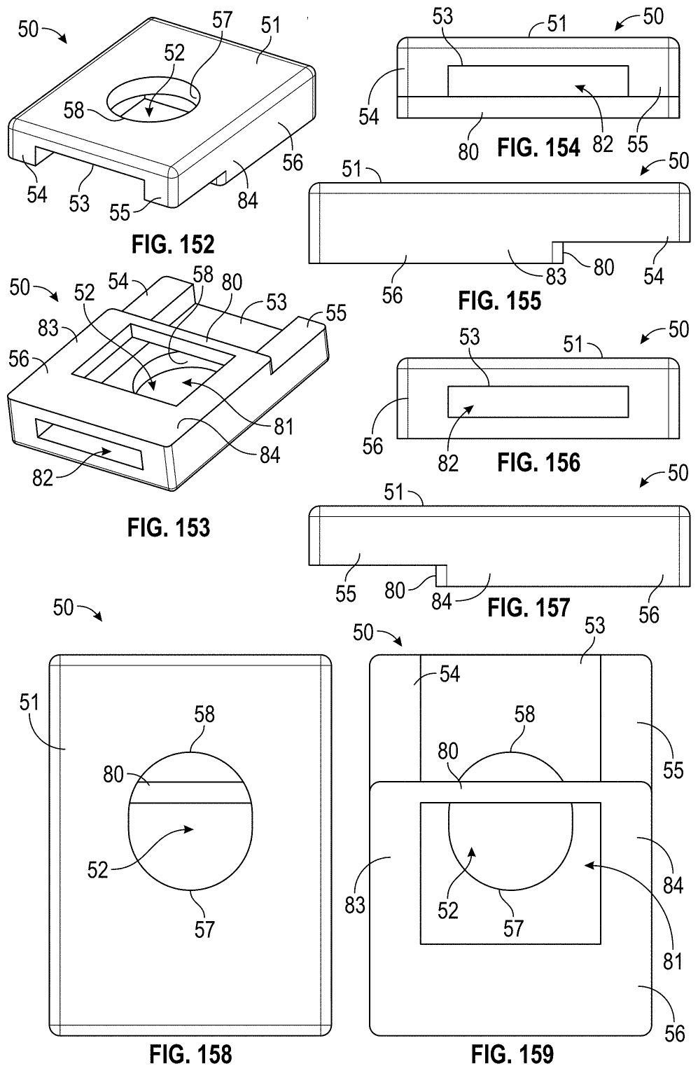

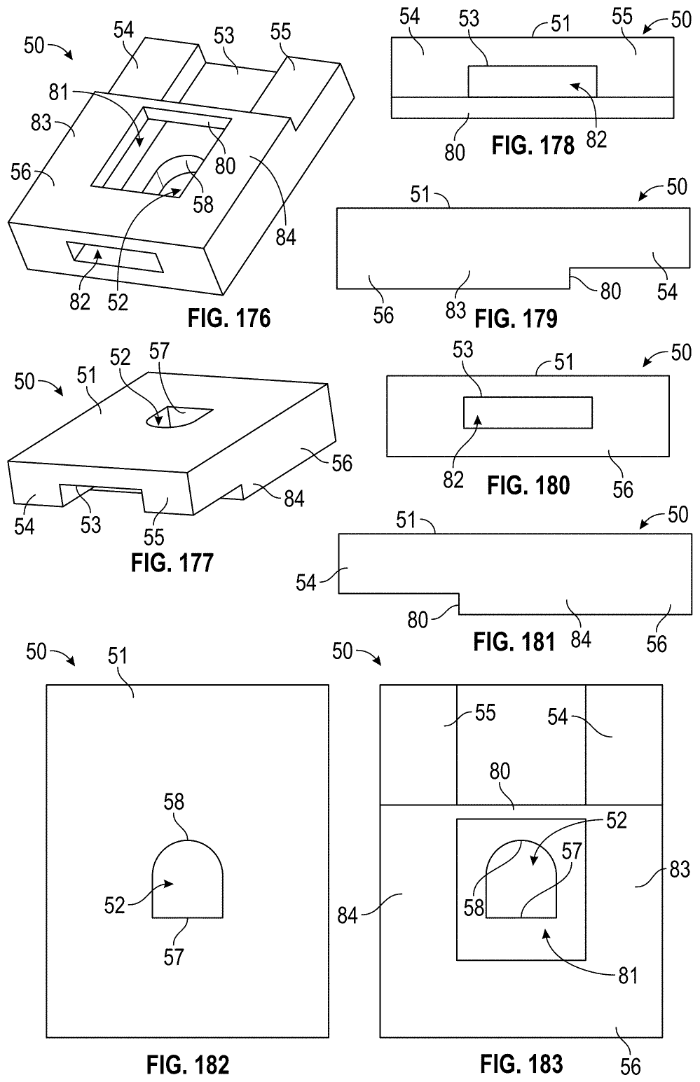

FIG. 128 shows a top perspective view of one embodiment of volume limiter 50 illustrated in FIG. 116 with various components dot lined. As shown by the dotted lines in FIG. 128, in one or more embodiments, the width, circumference, depth, shape, location, and other dimensions and configurations of opening 52 may vary. For example, in one or more embodiments, opening 52 may be rectangle, square, circular, oval, oblong, or some other shape to fit plungers 25 of various sizes and configurations. In one or more embodiments, one part of opening 52 (such as first surface 57) may be one shape (such as flat) and another part of opening 52 (such as second surface 58) may be another shape (such as rounded), such as shown in FIGS. 176-183. In one or more embodiments, first surface 57 may assume the same or substantially similar profile as compared to the profile of second surface 58 (such as shown in FIGS. 152-159) such that opening 52 is a single shape. Although not shown in FIG. 128, in one or more embodiments, opening 52 may include any number of notches (such as notch 88 as shown in FIGS. 136-143), indentations, or other cuts into first surface 57 and/or second surface 58 in order to better engage or correspond to the shape of various plungers 25 (such as a cruciform plunger 25) or for other reasons. As shown by the dotted lines in FIG. 128, in one or more embodiments, the thickness, and other dimensions and configurations of volume limiter's top surface 51 and bottom surface 53 may vary. As shown by the dotted lines in FIG. 128, in one or more embodiments, the width, and other dimensions and configurations of volume limiter's first bottom ledge 54 and second bottom ledge 55 may vary to fit flanges 21 of various sizes and configurations. Although not shown in FIG. 128, in one or more embodiments, the width, length, shape, and other dimensions and configurations of volume limiter's top surface 51 and bottom surface 53 may vary. Although not shown in FIG. 128, in one or more embodiments, the height, length, location, and other dimensions and configurations of volume limiter's first bottom ledge 54 and second bottom ledge 55 may vary to fit flanges 21 of various sizes and configurations.

FIG. 129 shows a bottom perspective view of the embodiment of volume limiter 50 illustrated in FIG. 128 with various components dot lined. As shown by the dotted lines in FIG. 129, in one or more embodiments, the width, circumference, depth, shape, location, and other dimensions and configurations of opening 81 and opening 82 may vary. For example, in one or more embodiments, opening 81 and opening 82 may be rectangle, square, circular, oval, oblong, or some other shape to fit flanges 21 and syringes 20 of various sizes and configurations. As shown by the dotted lines in FIG. 129, in one or more embodiments, the height, and other dimensions and configurations of retention member 80 may vary to adjust its rigidity or flexibility and correlating tension as needed. Although not shown in FIG. 129, in one or more embodiments, the width, length, shape, location, and other dimensions and configurations of retention member 80 may vary to adjust its rigidity or flexibility and correlating tension as needed. As shown by the dotted lines in FIG. 129, in one or more embodiments, the width, and other dimensions and configurations of first flange housing element 56, first flange housing arm 83 and second flange housing arm 84 may vary in order to receive flanges 21 and syringes 20 of various sizes and configurations. Although not shown in FIG. 129, in one or more embodiments, the height, shape, location, and other dimensions and configurations of first flange housing element 56, first flange housing arm 83 and second flange housing arm 84 may vary in order to receive flanges 21 and syringes 20 of various sizes and configurations.

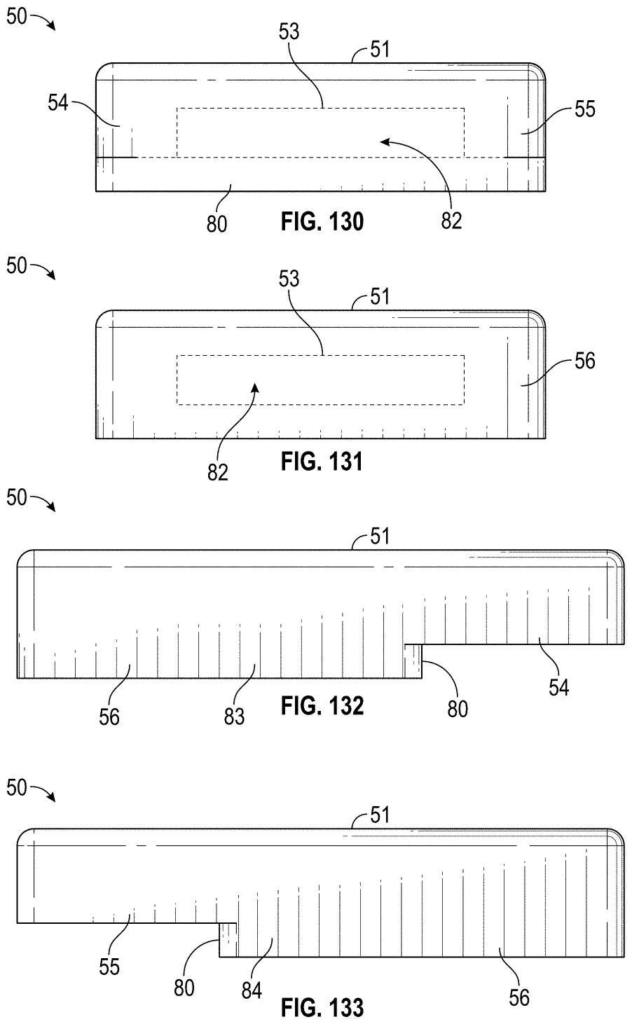

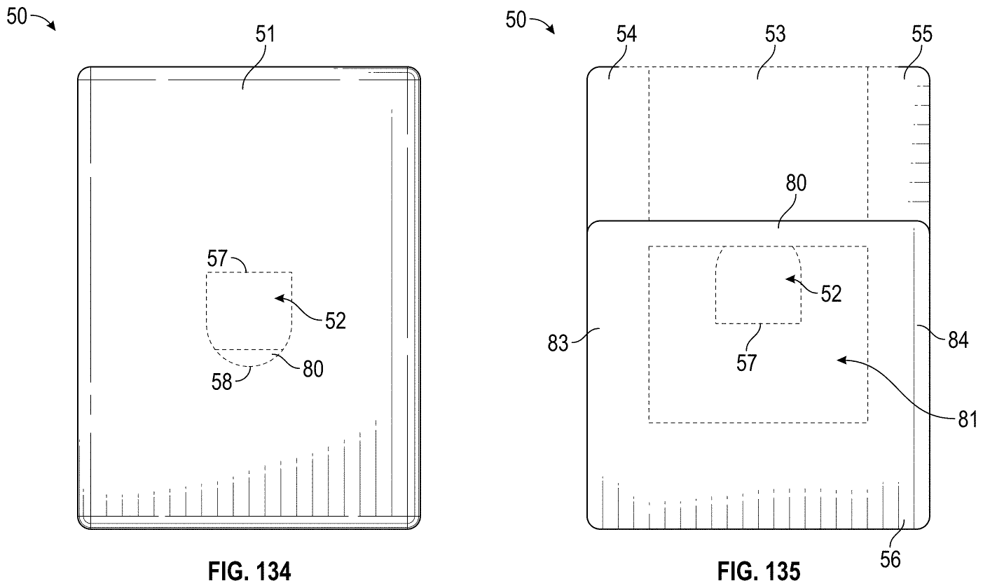

FIG. 130 shows a front view of the embodiment of volume limiter 50 illustrated in FIG. 128. FIG. 131 shows a rear view of the embodiment of volume limiter 50 illustrated in FIG. 128. As shown by the dotted lines in FIG. 131, in one or more embodiments, volume limiter 50 may not include an opening 82 that extends all the way through first flange housing element 56 (such as shown in FIGS. 10A-10E). FIG. 132 shows a right side view of the embodiment of volume limiter 50 illustrated in FIG. 128. FIG. 133 shows a left side view of the embodiment of volume limiter 50 illustrated in FIG. 128. FIG. 134 shows a top view of the embodiment of volume limiter 50 illustrated in FIG. 128. FIG. 135 shows a bottom view of the embodiment of volume limiter 50 illustrated in FIG. 128. Although not shown in FIGS. 128-135, in one or more embodiments, volume limiter 50 may be configured with various protrusions, ridges, indentations, or other configurations or materials, or combinations thereof, which may aid in securing syringe 20 to volume limiter 50. In one or more embodiments, volume limiter's 50 securement to syringe 20 may be temporary, permanent, releasable, adjustable, resilient or otherwise, or any combination thereof.

FIGS. 136-151 show alternative embodiments of volume limiter 50 configured for at least the purpose of being operably connected to syringes 20 with cruciform plungers 25.

FIG. 136 shows a top perspective view of an alternative embodiment of volume limiter 50 that includes a notch 88. In one or more embodiments, the volume limiter 50 shown in FIG. 136 is configured to be operably connected to a syringe 20 configured as a BD 3 mL syringe with a cruciform plunger 25. In one or more embodiments, as shown in FIG. 136, volume limiter 50 may include top surface 51, opening 52 through which plunger 25 may be inserted, bottom surface 53, first bottom ledge 54, second bottom ledge 55, first flange housing element 56, and retention member 80 (not fully shown in FIG. 136). In one or more embodiments, opening 52 may include first surface 57 (which may assume the contour of the engagement section 26 of plunger 25) and second surface 58 (which may assume an alternative contour). As shown in FIG. 136, in one or more embodiments, first surface 57 may include notch 88 in order to engage the engagement section 26 of a cruciform plunger 25 and second surface 58 may be configured to be rounded.

FIG. 137 shows a bottom perspective view of the embodiment of volume limiter 50 illustrated in FIG. 136. In one or more embodiments, first flange housing element 56 may include first flange housing arm 83 and second flange housing arm 84. In one or more embodiments, the first flange housing arm 83 may be integral to the first bottom ledge 54 and the second flange housing arm 84 may be integral to the second bottom ledge 55. In one or more embodiments, the retention member 80 may be an integral part of first flange housing element 56 and may extend from and be located between first flange housing arm 83 and second flange housing arm 84. In one or more embodiments, opening 81 may be located under the bottom surface 53 of volume limiter 50. In one or more embodiments, opening 81 may be located in between first flange housing arm 83, second flange housing arm 84, and retention member 80 such that flange 21 of syringe 20 may be inserted into opening 81. In one or more embodiments, an opening 82 may be located in the rear or back of flange housing element 56 such that opening 82 hollows out first flange housing element 56 and continues over retention member 80 such that flange 21 may be extended into or beyond opening 82 (see for example FIGS. 124B, 125 and 127B). Such configuration may allow volume limiter 50 to be operably connected to syringes 20 with flanges 21 of various sizes and configurations. In one or more embodiments, the cavity of opening 81 may lead into opening 82. In one or more embodiments, first flange housing arm 83 and second flange housing arm 84 may be configured to allow flange 21 to slide in between the two arms. In one or more embodiments, first bottom ledge 54 and second bottom ledge 55 may be configured to allow flange 21 to slide in between the two ledges and be secured by the same. In one or more embodiments, first flange housing element 56 may be configured to allow flange 21 to slide into and be secured by it.

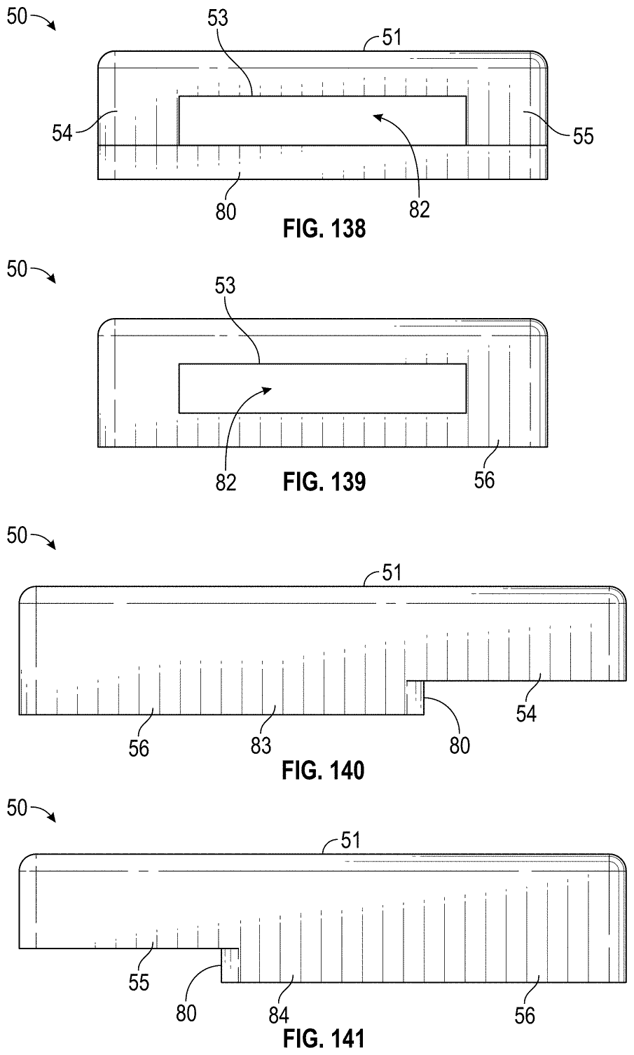

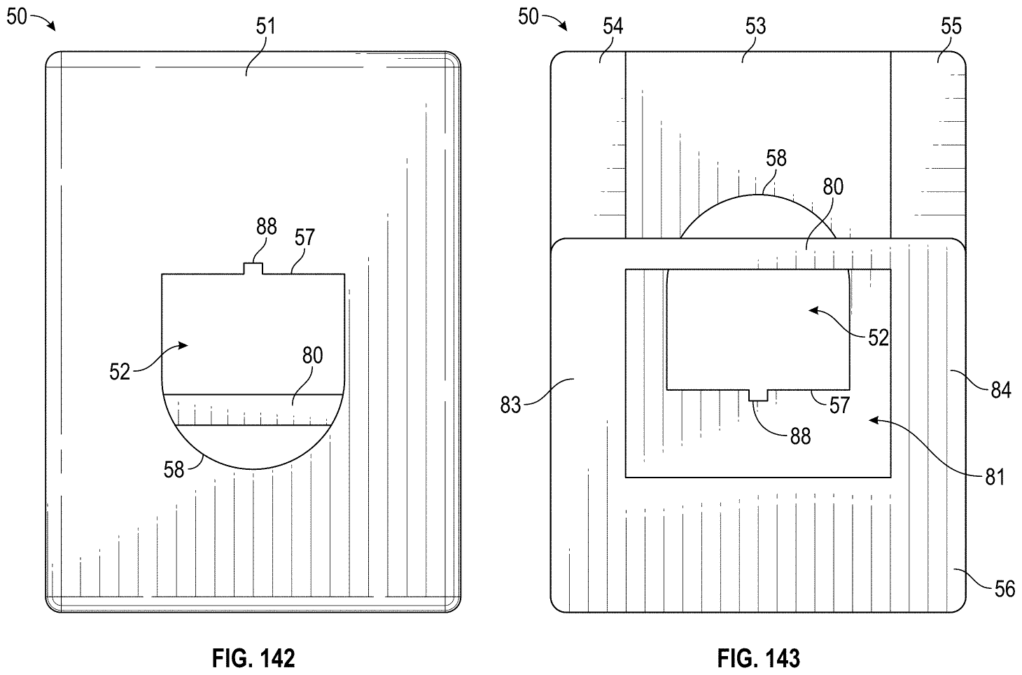

FIG. 138 shows a front view of the embodiment of volume limiter 50 illustrated in FIG. 136. FIG. 139 shows a rear view of the embodiment of volume limiter 50 illustrated in FIG. 136. FIG. 140 shows a right side view of the embodiment of volume limiter 50 illustrated in FIG. 136. FIG. 141 shows a left side view of the embodiment of volume limiter 50 illustrated in FIG. 136. FIG. 142 shows a top view of the embodiment of volume limiter 50 illustrated in FIG. 136. FIG. 143 shows a bottom view of the embodiment of volume limiter 50 illustrated in FIG. 136.

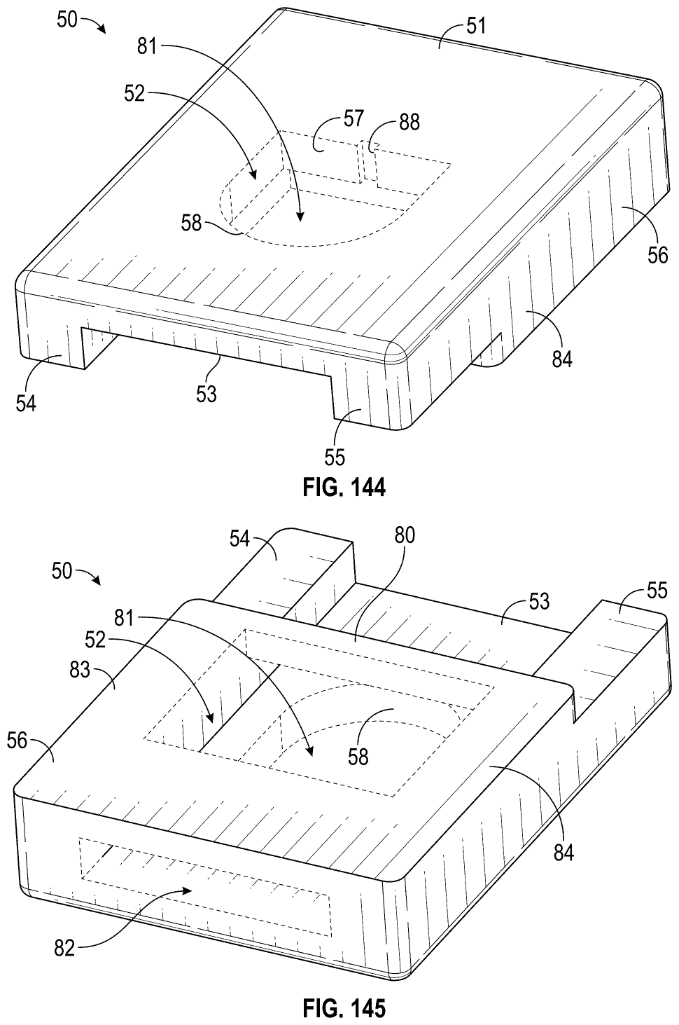

FIGS. 144-151 show that the configuration of components of the embodiment of volume limiter 50 illustrated in FIGS. 136-143 may vary. In one or more embodiments, the possible modifications to volume limiter 50 are not limited to the aspects of volume limiter 50 that are dot lined in FIGS. 144-151.

FIG. 144 shows a top perspective view of one embodiment of volume limiter 50 illustrated in FIG. 136 with various components dot lined. As shown by the dotted lines in FIG. 144, in one or more embodiments, the width, circumference, depth, shape, location, and other dimensions and configurations of opening 52 may vary. For example, in one or more embodiments, opening 52 may be rectangle, square, circular, oval, oblong, or some other shape to fit plungers 25 of various sizes and configurations. In one or more embodiments, one part of opening 52 (such as first surface 57) may be one shape (such as flat) and another part of opening 52 (such as second surface 58) may be another shape (such as rounded), such as shown in FIGS. 176-183. In one or more embodiments, first surface 57 may assume the same or substantially similar profile as compared to the profile of second surface 58 (such as shown in FIGS. 152-159) such that opening 52 is a single shape. In one or more embodiments, opening 52 may include any number of notches (such as notch 88 as shown in FIGS. 136-143), indentations, or other cuts into first surface 57 and/or second surface 58 in order to better engage or correspond to the shape of various plungers 25 (such as a cruciform plunger 25) or for other reasons. As shown by the dotted lines in FIG. 144, in one or more embodiments, the thickness, and other dimensions and configurations of volume limiter's top surface 51 and bottom surface 53 may vary. As shown by the dotted lines in FIG. 144, in one or more embodiments, the width, and other dimensions and configurations of volume limiter's first bottom ledge 54 and second bottom ledge 55 may vary to fit flanges 21 of various sizes and configurations. Although not shown in FIG. 144, in one or more embodiments, the width, length, shape, and other dimensions and configurations of volume limiter's top surface 51 and bottom surface 53 may vary. Although not shown in FIG. 144, in one or more embodiments, the height, length, location, and other dimensions and configurations of volume limiter's first bottom ledge 54 and second bottom ledge 55 may vary to fit flanges 21 of various sizes and configurations.

FIG. 145 shows a bottom perspective view of the embodiment of volume limiter 50 illustrated in FIG. 144 with various components dot lined. As shown by the dotted lines in FIG. 145, in one or more embodiments, the width, circumference, depth, shape, location, and other dimensions and configurations of opening 81 and opening 82 may vary. For example, in one or more embodiments, opening 81 and opening 82 may be rectangle, square, circular, oval, oblong, or some other shape to fit flanges 21 and syringes 20 of various sizes and configurations. As shown by the dotted lines in FIG. 145, in one or more embodiments, the height, and other dimensions and configurations of retention member 80 may vary to adjust its rigidity or flexibility and correlating tension as needed. Although not shown in FIG. 145, in one or more embodiments, the width, length, shape, location, and other dimensions and configurations of retention member 80 may vary to adjust its rigidity or flexibility and correlating tension as needed. As shown by the dotted lines in FIG. 145, in one or more embodiments, the width, and other dimensions and configurations of first flange housing element 56, first flange housing arm 83 and second flange housing arm 84 may vary in order to receive flanges 21 and syringes 20 of various sizes and configurations. Although not shown in FIG. 145, in one or more embodiments, the height, shape, location, and other dimensions and configurations of first flange housing element 56, first flange housing arm 83 and second flange housing arm 84 may vary in order to receive flanges 21 and syringes 20 of various sizes and configurations.

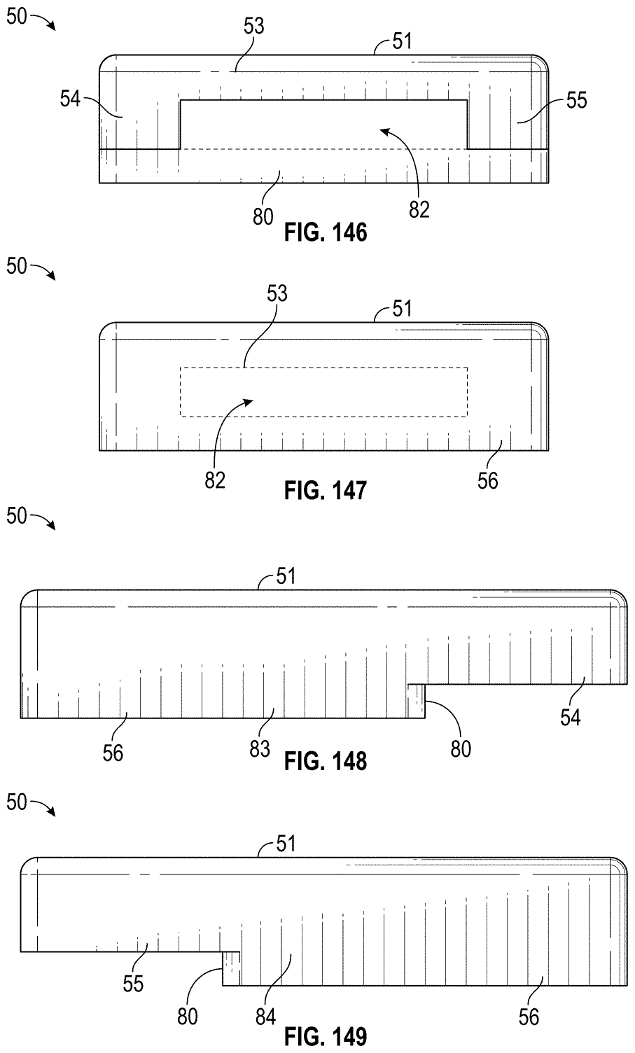

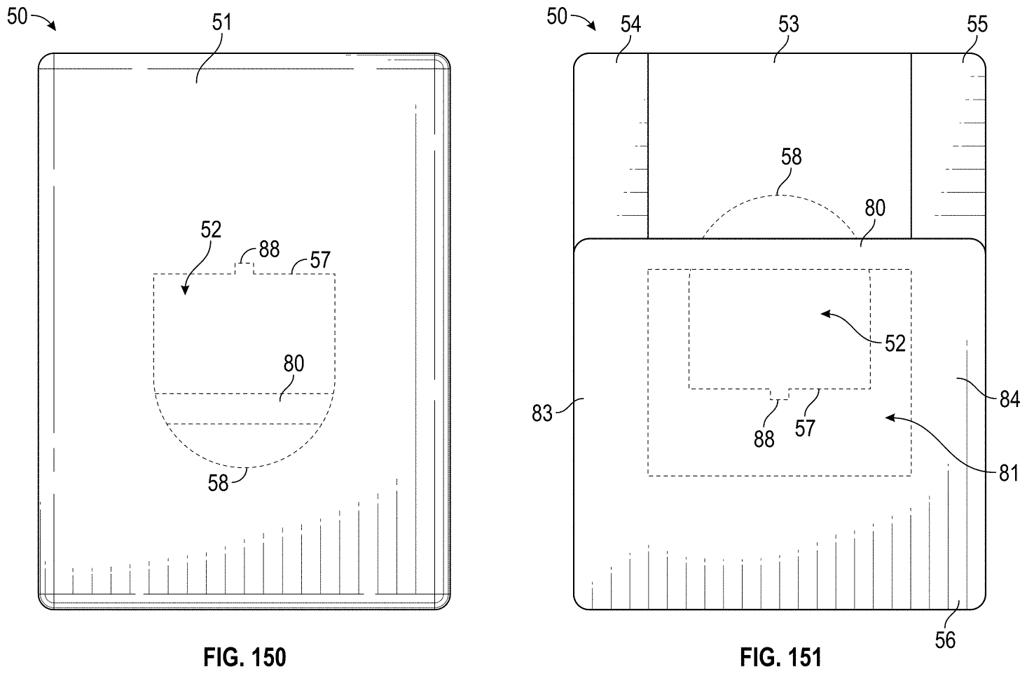

FIG. 146 shows a front view of the embodiment of volume limiter 50 illustrated in FIG. 144. FIG. 147 shows a rear view of the embodiment of volume limiter 50 illustrated in FIG. 144. As shown by the dotted lines in FIG. 147, in one or more embodiments, volume limiter 50 may not include an opening 82 that extends all the way through first flange housing element 56 (such as shown in FIGS. 10A-10E). FIG. 148 shows a right side view of the embodiment of volume limiter 50 illustrated in FIG. 144. FIG. 149 shows a left side view of the embodiment of volume limiter 50 illustrated in FIG. 144. FIG. 150 shows a top view of the embodiment of volume limiter 50 illustrated in FIG. 144. FIG. 151 shows a bottom view of the embodiment of volume limiter 50 illustrated in FIG. 144. Although not shown in FIGS. 144-151, in one or more embodiments, volume limiter 50 may be configured with various protrusions, ridges, indentations, or other configurations or materials, or combinations thereof, which may aid in securing syringe 20 to volume limiter 50. In one or more embodiments, volume limiter's 50 securement to syringe 20 may be temporary, permanent, releasable, adjustable, resilient or otherwise, or any combination thereof.

FIGS. 152-183 show various embodiments of volume limiter 50 featuring, among other things, openings 52, openings 81 and openings 82 with different shapes, sizes, diameters, height, widths, and other dimensions and configures in order to operably connect to syringes 20 and flanges 21 of different sizes and designs. For example, in one or more embodiments, volume limiter 50 may be configured to engage syringes 20 designed to hold less than 0.3 mL of medication up to and even more than 100 mL of medication. FIGS. 152-183 also show various embodiments of volume limiter 50 featuring: retention members 80, first bottom ledges 54, second bottom ledges 55, and first flange housing elements 56 (that include first flange housing arms 83 and second flange housing arms 84) with different shapes, sizes, height, widths, lengthens and other dimensions and configures in order to operably connect to syringes 20 and flanges 21 of different sizes and designs. For example, in one or more embodiments, depending on the size of the syringe 20 that a particular embodiment of volume limiter 50 is configured to engage and the amount of tension or force needed to be placed on the syringe 20, retention member 80 may be positioned such that at least a portion of it is located directly under opening 52 (such as shown in FIGS. 122-123, 134-135, 142-143, 150-151, 158-159, 166-167, 174-175) or such that it is located so that it is not directly under opening 52 (such as shown in FIGS. 182-183).

FIG. 152 shows a top perspective view of one embodiment of volume limiter 50 that is configured to be operably connected to a syringe 20 configured as a BD 3 mL syringe that includes opening 52 (fully rounded), opening 81, opening 82, retention member 80, first bottom ledge 54, second bottom ledge 55, and first flange housing element 56 (that includes first flange housing arm 83 and second flange housing arm 84), all sized and otherwise configured to operably connect to said syringe 20. FIG. 153 shows a bottom perspective view of the embodiment of volume limiter 50 illustrated in FIG. 152. FIG. 154 shows a front view of the embodiment of volume limiter 50 illustrated in FIG. 152. FIG. 155 shows a right view of the embodiment of volume limiter 50 illustrated in FIG. 152. FIG. 156 shows a rear side view of the embodiment of volume limiter 50 illustrated in FIG. 152. FIG. 157 shows a left side view of the embodiment of volume limiter 50 illustrated in FIG. 152. FIG. 158 shows a top view of the embodiment of volume limiter 50 illustrated in FIG. 152. FIG. 159 shows a bottom view of the embodiment of volume limiter 50 illustrated in FIG. 152.

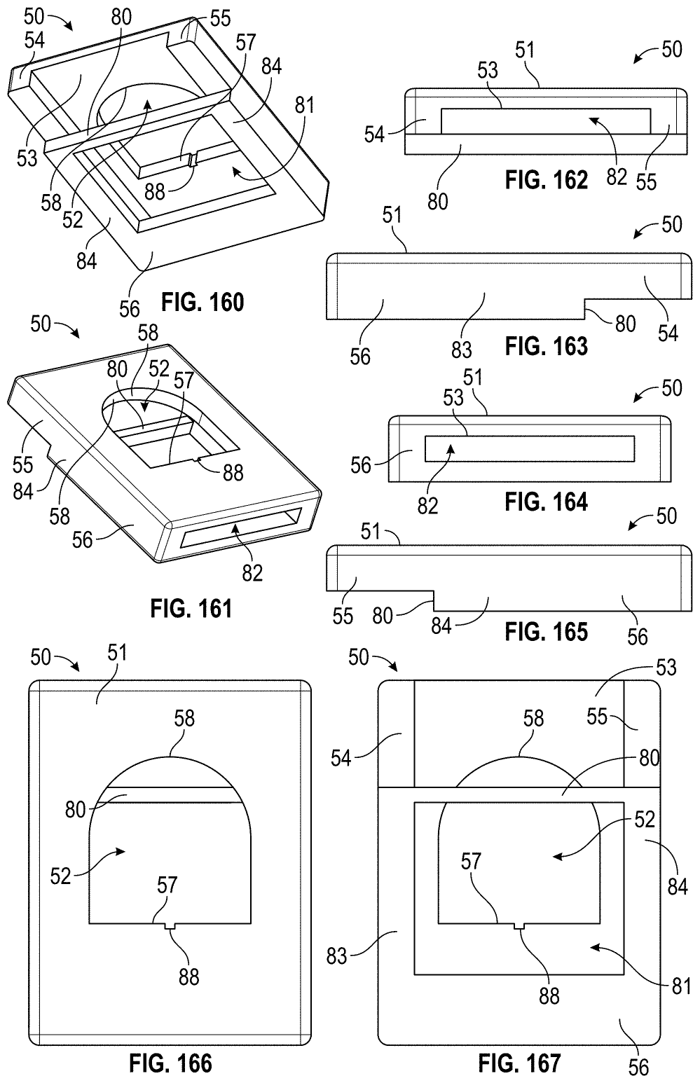

FIG. 160 shows a bottom perspective view of an alternative embodiment of volume limiter 50 configured to operably connect to a syringe 20 configured as a Braun 12 mL cruciform syringe that includes opening 52, opening 81, opening 82, retention member 80, first bottom ledge 54, second bottom ledge 55, and first flange housing element 56 (that includes first flange housing arm 83 and second flange housing arm 84), all sized and otherwise configured to operably connect to said syringe 20. FIG. 161 shows a top perspective view of the embodiment of volume limiter 50 illustrated in FIG. 160. FIG. 162 shows a front view of the embodiment of volume limiter 50 illustrated in FIG. 160. FIG. 163 shows a right view of the embodiment of volume limiter 50 illustrated in FIG. 160. FIG. 164 shows a rear side view of the embodiment of volume limiter 50 illustrated in FIG. 160. FIG. 165 shows a left side view of the embodiment of volume limiter 50 illustrated in FIG. 160. FIG. 166 shows a top view of the embodiment of volume limiter 50 illustrated in FIG. 160. FIG. 167 shows a bottom view of the embodiment of volume limiter 50 illustrated in FIG. 160.

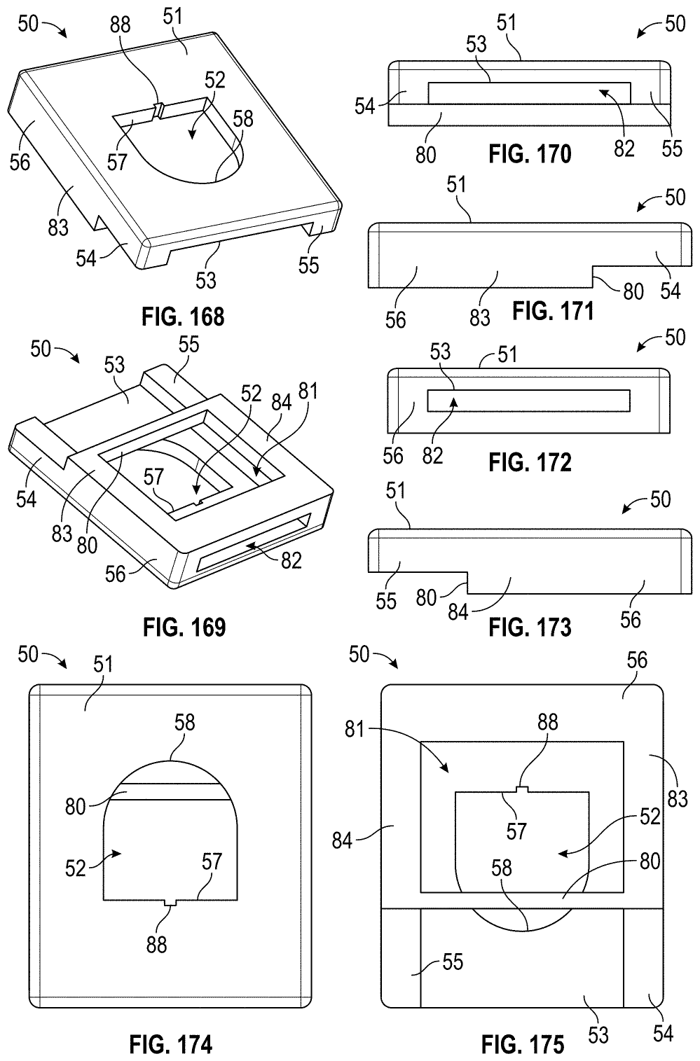

FIG. 168 shows a top perspective view of an alternative embodiment of volume limiter 50 configured to operably connect to a syringe 20 configured as a Monoject 6 mL cruciform syringe that includes opening 52, opening 81, opening 82, retention member 80, first bottom ledge 54, second bottom ledge 55, and first flange housing element 56 (that includes first flange housing arm 83 and second flange housing arm 84), all sized and otherwise configured to operably connect to said syringe 20. FIG. 169 shows a bottom perspective view of the embodiment of volume limiter 50 illustrated in FIG. 168. FIG. 170 shows a front view of the embodiment of volume limiter 50 illustrated in FIG. 168. FIG. 171 shows a right view of the embodiment of volume limiter 50 illustrated in FIG. 168. FIG. 172 shows a rear side view of the embodiment of volume limiter 50 illustrated in FIG. 168. FIG. 173 shows a left side view of the embodiment of volume limiter 50 illustrated in FIG. 168. FIG. 174 shows a top view of the embodiment of volume limiter 50 illustrated in FIG. 168. FIG. 175 shows a bottom view of the embodiment of volume limiter 50 illustrated in FIG. 168.

FIG. 176 shows a bottom perspective view of an alternative embodiment of volume limiter 50 configured to operably connect to a syringe 20 configured as an Ultimed 1 mL syringe (with squared edges) that includes opening 52, opening 81, opening 82, retention member 80, first bottom ledge 54, second bottom ledge 55, and first flange housing element 56 (that includes first flange housing arm 83 and second flange housing arm 84), all sized and otherwise configured to operably connect to said syringe 20. FIG. 177 shows a top perspective view of the embodiment of volume limiter 50 illustrated in FIG. 176. FIG. 178 shows a front view of the embodiment of volume limiter 50 illustrated in FIG. 176. FIG. 179 shows a right view of the embodiment of volume limiter 50 illustrated in FIG. 176. FIG. 180 shows a rear side view of the embodiment of volume limiter 50 illustrated in FIG. 176. FIG. 181 shows a left side view of the embodiment of volume limiter 50 illustrated in FIG. 176. FIG. 182 shows a top view of the embodiment of volume limiter 50 illustrated in FIG. 176. FIG. 183 shows a bottom view of the embodiment of volume limiter 50 illustrated in FIG. 176.

The various components and characteristics of the embodiments of volume limiter 50 shown and described in relation to FIGS. 1-38 and 116-183 may, in one or more embodiments, be mixed and matched with other each. For example, in one or more embodiments, the various configurations of openings 52, openings 81, openings 82, retention members 80, first bottom ledges 54, second bottom ledges 55, and first flange housing elements 56 (that include first flange housing arms 83 and second flange housing arms 84) may be mixed and matched with each other depending on the need. In one or more embodiments, the exact dimensions and configurations of said components may be determined by the versatility needed or not needed. The FIGS. 152-183 are not necessarily to scale in relation to each other. In one or more embodiments, volume limiter 50 may be configured to operably connect to syringes 20 made by various manufactures. In one or more embodiments, volume limiter 50 may be configured to operably connect to syringes 20 that include various devices (such as safety shields 60) made from various manufacturers. In one or more embodiments, volume limiter 50 may be configured to engage syringes 20 made of plastic, glass and/or stainless steel.

Different embodiments of the disclosure may implement the above scenario(s) and/or variations of the above scenario(s). In one or more embodiments, any of the structures, functions, and/or features of any aspect of the disclosure expressly and/or inherently described and/or illustrated herein may be combined with any of the structures, functions, and/or features of any other aspect of the disclosure expressly and/or inherently described and/or illustrated herein. In one or more embodiments, each component of the disclosure may be provided in any color.

In one or more embodiments, other modifications may be made to the embodiments illustrated in the drawings and/or otherwise disclosed herein, which may include and/or have the capacity to utilize abilities, systems, devices, articles, means, functionalities, features, methods and/or uses expressly, not expressly and/or impliedly described herein and/or illustrated in the drawings but which may be obvious to one skilled in the art, whether developed later or known at the time of filing.

It should be understood that the present systems, devices, means, methods and structures are not intended to be limited to the particular forms disclosed; rather, they are to cover all combinations, modifications, equivalents and alternatives. A system, device, article, means, method or structure that is configured in a certain way may be configured in at least that way, but may also be configured in ways that are not described or illustrated. The disclosure may be configured to function with a variety of systems, devices, means, methods, and structures. Different materials may be used for individual components. Different materials may be combined in a single component.

The present disclosure may be embodied in other specific forms without departing from its spirit or essential characteristics. It is appreciated that various features of the above described examples and embodiments may be mixed and matched to form a variety of other combinations and alternatives. It is also appreciated that devices, methods and systems disclosed herein should not be limited simply to methods, systems and devices for administering medication. The described embodiments are to be considered in all respects as illustrative and not restrictive. Other embodiments and/or implementations are within the scope of the following claims and at least all changes which come within the meaning and range of equivalency of the claims are to be embraced within their scope. The scope of the disclosure may be indicated by the appended claims rather than by any of the foregoing description.

What is claimed is:

1. A guiding element device, comprising:
a guiding element shaft;
a guiding element barrel, comprising: a first recessed portion, a second recessed portion, a third recessed portion, a fourth recessed portion, a fifth recessed portion, and a sixth recessed portion;
a transition section, comprising a top surface and a bottom surface;
a first support arm, a second support arm, and a third support arm, wherein each support arm is operably connected to the transition section and the guiding element barrel and is capable of articulating; and
a first window, a second window, and a third window, wherein each window is located between the transition section and the guiding element barrel.

2. The guiding element device of claim 1, wherein the transition section further comprises: a first slit, a second slit, a third slit, a fourth slit, a fifth slit, and a sixth slit.

3. The guiding element device of claim 2,
further comprises:
a fourth support arm operably connected to the transition section and the guiding element barrel and is capable of articulating;
a fourth window located between the transition section and the guiding element barrel; and
a seventh slit and an eighth slit;
wherein the guiding element barrel further comprises a seventh recessed portion and an eighth recessed portion.

4. The guiding element device of claim 3, wherein each of the recessed portions comprises an angled edge that slopes towards the bottom surface of the transition section.

5. The guiding element device of claim 4, wherein the slope of each angled edge is at least 45 degrees relative to a longitudinal axis of the guiding element barrel.

6. The guiding element device of claim 3, wherein:
the first recessed portion and the second recessed portion meet to form a first channel;
the third recessed portion and the fourth recessed portion meet to form a second channel;
the fifth recessed portion and the sixth recessed portion meet to form a third channel; and
the seventh recessed portion and the eighth recessed portion meet to form a fourth channel.

7. The guiding element device of claim 6, wherein each of the channels is rounded and is located on an exterior surface of the guiding element barrel.

8. A volume limiter device, comprising:
a top surface and a first opening on the top surface through which a plunger from a syringe is insertable;
a bottom surface;
a first bottom ledge and a second bottom ledge located on the bottom surface;
a first flange housing element for housing a flange of a syringe, wherein the first flange housing element is located on the bottom surface; and
and a retention member capable of resiliently engaging a syringe.

9. The volume limiter device of claim 8, wherein the first opening comprises a first surface and a second surface.

10. The volume limiter device of claim 9, wherein the first surface assumes a first contour and the second surface assumes a second contour which is alternative to the first contour.

11. The volume limiter device of claim 10, wherein the first surface of the first opening further comprises a notch capable of engaging a cruciform plunger.

12. The volume limiter device of claim 8, wherein the first flange housing element comprises a first flange housing arm and a second flange housing arm.

13. The volume limiter device of claim 12, wherein the first flange housing arm is integral to the first bottom ledge and the second flange housing arm is integral to the second bottom ledge.

14. The volume limiter device of claim 12, wherein the retention member is located between the first flange housing arm and the second flange housing arm.

15. The volume limiter device of claim 12, wherein the first flange housing element further comprises a second opening through which the flange of the syringe is insertable and which second opening is located under the bottom surface of the volume limiter and is in between the first flange housing arm and the second flange housing arm.

16. The volume limiter device of claim 12, wherein the first flange housing element further comprises a third opening through which the flange of the syringe is insertable and which third opening is located at the rear of the volume limiter.

17. A medication delivery device, comprising:
a syringe, comprising:
a flange;
a syringe barrel for housing medication; and
a plunger comprising an engagement section;
a needle;
a guiding element, comprising: a guiding element shaft capable of housing the needle and a guiding element barrel capable of engaging a vial; and
a volume limiter, comprising:
a top surface;
a first opening on the top surface through which the plunger is insertable; and
a retention member capable of resiliently engaging the syringe.

18. The medication delivery device of claim 17, wherein the guiding element further comprises:
a transition section, comprising a top surface and a bottom surface;
a first support arm, a second support arm, and a third support arm, wherein each support arm is operably connected to the transition section and the guiding element barrel and is capable of articulating; and
a first window, a second window, and a third window, wherein each window is located between the transition section and the guiding element barrel.

19. The medication delivery device of claim 18, wherein the guiding element barrel further comprises: a first recessed portion, a second recessed portion, a third recessed portion, a fourth recessed portion, a fifth recessed portion, and a sixth recessed portion.

20. The medication delivery device of claim 17, wherein the volume limiter further comprises:
a bottom surface;
a first bottom ledge and a second bottom ledge located on the bottom surface; and
a first flange housing element for housing the flange of the syringe, wherein the first flange housing element is located on the bottom surface.
